(19) United States
(12) Patent Application Publication
(10) Pub. No.: US 2005/0191385 A1
(43) Pub. Date: Sep. 1, 2005

(54) Natural product derivatives as food supplements and pharmaceuticals

(76) Inventor: Daniel K. Amato, Wellington, FL (US)

Correspondence Address: John P. Luther, Esq.;Newman & Newman, LLP, Suite 610, 505 Fifth Avenue South, Seattle, WA, 98104, US

(21) Appl. No.: 10/973,778

(22) Filed: Oct. 25, 2004

(51) Int. Cl. A61K 38/17; A61K 31/56; A01N 37/18; A61K 38/00; A01N 45/00; A21D 2/24; A21D 8/02

(52) U.S. Cl. 426/23; 514/2; 514/26

(57) ABSTRACT

Then present invention provides new and useful derivatives of phyto-derived tetracyclic compounds possessing steroidal-like activity, and in some instances both amino acid/oligo-peptide and steroidal-like activity, uses thereof and methods of production.

### FIELD OF THE INVENTION

The present invention relates to natural occurring and natural product-derived food products and drugs, and more specifically to derivatives of ecdysteroids and other tetracyclic hydroxylated and polyhydroxylated natural occurring and natural product-derived compounds which are useful as food supplements and drugs, and to nutritional, pharmaceutical and dietetic preparations thereof and the use of such products for enhanced body preformance in humans and animals.

### BACKGROUND OF THE INVENTION

Copyright notice 2004 Daniel Amato. All rights reserved. A portion of the disclosure of this patent application/patent document contains material which is subject to copyright protection. The copyright owner has no objection to the facsimile reproduction by anyone of the patent document or the patent disclosure as it appears in the United States Patent and Trademark Office file or records.

Many compounds originally isolated from plant sources have been used as drugs and/or food supplements or otherwise growth promoters for thousands of years. For example, hydroxylated and polyhydroxylated steroid compounds found in nature and various analogs thereof, such as phytoecdysteroids, have been found to stimulate protein synthesis and/or otherwise have found use as tissue growth promoters, with some examples including cyasterone, turkesterone, viticosterone E and 2-acetate hyroxyecdysone. In particular, for example, ecdysteroid stimulation of protein synthesis (and, perhaps, reduction of protein catabolism) has been demonstrated in pigs to increase lean body mass with improved nitrogen retention and body weight increases, with concomitantly decreased food consumption. Kratky et al., Effect of 20-hydroxyecdysone on the protein synthesis of pigs., Zivocisna Vyroba 42: 445-451 (1997). Additional studies employing diets supplemented with ecdysteroid-containing plants, such as Rhaponticum carthamoides, have reported similar growth-promoting effects on pigs over a 30-day period. Selepcova et al., Use of Rhaponticum carthamoides Wild., In animals nutrition., Cultivation, Harvesting and Processing of Herbs, Meeting in The High Tatras, Slovak Republic, June 15-16, Book of Abstracts, P. 76. Ecdysteroids have also been shown to produce similar effects as anabolic steroids with increased physical performance without training and to increase muscle ATP content in vitamin D-deprived rats. Chermnykh et al., Effects of methandrostenolone and ecdysterone on physical endurance of animals and protein metabolism in the skeletal muscles., Farmakologiya I Toksikologiya 6: 57-62 (1988) and Kholodova et al., Effects of vitamin D3 and 20-hydroxyecdysone on the content of ATP, creatine phosphate, carnosine and Ca+ in skeletal muscles., Ukrainskii Biokhimicheskii Zhurnal 69: 3-9 (1997). Further, phytoecdsteroids have shown use in wound-healing by shorting the duration of skin repair and to possess psoriasis-inhibiting effects, with many reported uses in the cosmetics field. Detmar et al., Effects of ecdysterone on the differentiation on of normal human keratinocytes in vitro. European Journal of Dermatology 4: 558-562 (1994) and, for example, Lin N et al., Beta-Ecdysone containing skin-protecting cosmetics. Chemical Abstracts 111: 239323e (1989). Other reported uses of ecdysteroids have been to stimulate hepatic functions, to improve renal, heart and lung functions, to improve immune systems by increasing antibody-forming cells, provide antoxidant activity, and to display antimicrobial activity. Lafont et al., Practical uses for ecdysteroids in mammals including humans: an update, Journal of Insect Science, 3: 7 (2003).

The biological activity of steroid compounds, such as phtohydroxylated and ployhydroxylated steroid compounds, incusive of 20-hydroxyecdysone, has generally been attributed to a cis-fused A/B-ring structure with binding activity of compounds to receptors taking place over the A- and B-rings on the alpha or beta side, as planer trans-fused epimers are for the most part devoid of activity. Analogs with differing A-ring polarity have been thought to effect activity, such as to impart activity, by increasing attraction to receptor active sites. See generally, Hom et al., Insect Moulting Hormones. The Synthesis and Biological Activity of Some Alkylated Ecdysone Analogs, Aust. J. Chem., 35: 629-40 (1982). In other modes of activity it is thought that lipophilically enhanced analogs will be adsorbed/absorbed at a faster rate via lymphatic circulation, and to potentially bypass liver degradation for increased bioavailability to the bloodstream. Some examples of this nature include testosterone derivatives with 17 alpha alkylation on D-ring portions with methyl and ethyl groups. Other examples have described increased lymphatic absorption through derivative formation with long chain alkyl ester groups, such as testosterone undecanoate, and enyl ether groups. Oral activity is thought to be enhanced by such side chain derivatization being easily hydrolized after being lymphatically adsorbed.

Anabolic steroids are generally provided as synthetic derivatives of the naturally occurring steroid hormone testosterone. Some naturally occurring testosterone derivitives, such as 11-ketotestosterone, a naturally occurring fish steroid, have found important uses, such as displaying anabolic steroid-like activity, and more recently modulation of human glucocorticoids. Modulation of glucocorticoid activity is important in regulating physiological process in a wide array of human tissues and organs, as glucocorticoids play an important role in the regulation of vascular tone and blood pressure. As is well known, glucocorticoids may bind to and activate receptors to potentiate vasoconstrictive effects of catecholamines and angiotensin. Compounds such as 11-ketotestosterone have been reported to increase the concentration of glucocorticoids in tissues of a subject, and to be effective as 11, B-HSD1 reductase inhibitors in the treatment of lung disorders, such as pulmonary hypertension in humans, and as 11 p-HSD1 reductase inhibitors in the treatment of hypertension, including arterial hypertension and pulmonary hypertension, and other blood pressure disorders in humans, such as high blood pressure, cardiomyopathy, congestive heart failure, chronic heart failure, left ventricular hypertrophy, myocardial infarction and acute heart failure. Such glucocorticoid modulation may also affect insulin insensitivity and various neurological disorders. See, for example, International Patent Application No. PCT/US02/41356 03059267.

All steroids, including naturally occurring and phyto-derived steroids, are for the most part water insoluble and, as is known, are detectable in a human's system for a long while. Because steroids are chemically “fatty” and lipophillic, as opposed to being water soluble and hydrophilic, they may readily diffuse into lipids, or body fat, and through lipid bi-layers of cell walls and plasma membranes, thereafter passing relatively easily into cells. Once into a cell's cytoplasm, steroids may then bind to intracellular protein receptors forming a steroid-receptor complex which can bind to specific DNA sequences, referred to as hormone response elements, and which may effect the activation and/or inhibition of the transcription of adjacent genes. This effect on gene expression may be far reaching as the products of genes oftentimes turn directly on the nature of the steroid-receptor complex (hereinafter “primary response genes”), and in which such gene products are themselves regulatory proteins which activate or suppress other genes in turn to produce a delayed or secondary response. Which genes are turned on or off depends on cell type, with many different cells responding differently to steroid hormones, such as muscle cells responding to testosterone by exhibiting cell growth and breast cells showing inhibition in milk gland development. As steroids are bound to carriers they are not susceptible to osmotic loss via the kidneys and other disposal mechanisms for water soluble compounds and may persist in the blood or tissue fluids for days or longer creating a long lasting response, and perhaps time enough for adverse or unwanted side effects to manifest themselves.

Recently, however, there has been some interest in producing more water soluble steroids in an effort to minimize various reported adverse effects of previously long lasting steroids while reaping such desired effects as energy boosting, cell repair and facilitation of muscle build and recovery after an injury, and the promotion of protein growth/muscle growth in general. Some water soluble derivatives of steroids have been reported which are removed from detectable levels in a person in a relatively short time, such as within 24-72 hours. In addition to the avoidance of adverse side effects, many users, such as athletes, find such water soluble derivatives desirable as the detection in one's system of such compounds is short lived, especially when used with diuretics which may mask their use when discreetness of use is desired.

In view of the many beneficial uses and attributes of phyto- and other naturally derived and occurring food supplements and drugs, it would be an asset to provide new and useful tetracyclic derivatives which possess nutritional and/or therapeutic bioactivity, including both lipophilic and hydrophilic derivatives, and methods of production of such compounds, and uses therefore to increase the arsenal of beneficial new food supplements and pharmaceuticals for human and animal consumption.

### SUMMARY OF THE INVENTION

The present invention provides several new and useful derivatives of naturally occurring and/or phyto-derived tetracyclic compounds possessing steroid-like activity and uses therefore, and methods of production.

The inventive compounds and methods of production and use thereof are more fully explained and will be more fully understood with reference to the following Detailed Discussion of Preferred Embodiments.

### DETAILED DISCUSSION OF PREFERRED EMBODIMENTS

All patent references, published patent applications and literature references referred to or cited herein are expressly incorporated by reference to the same extent as if each were specifically and individually indicated to be incorporated by reference. Any inconsistency between these publications and the present disclosure is intended to and shall be resolved in favor of the present disclosure.

Throughout this specification and the claims which follow, unless the context requires otherwise, the word “comprise” and variations such as “comprises” and “comprising” will be understood to imply the inclusion of a stated compound, bio-active agent, carrier, vehicle, adjuvant or excipient or other material or group thereof, but not the exclusion of any other compound, bio-active agent, carrier, vehicle, adjuvant, or excipient or other material or group thereof.

The language “in combination with” another agent includes co-administration of the compound or compounds of the invention (“compound(s)”) and the agent, administration of the compound(s) of the invention first, followed by the other agent and administration of the other agent first, followed by the compound(s) of the invention.

The present invention provides several novel esterified derivatives of naturally occurring and/or phyto-derived tetracyclic compounds which possess steroid-like activity, and in some embodiments amino acid/oligo-peptide activity as well, and novel formulations of such compounds with pharmaceutically acceptable carriers. All of the inventive compounds are useful as nutrients, food supplements and pharmaceuticals for mammals. Such compounds are of a formula selected from the group consisting of: 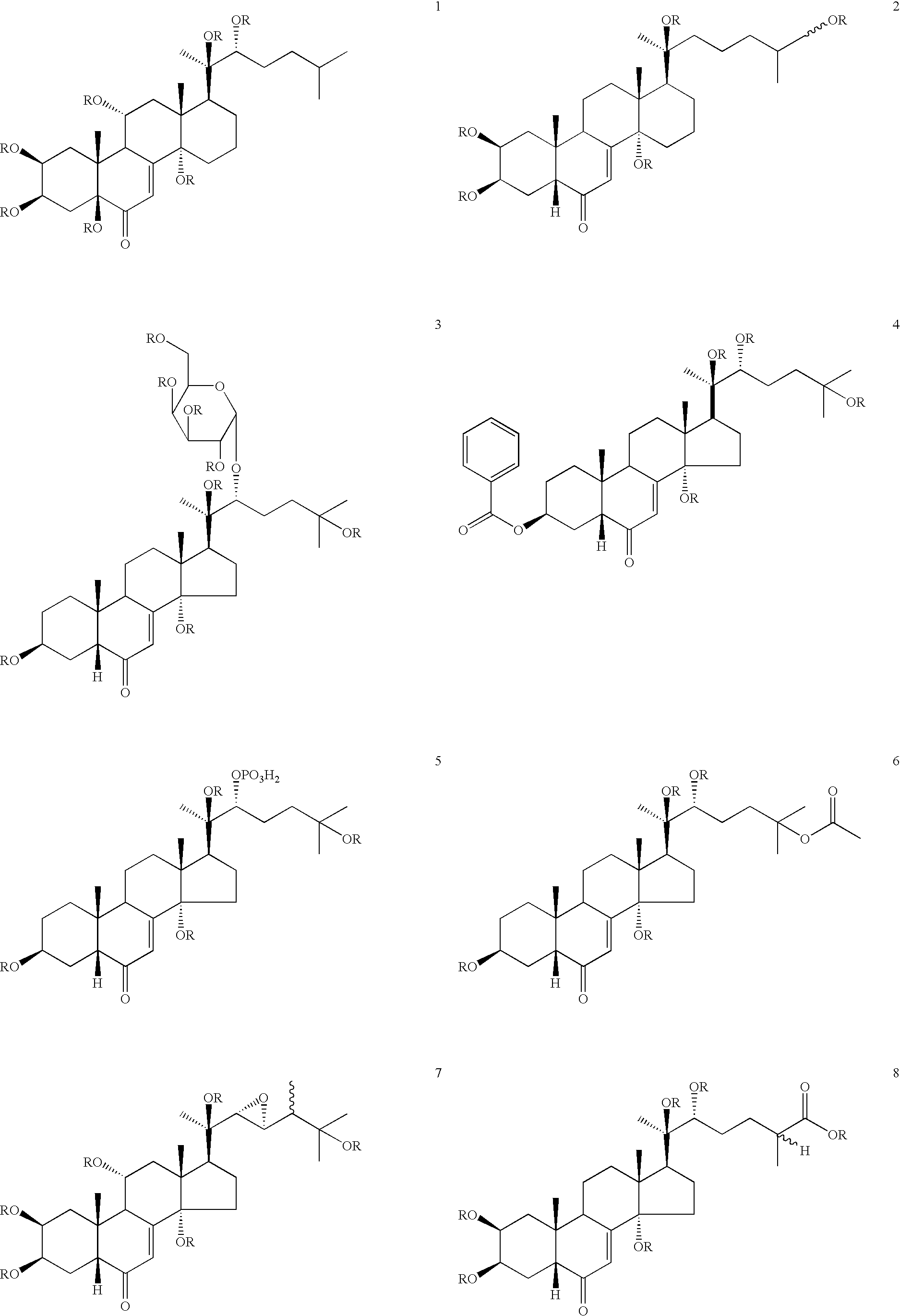 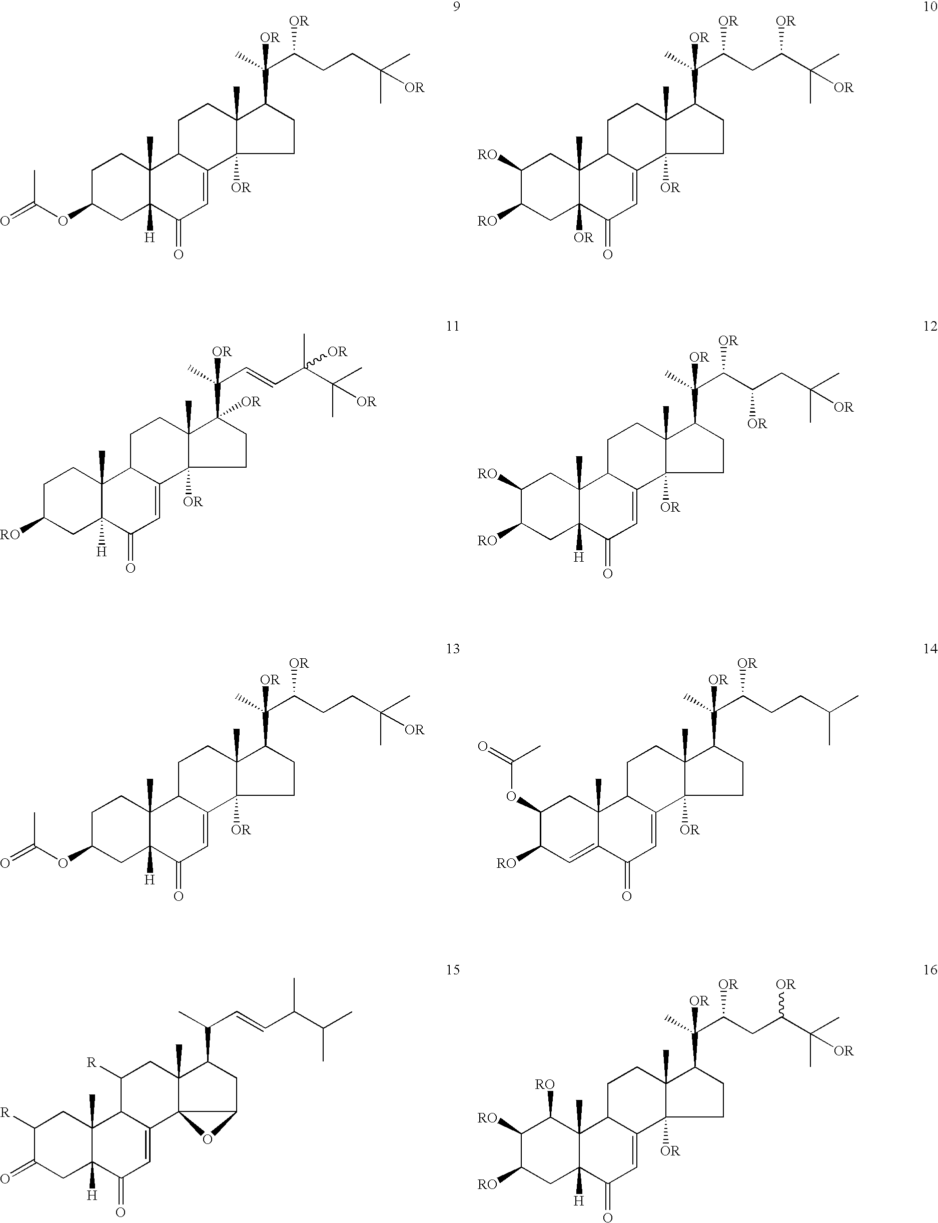 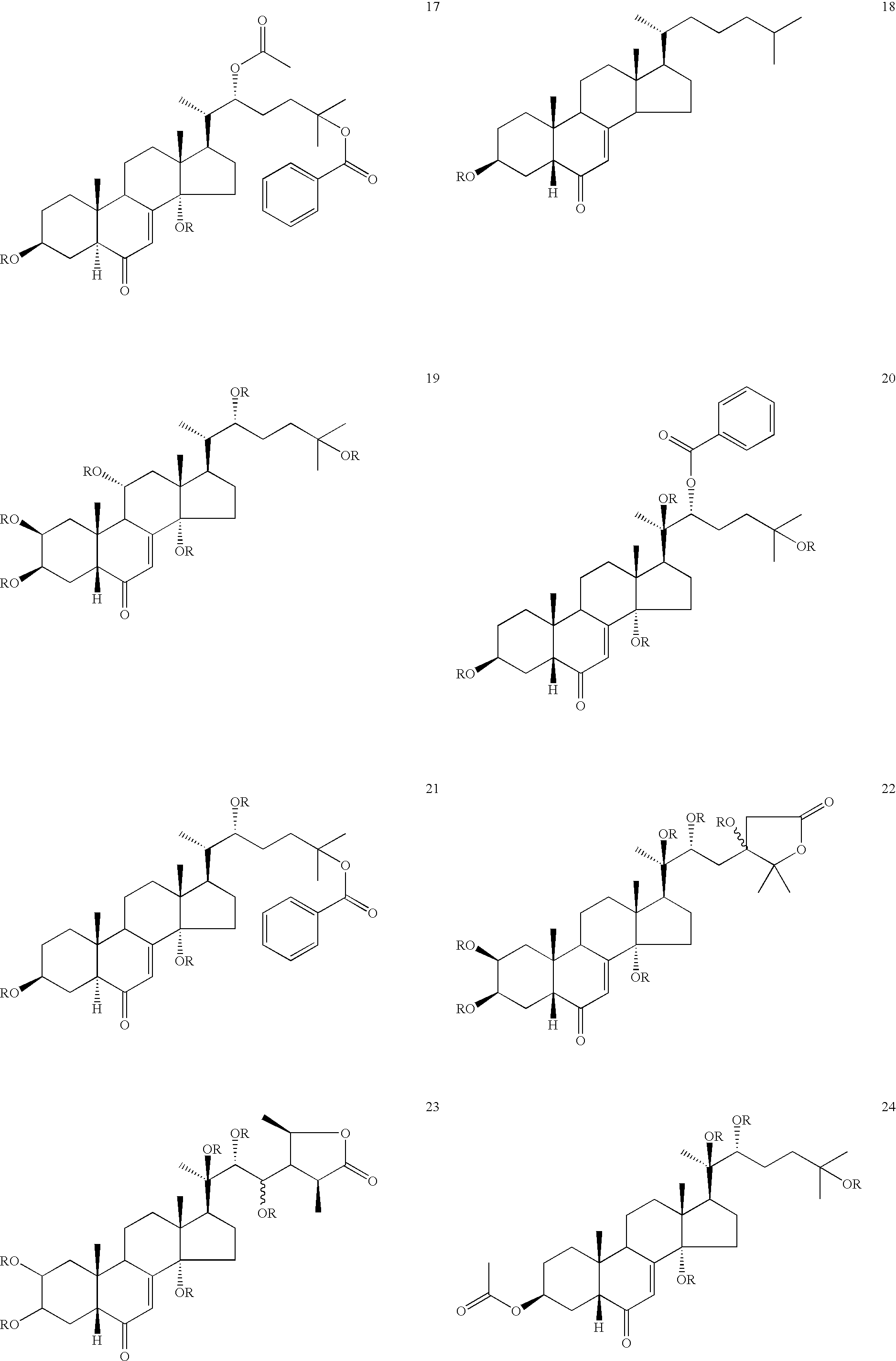 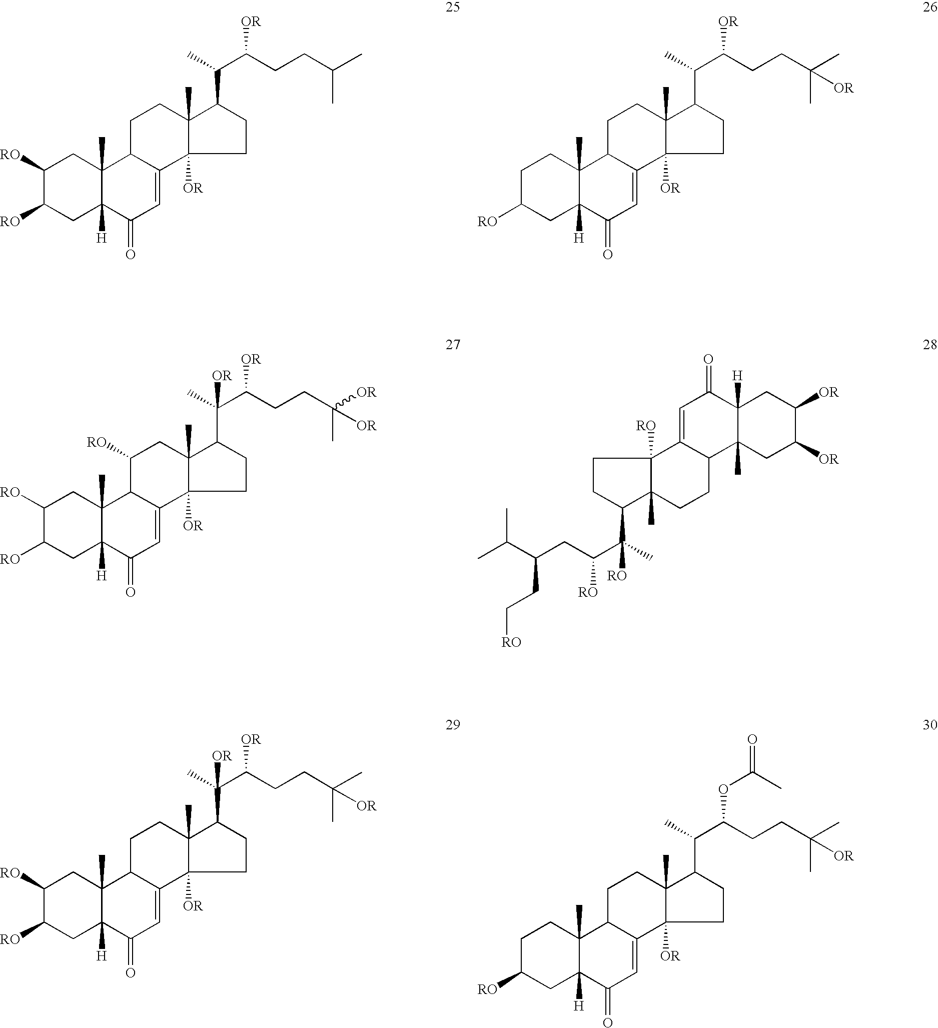 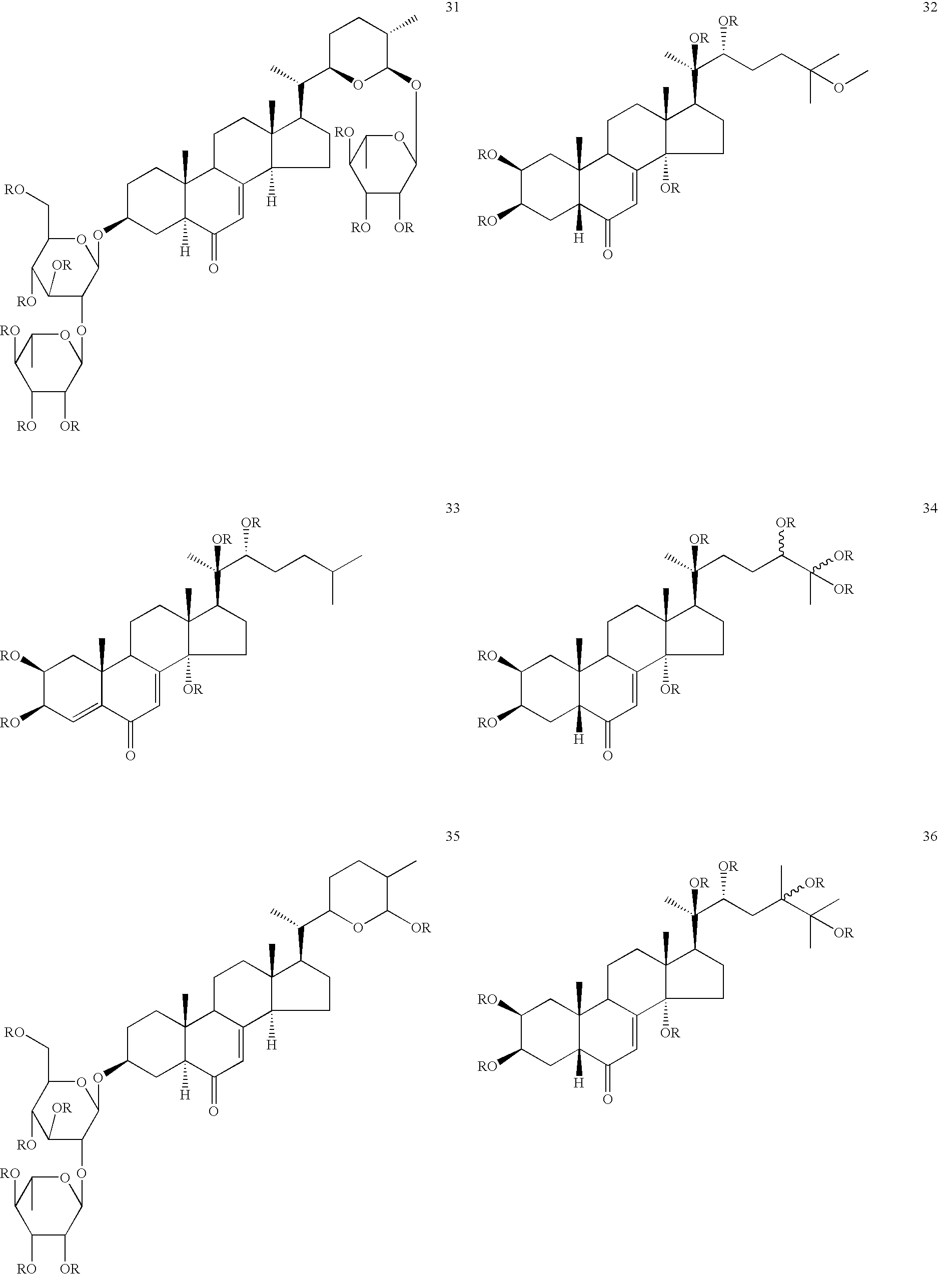 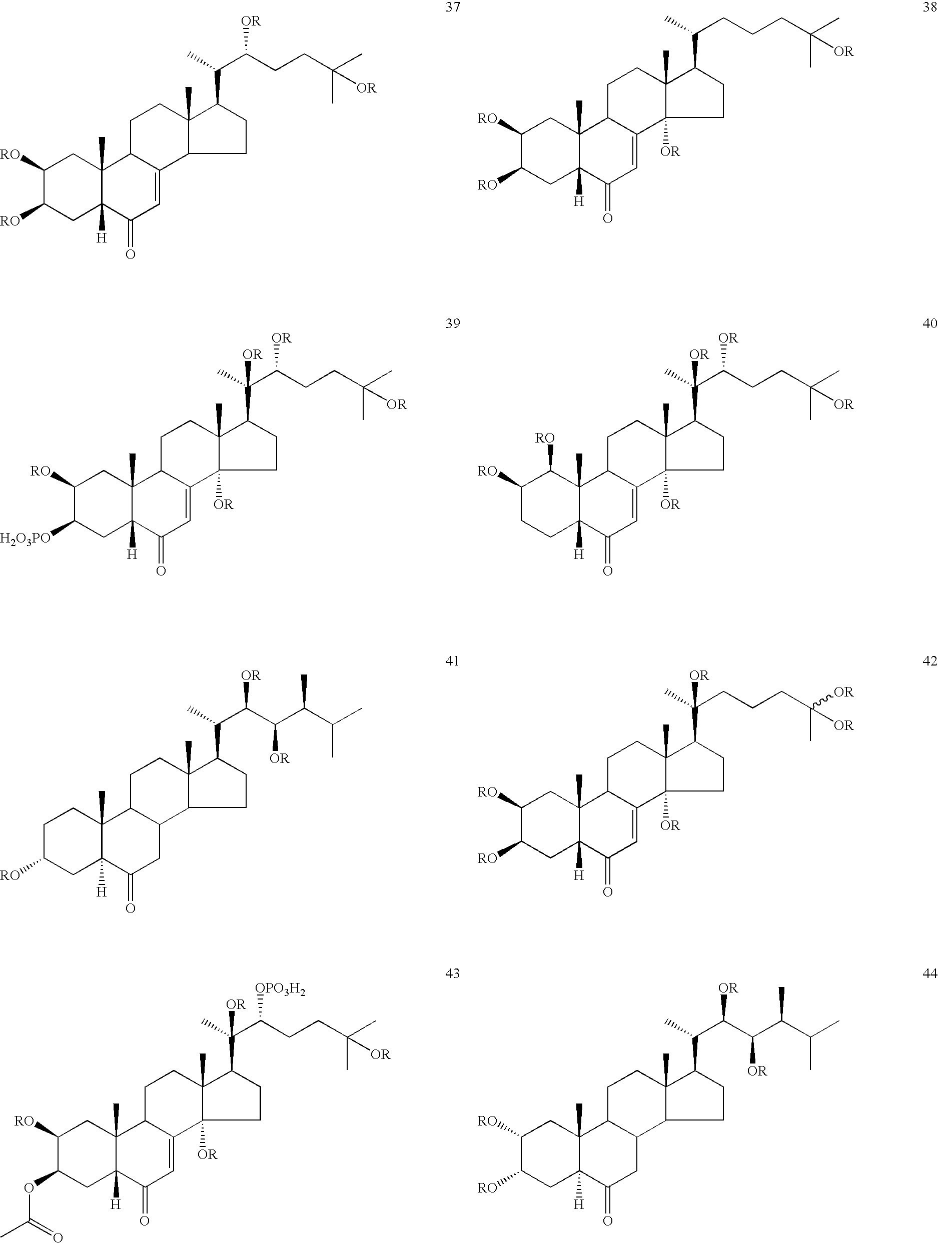 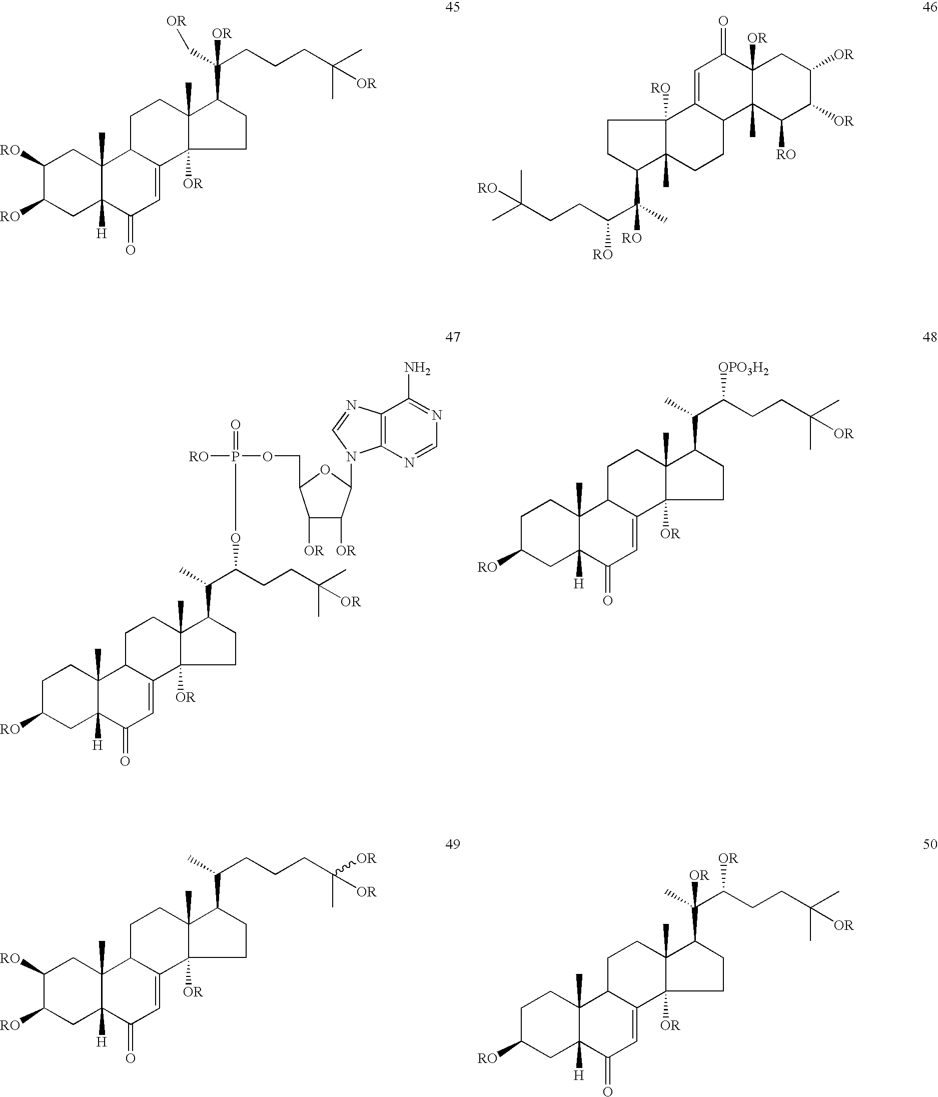 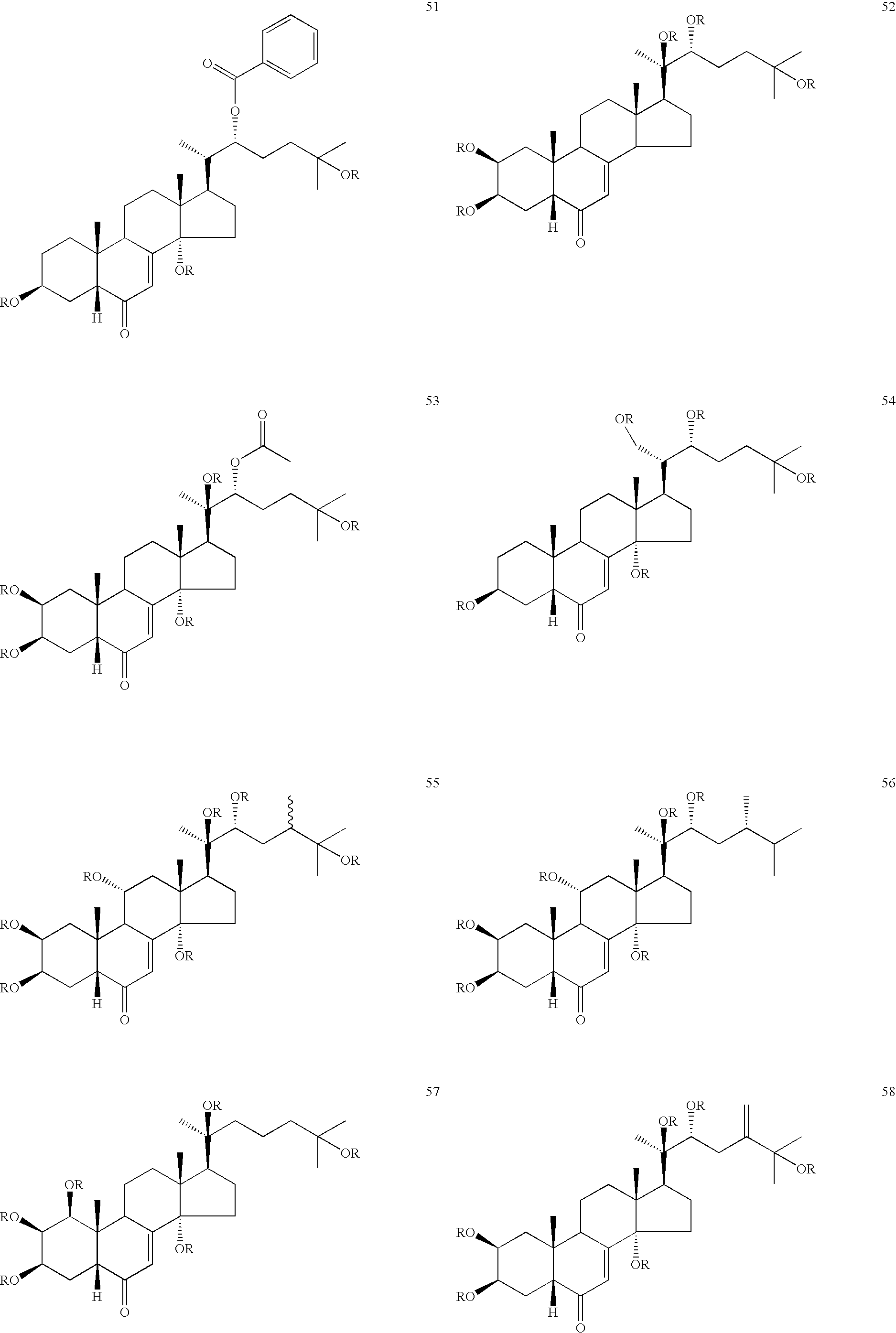 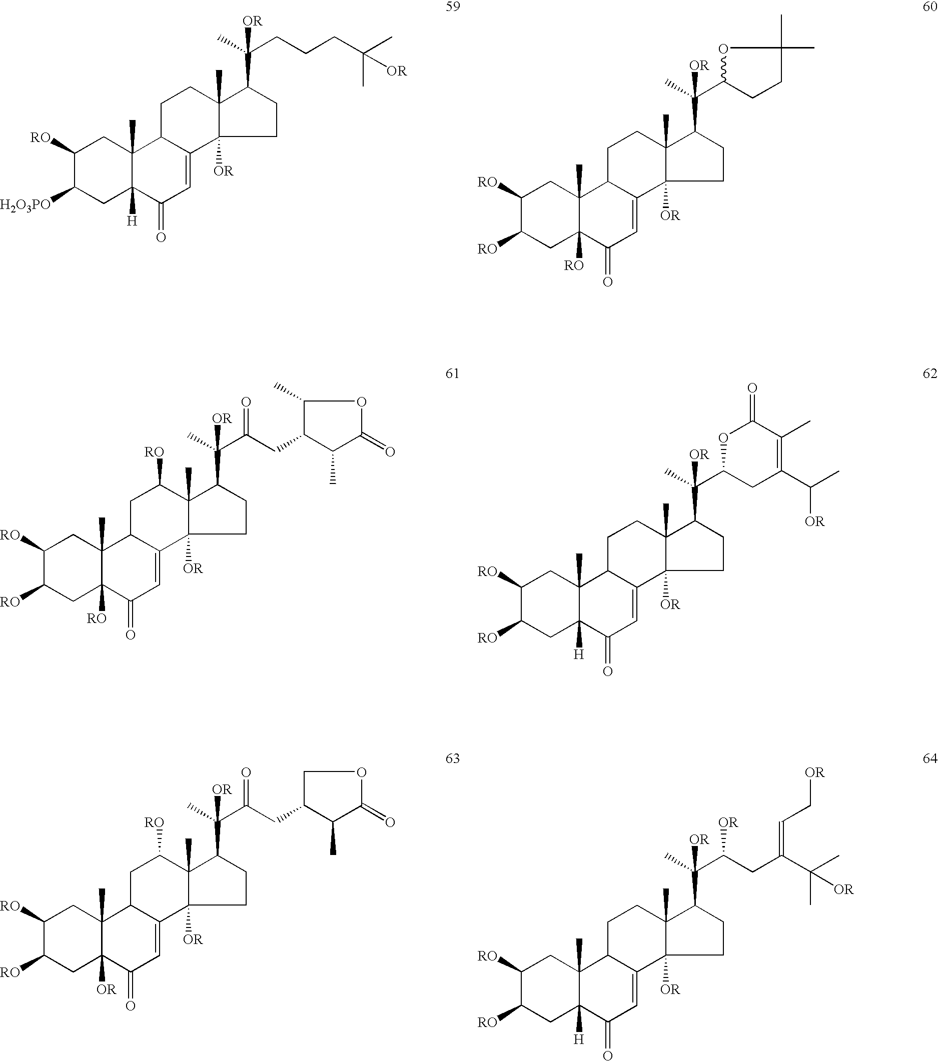 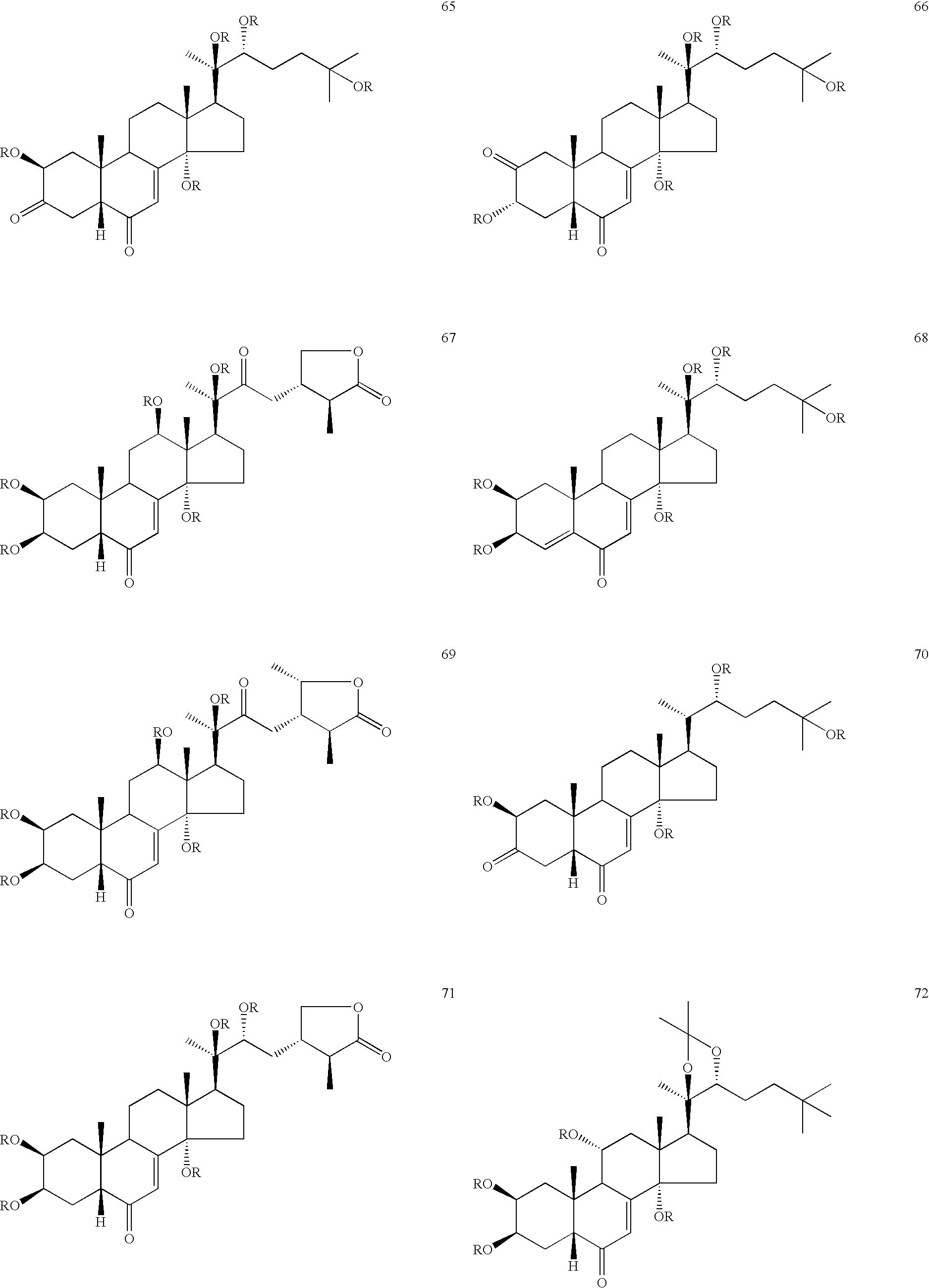 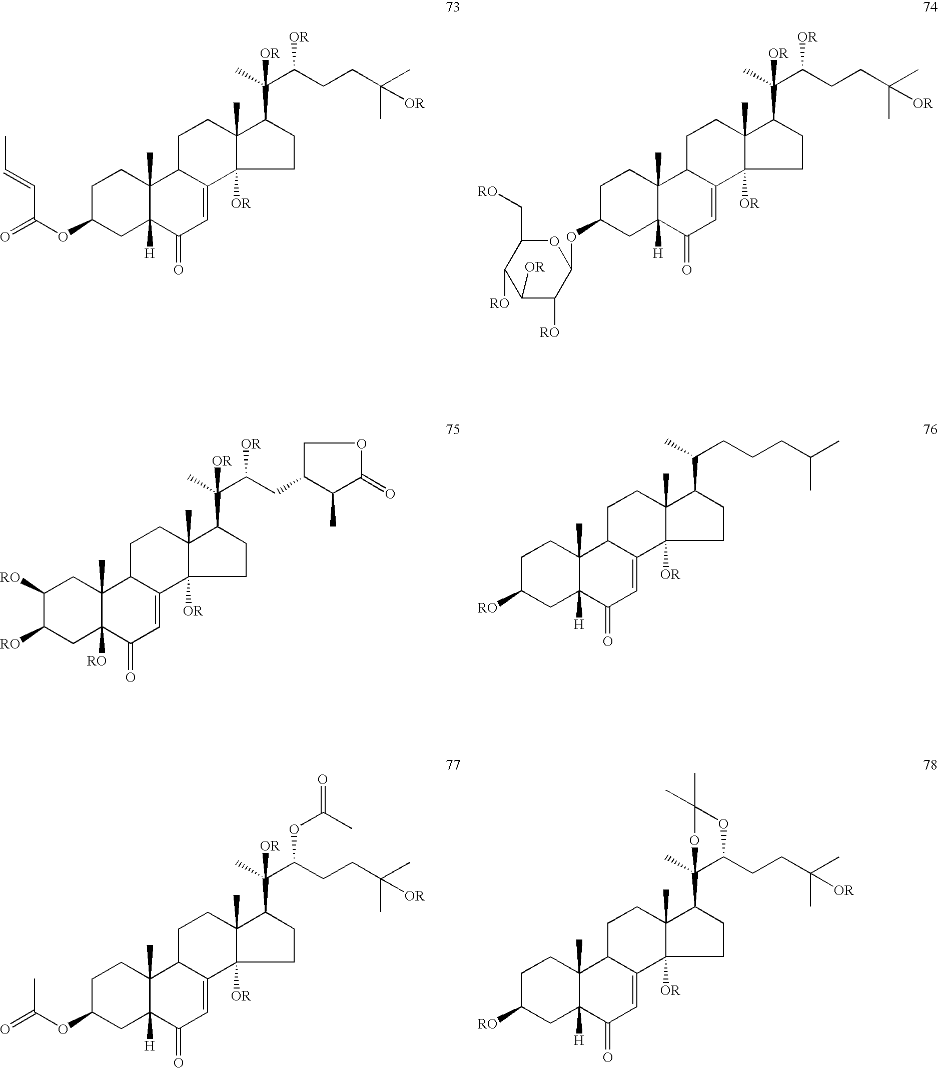 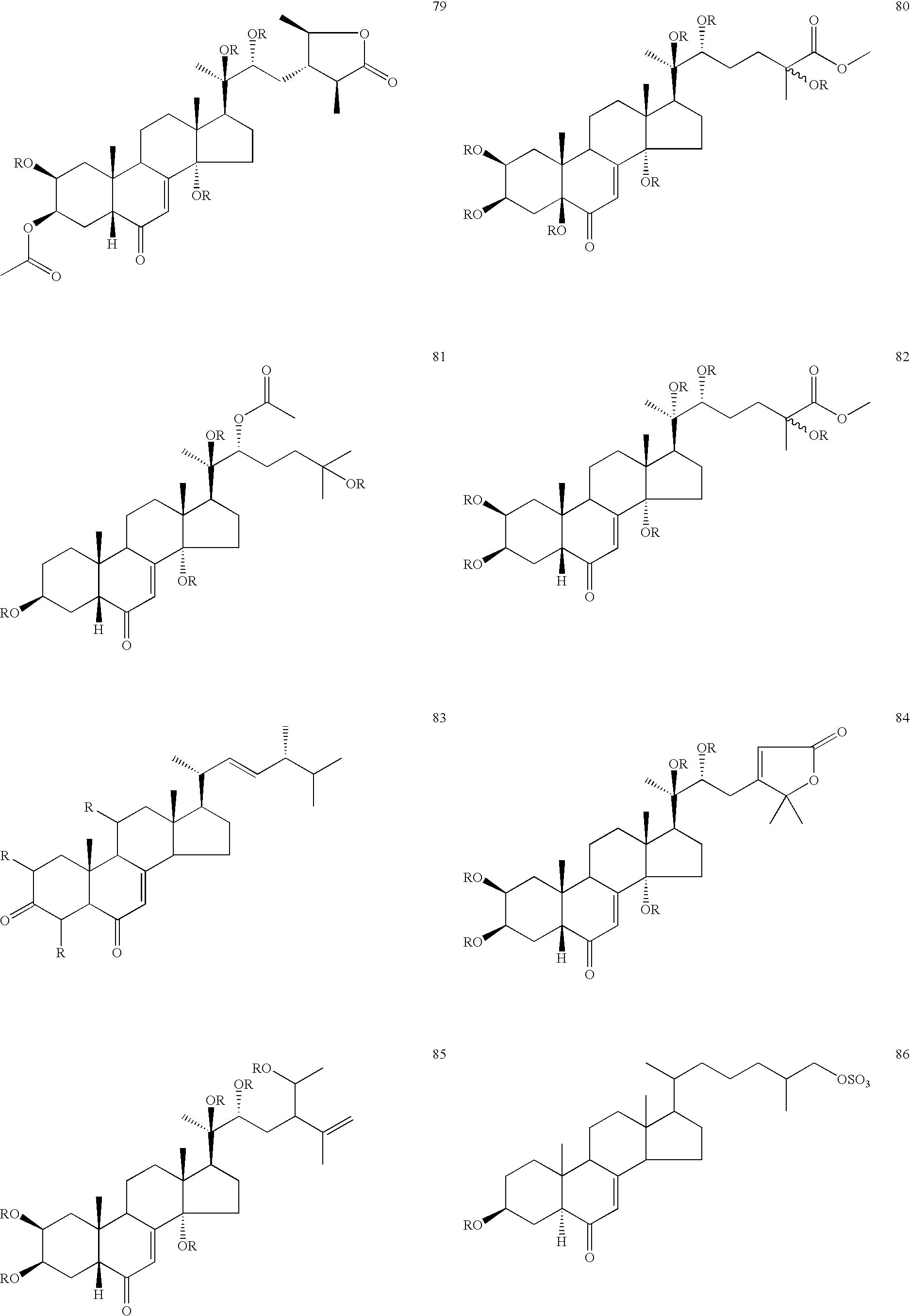 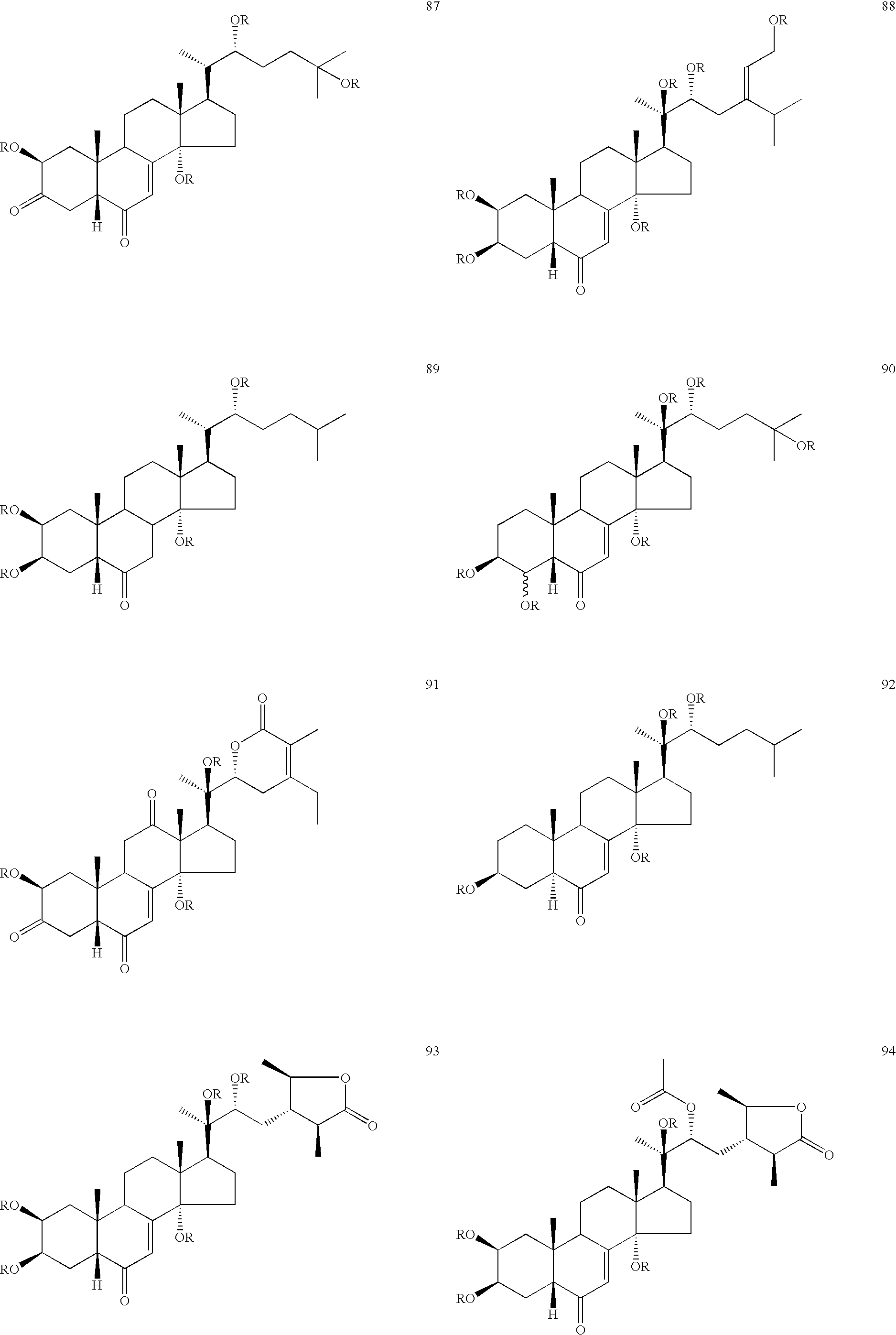 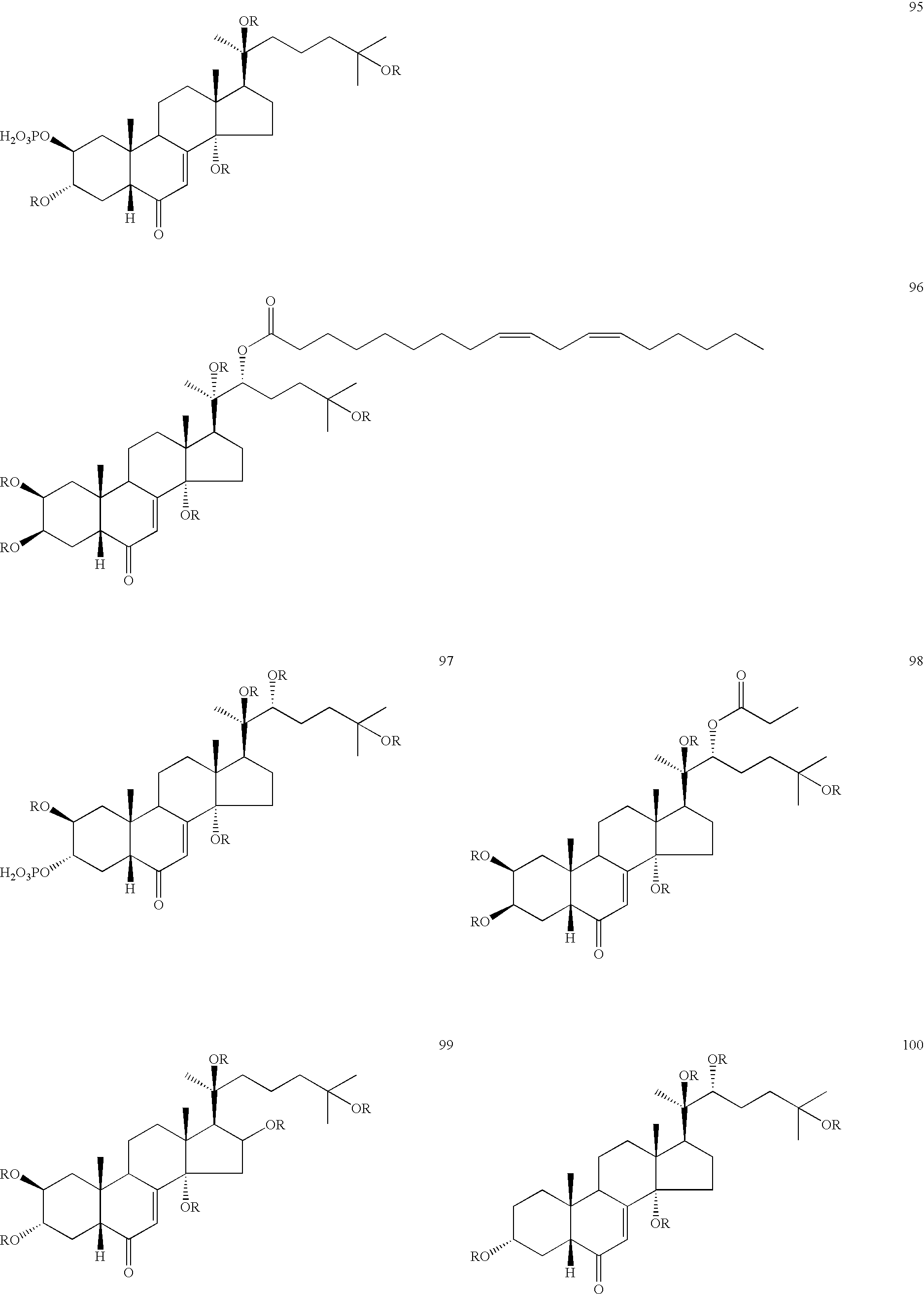 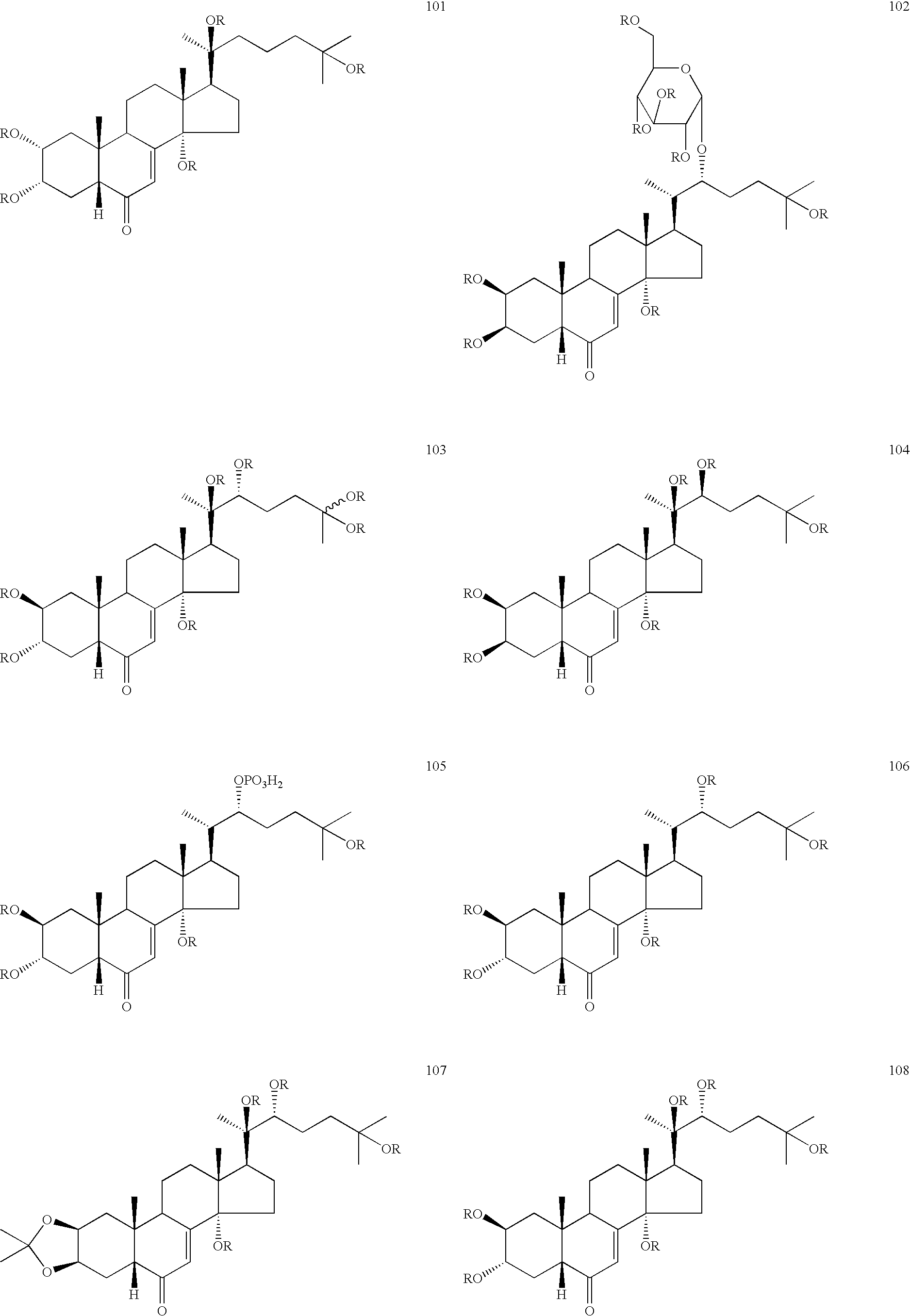 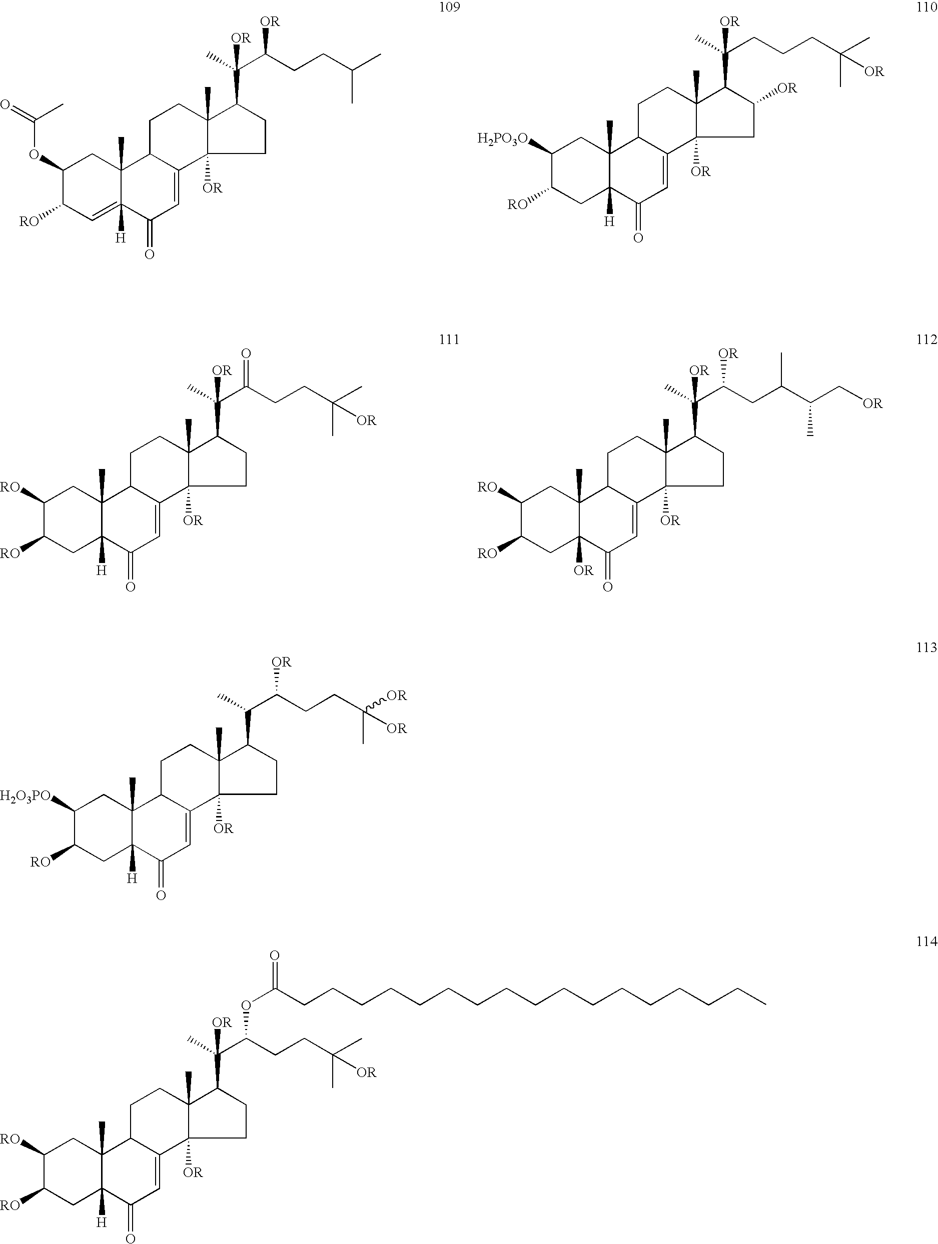 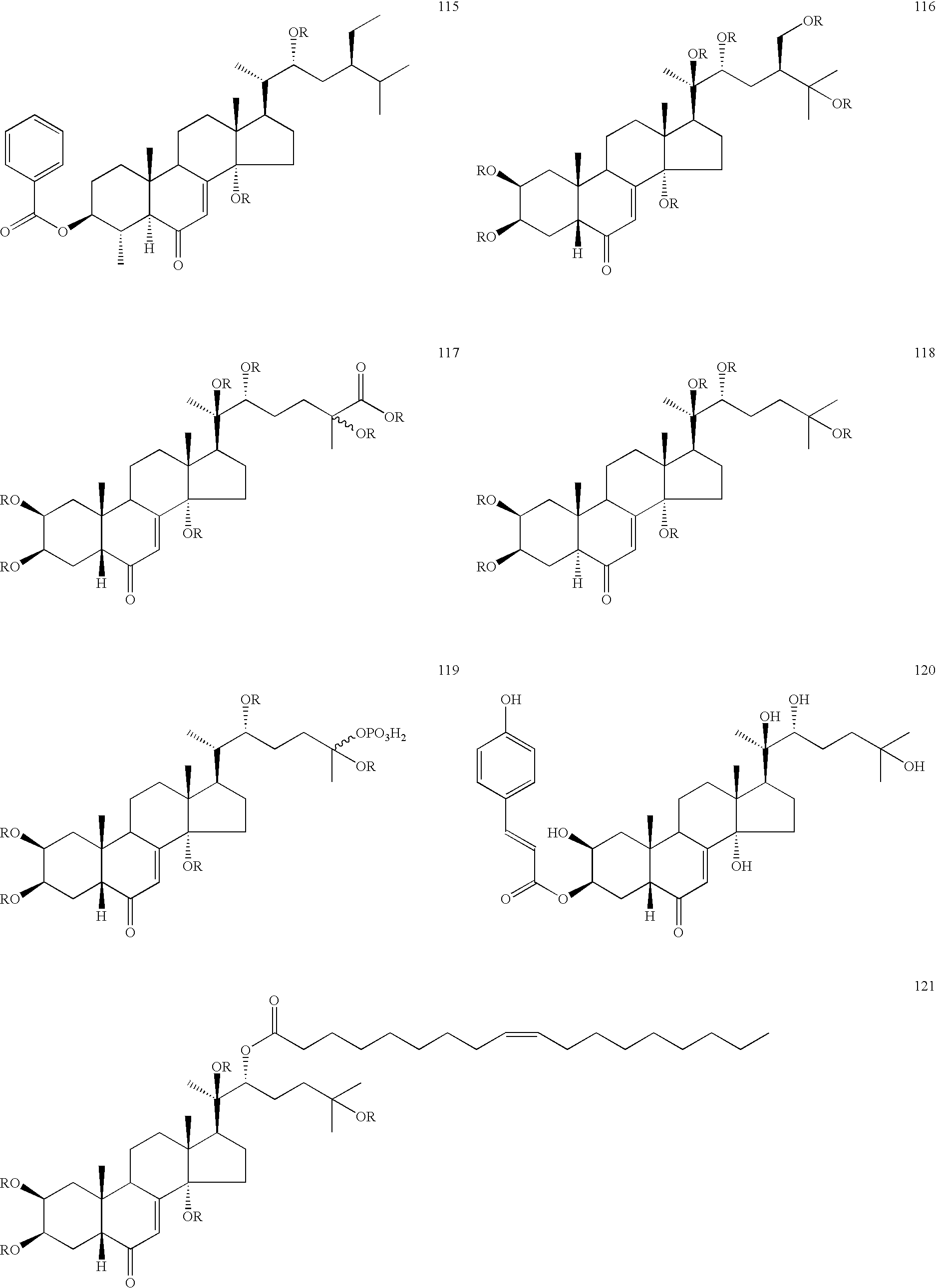 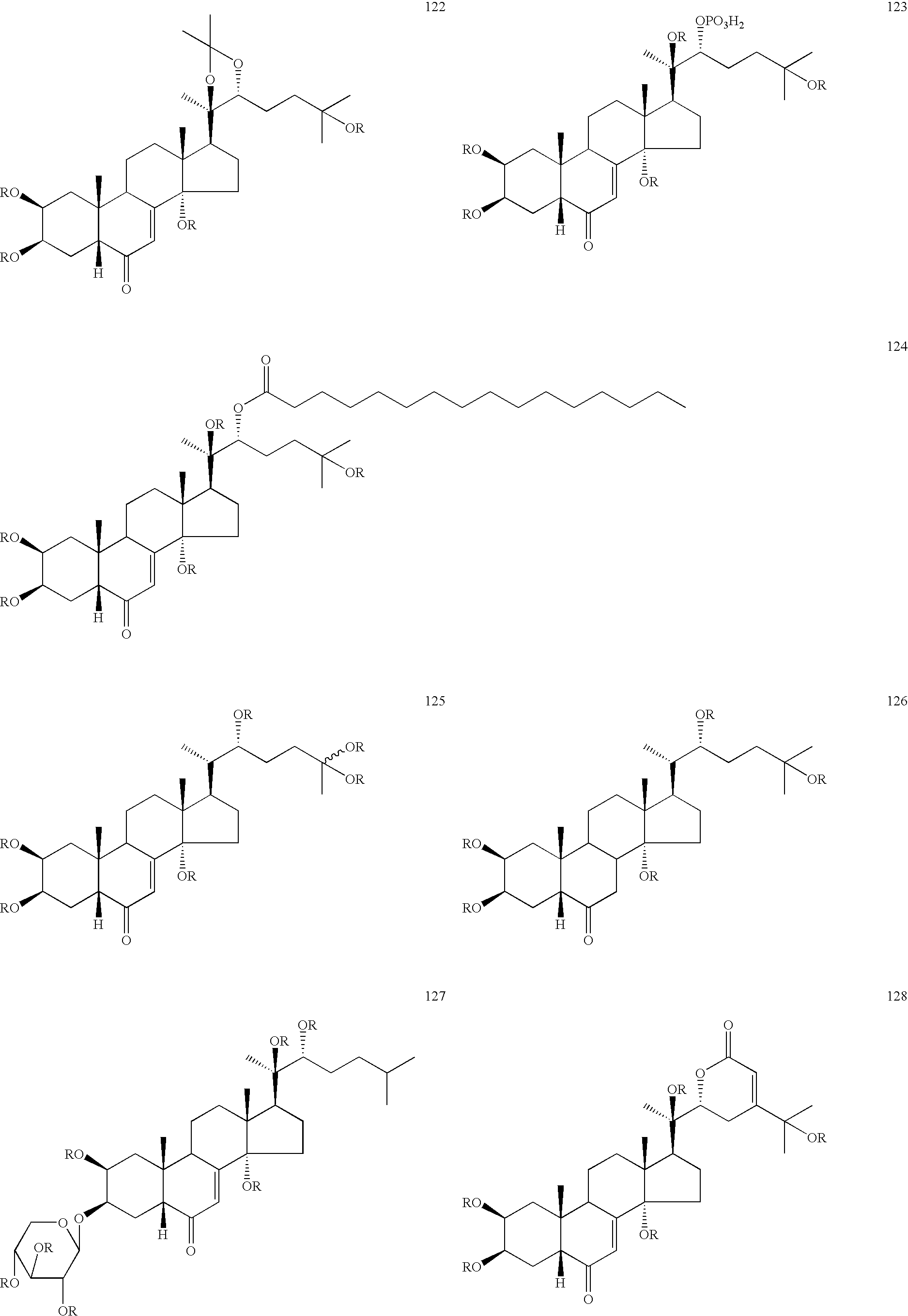 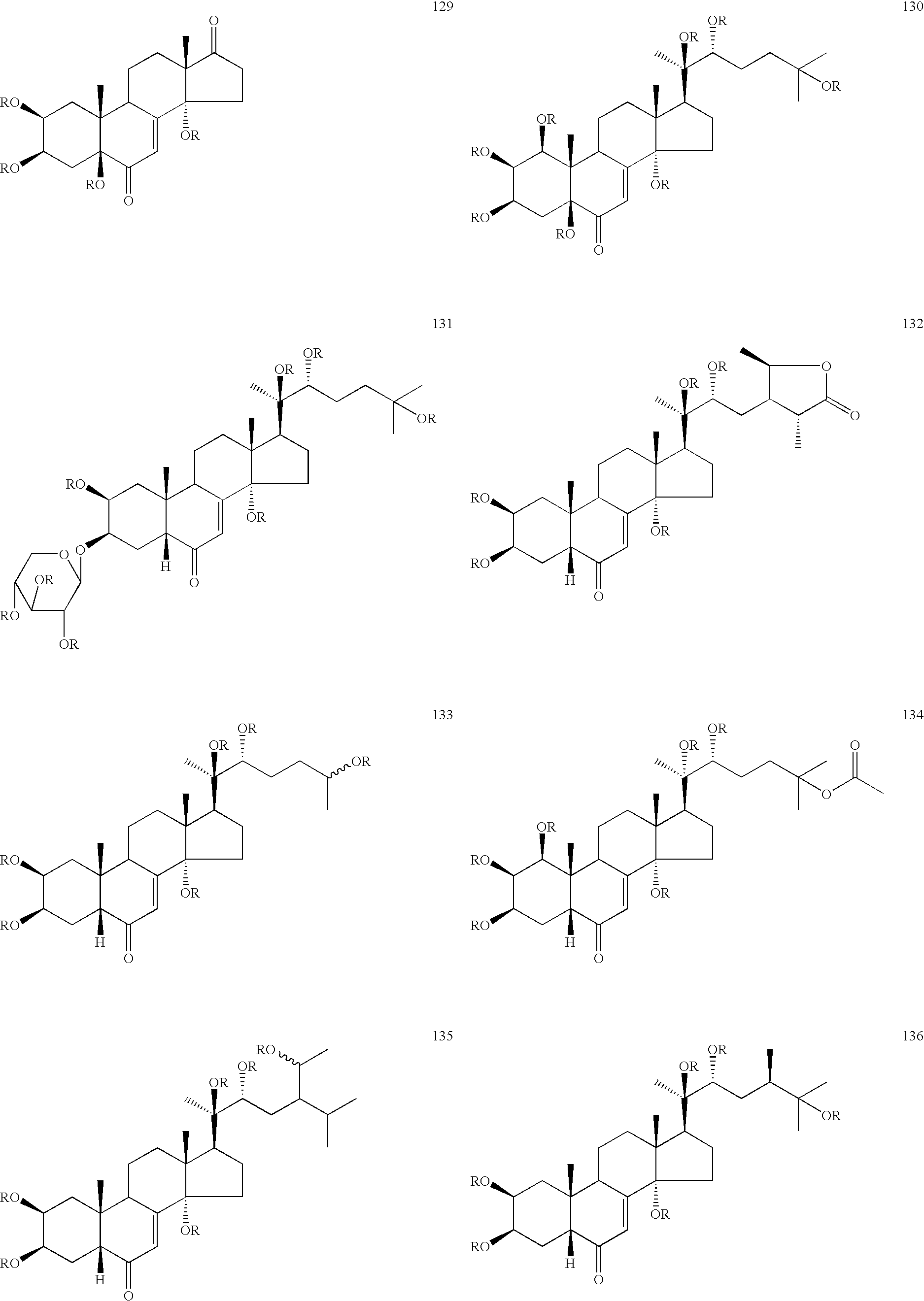 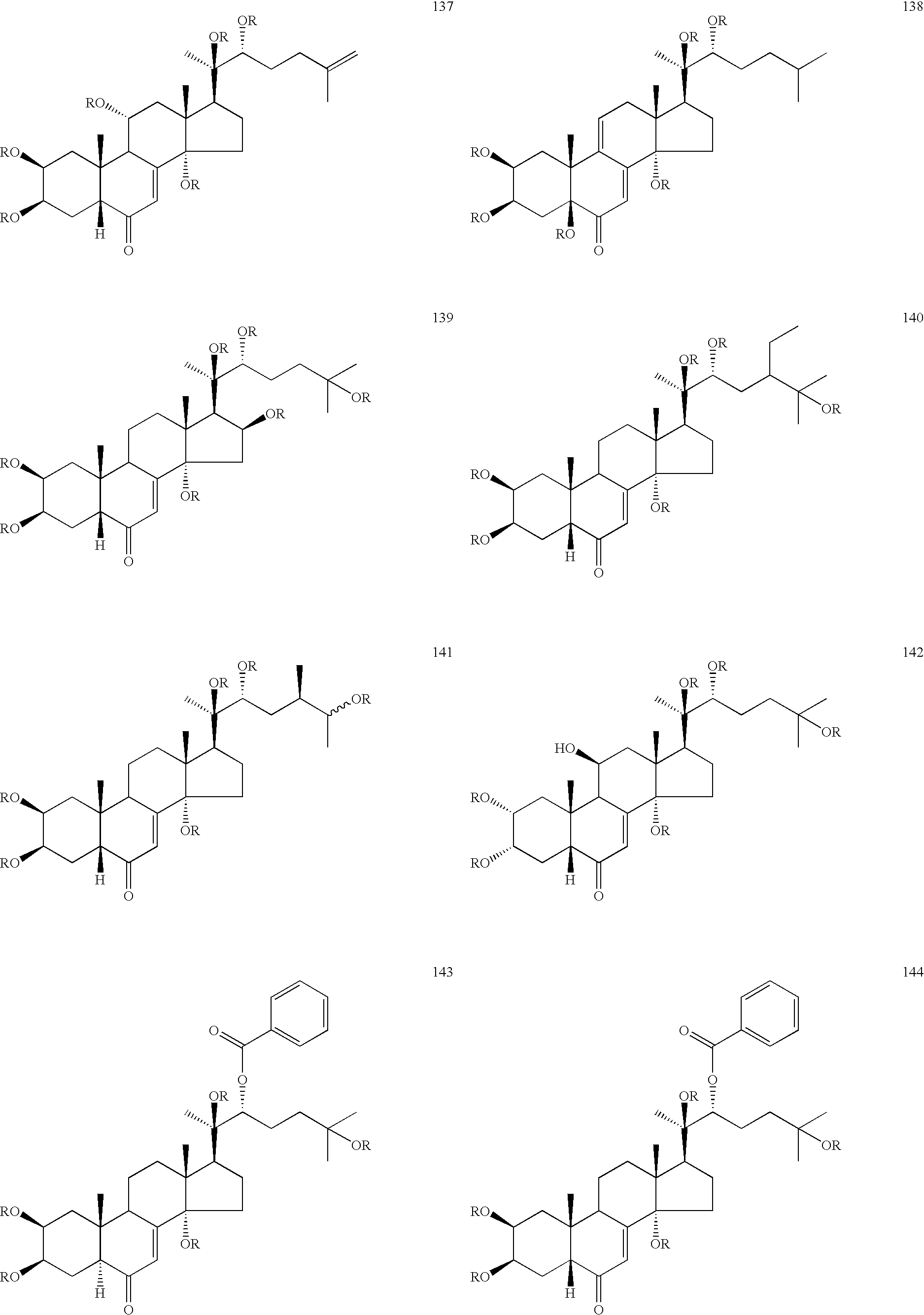 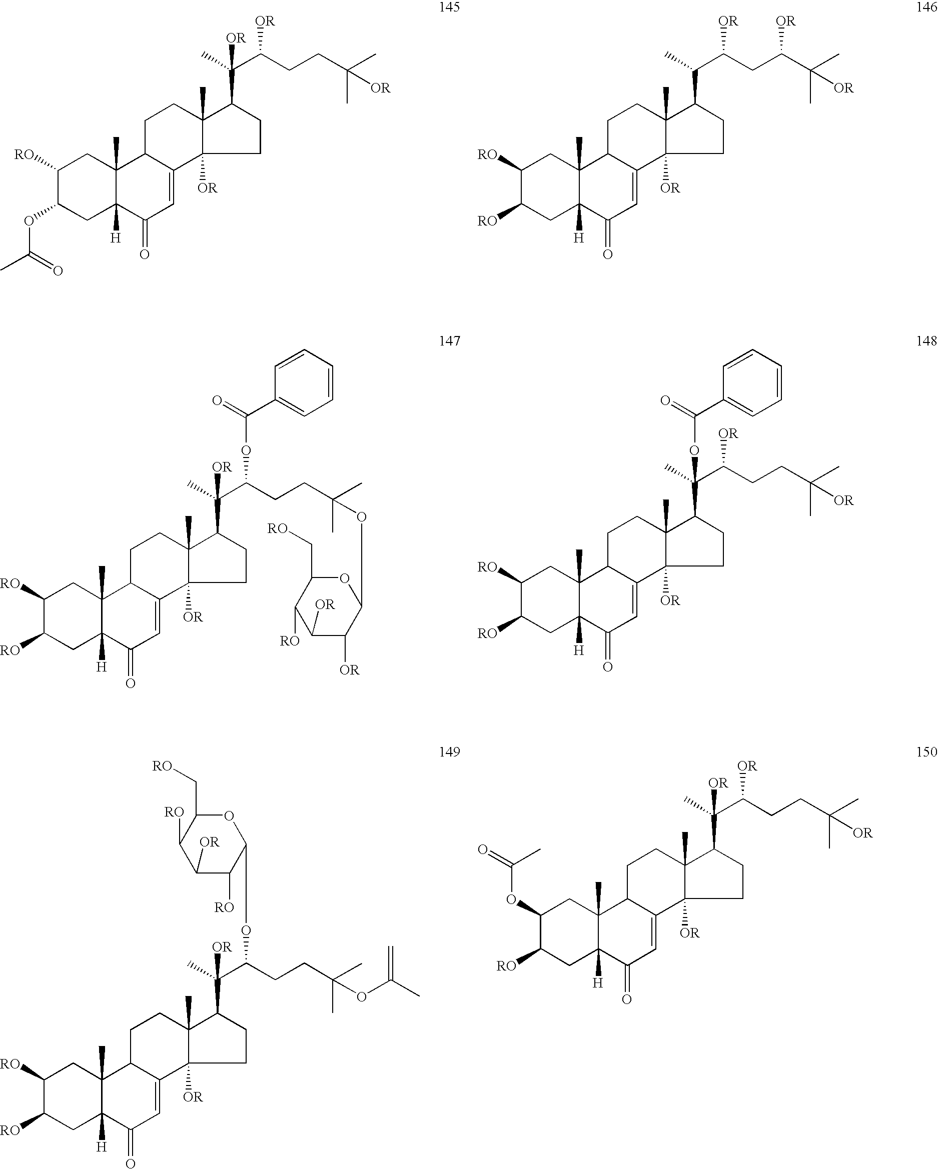 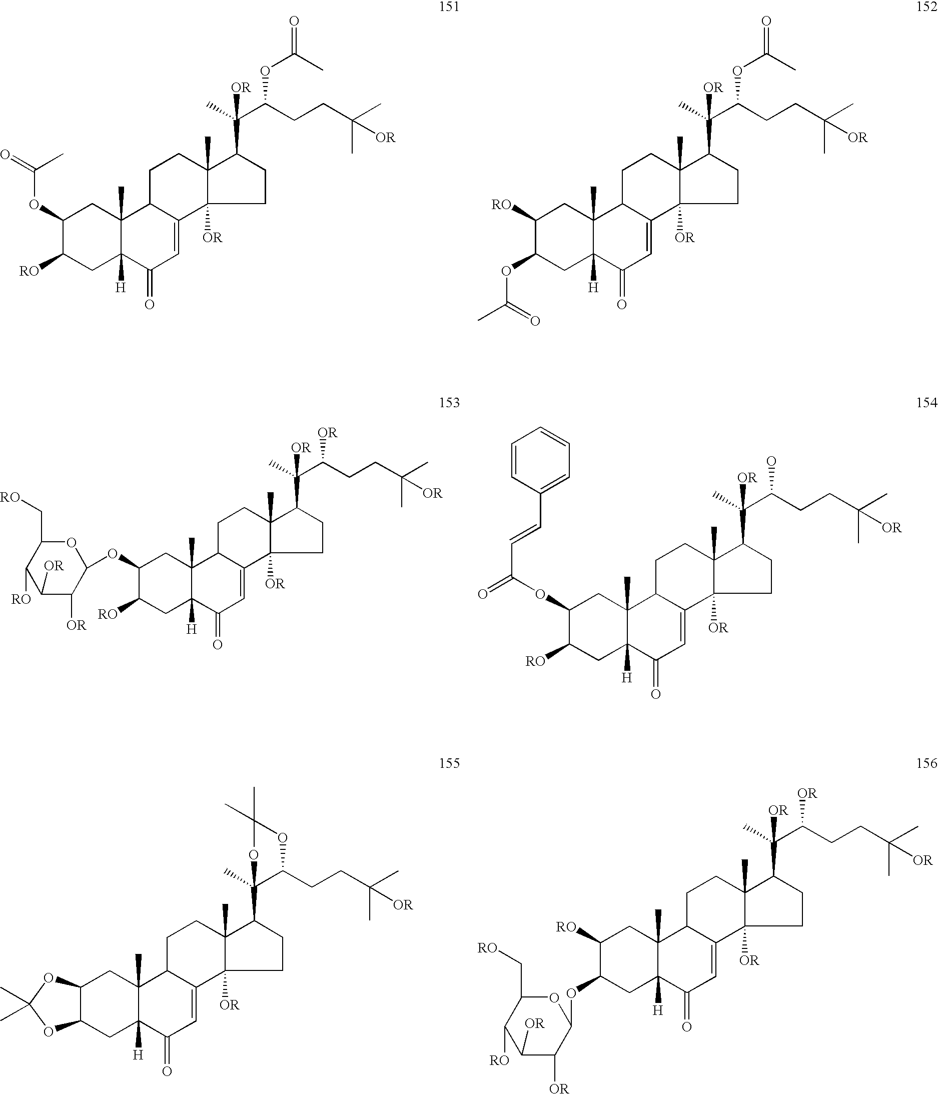 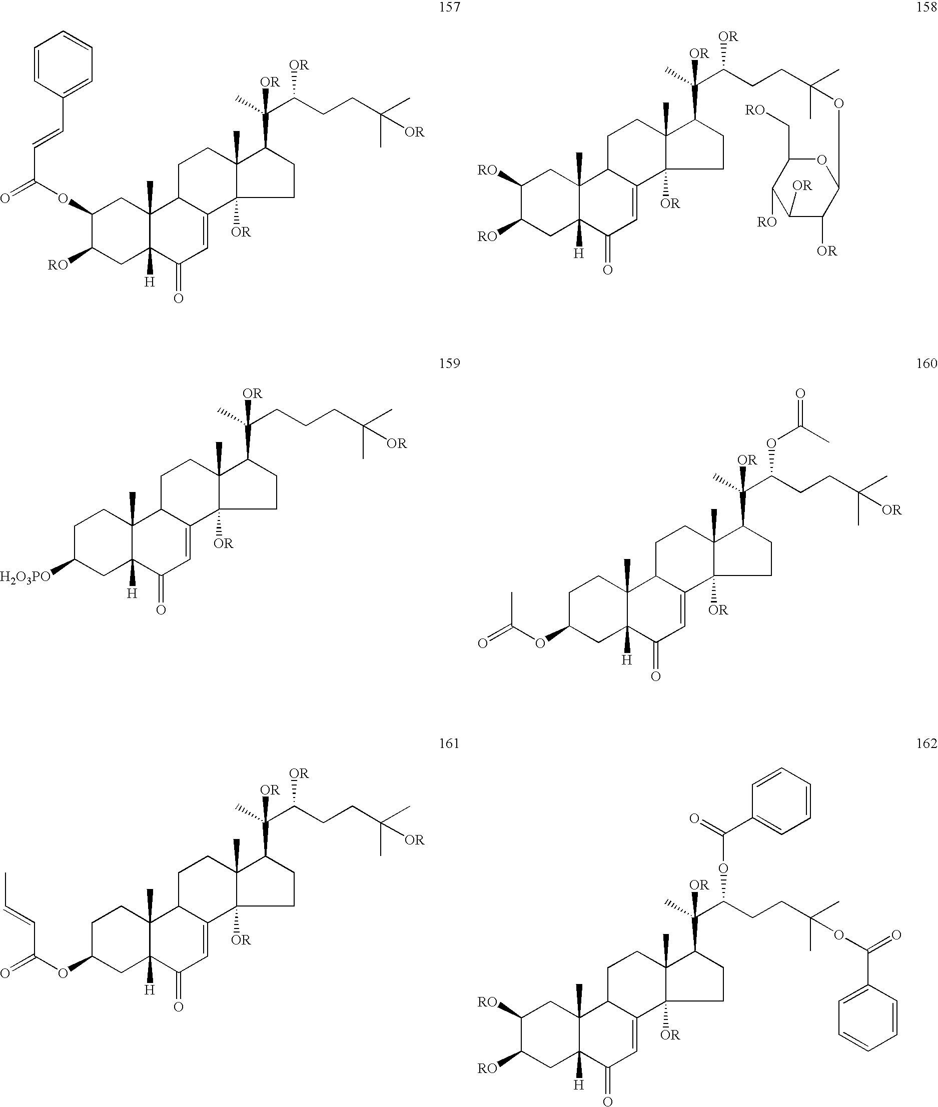 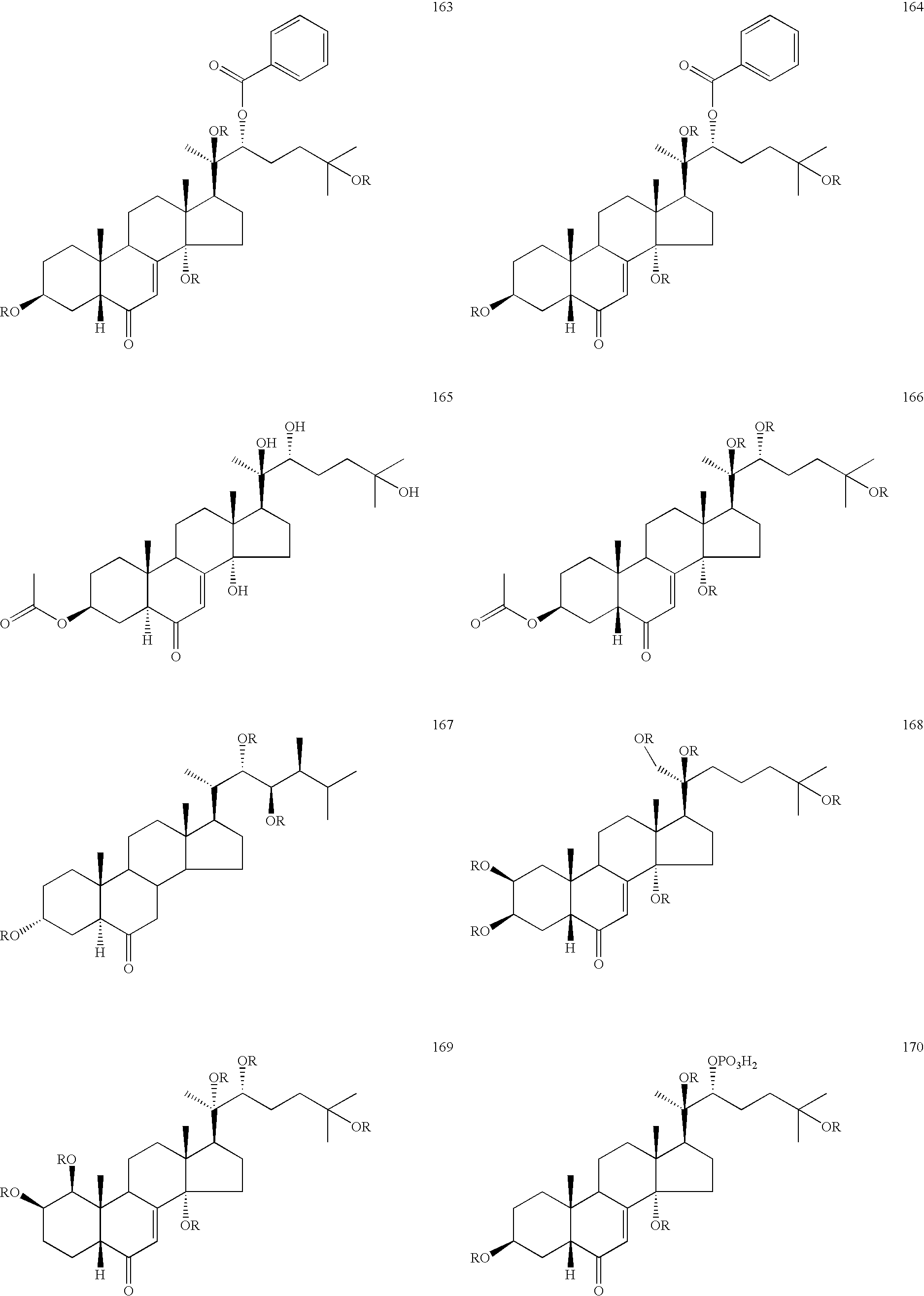 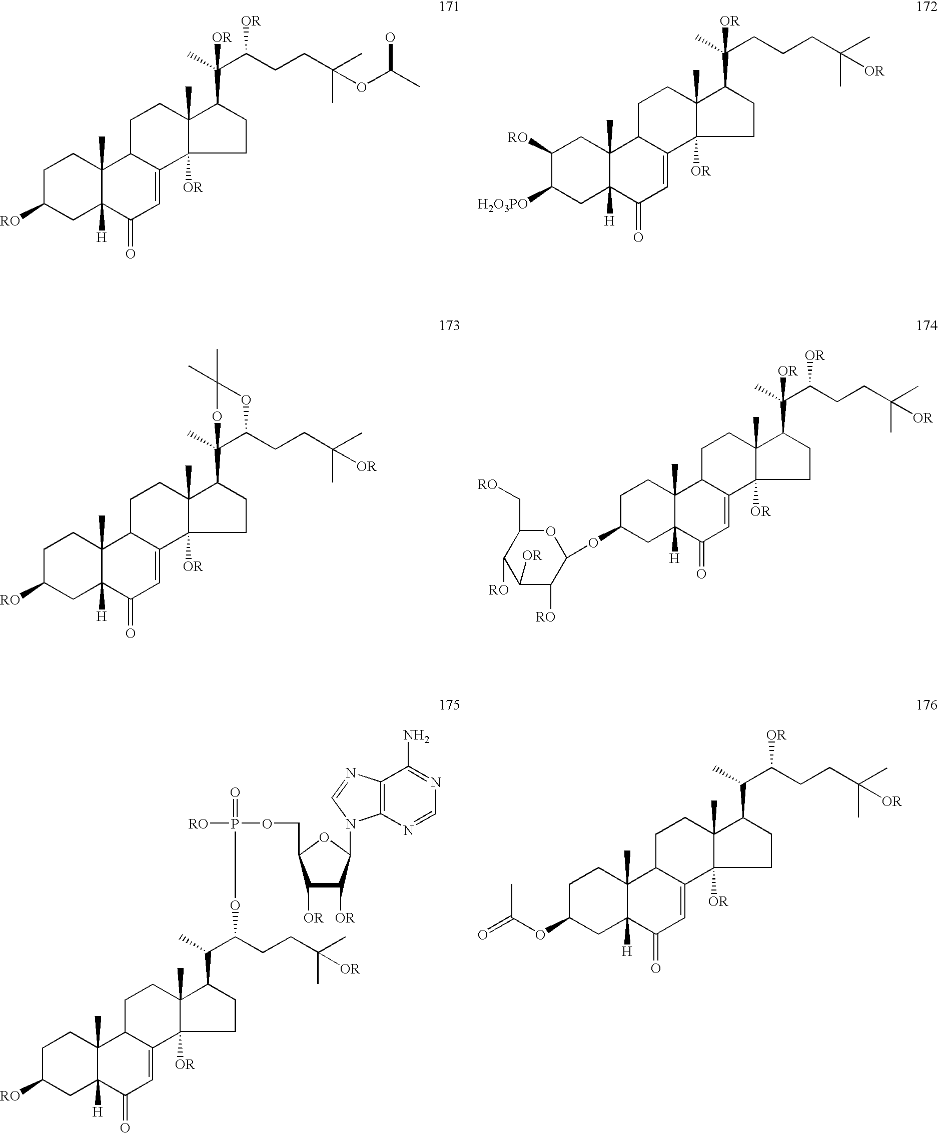 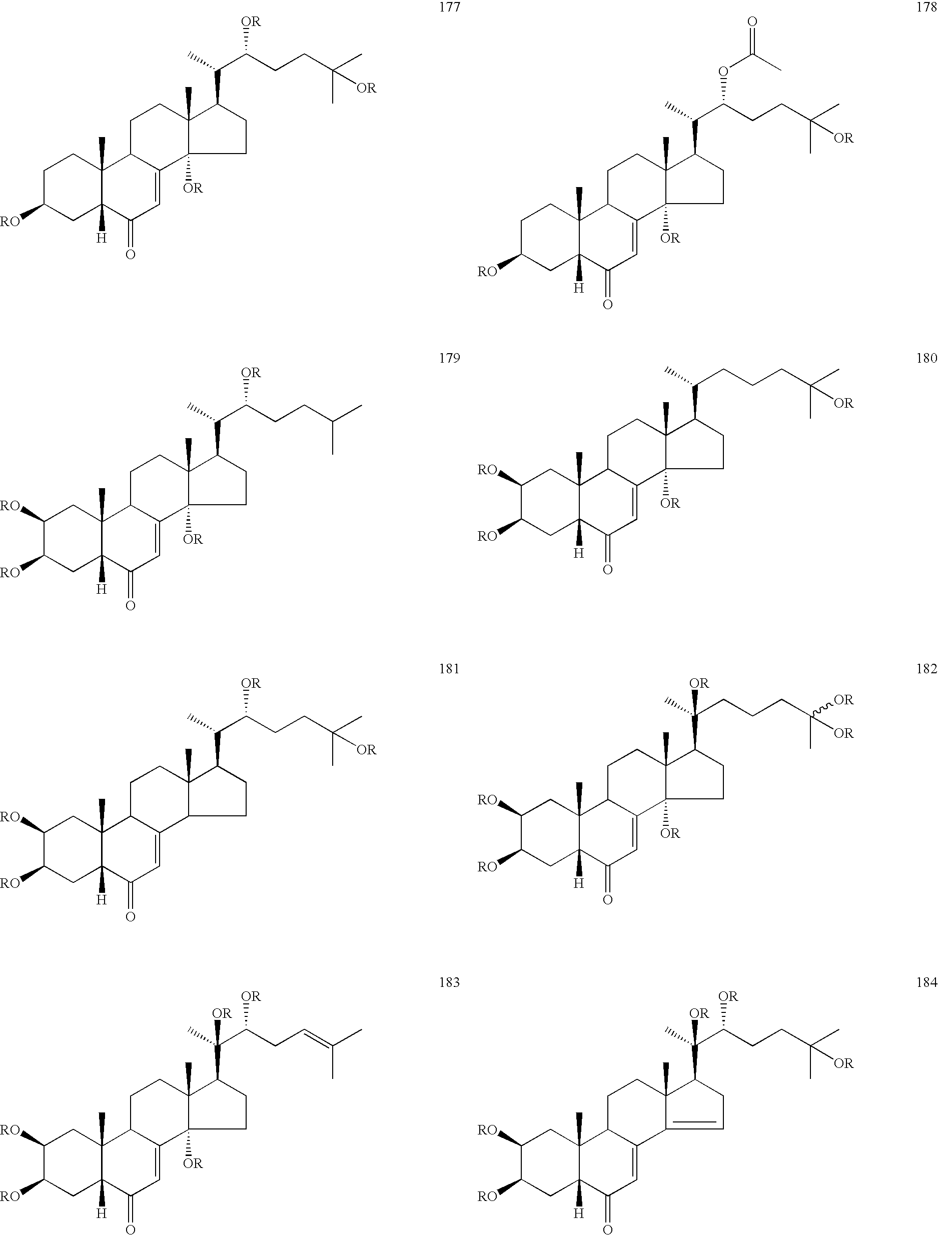 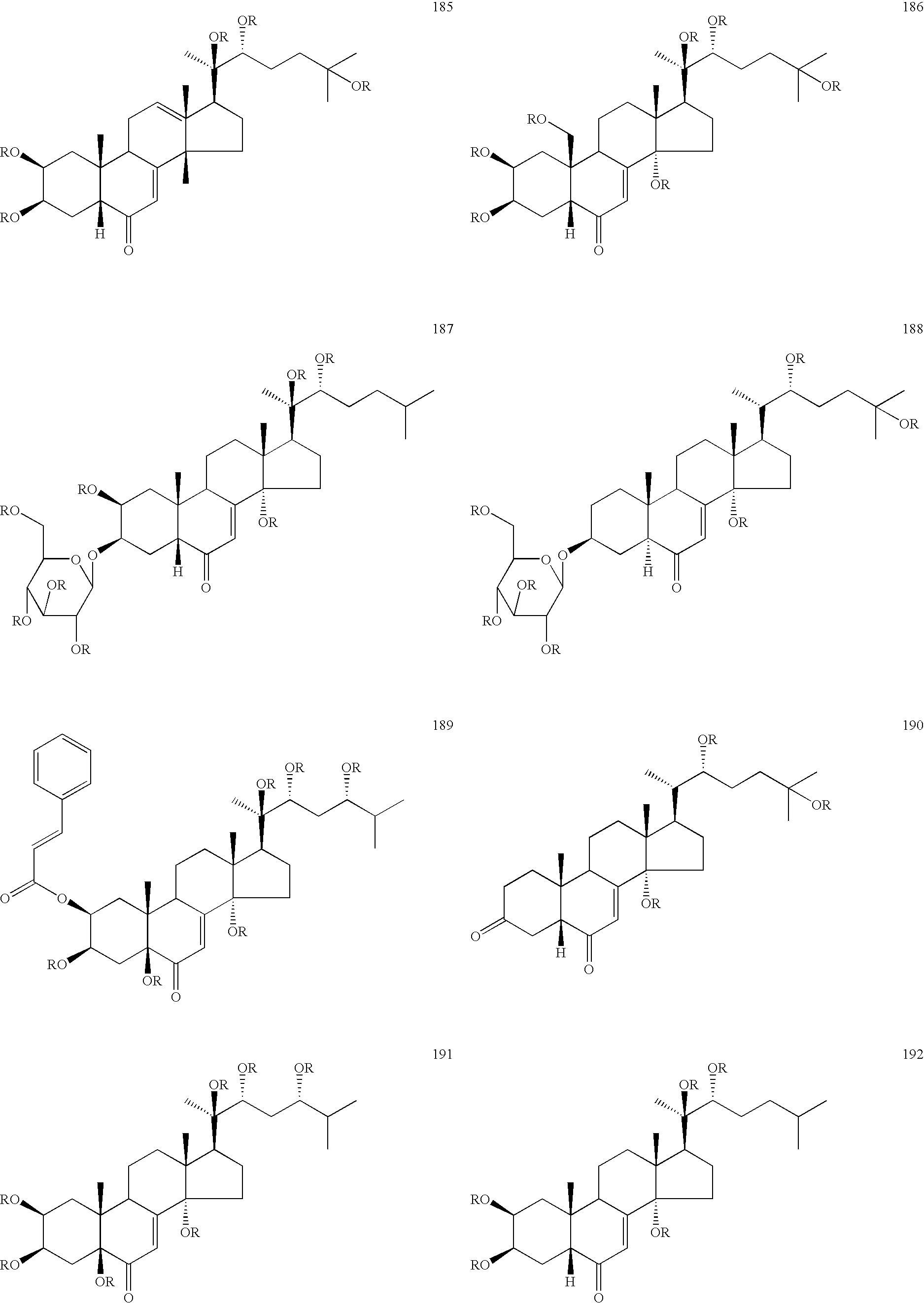 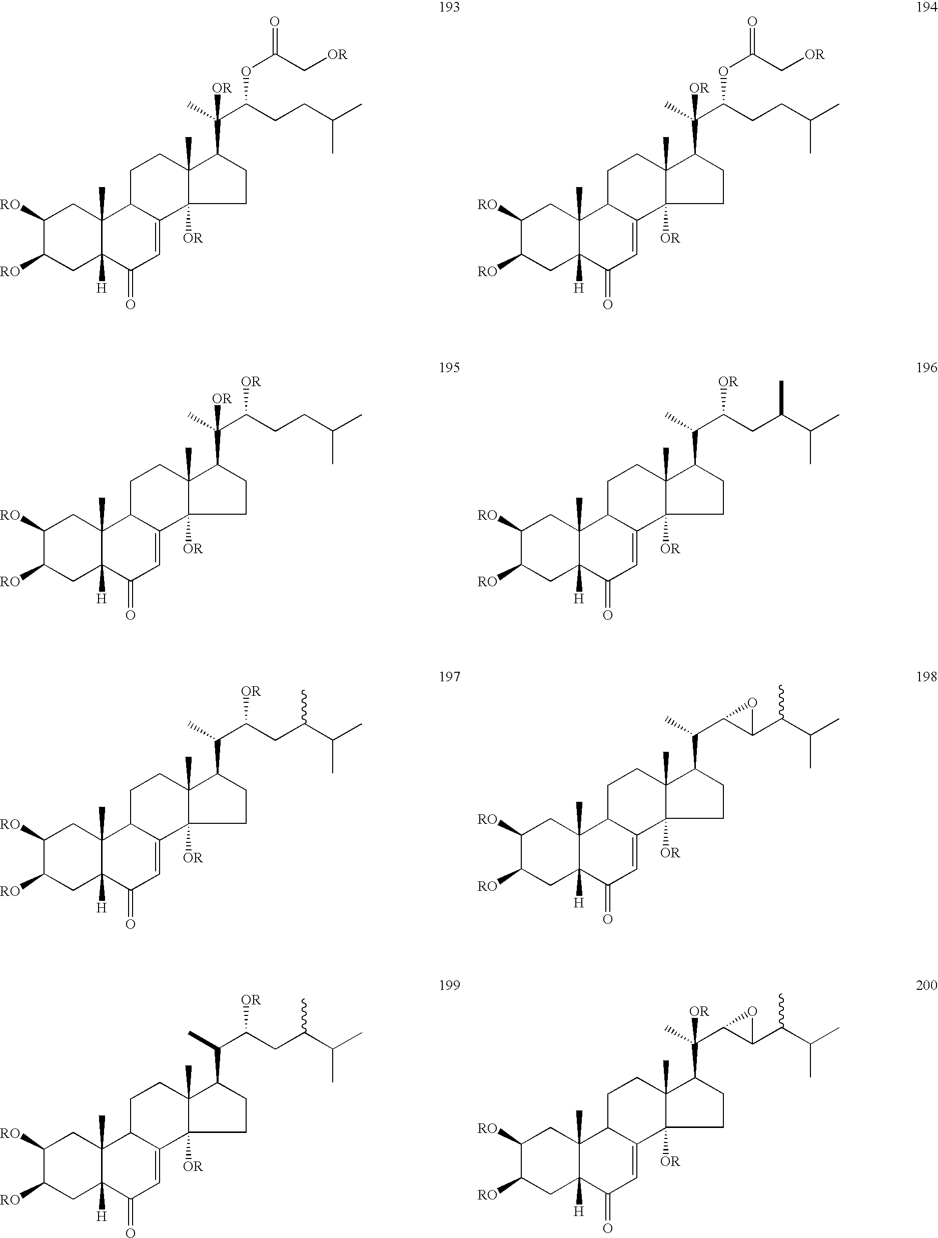 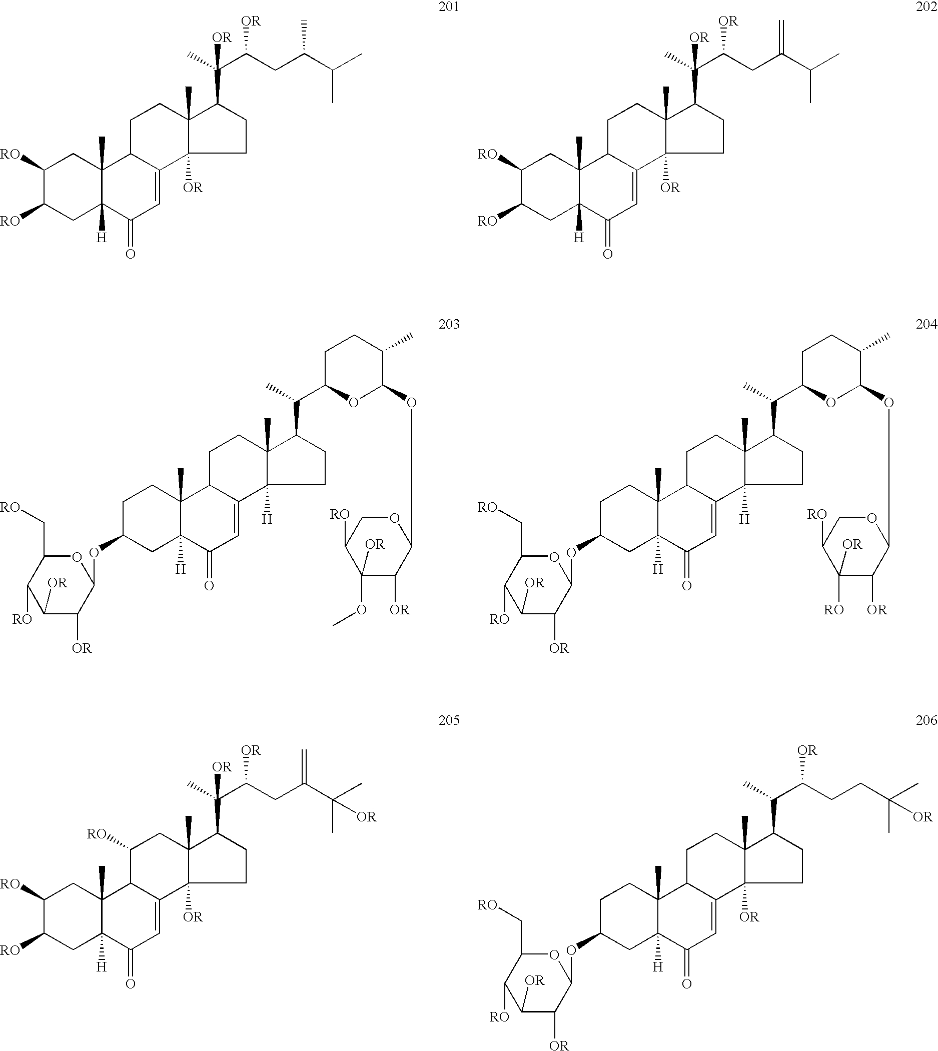 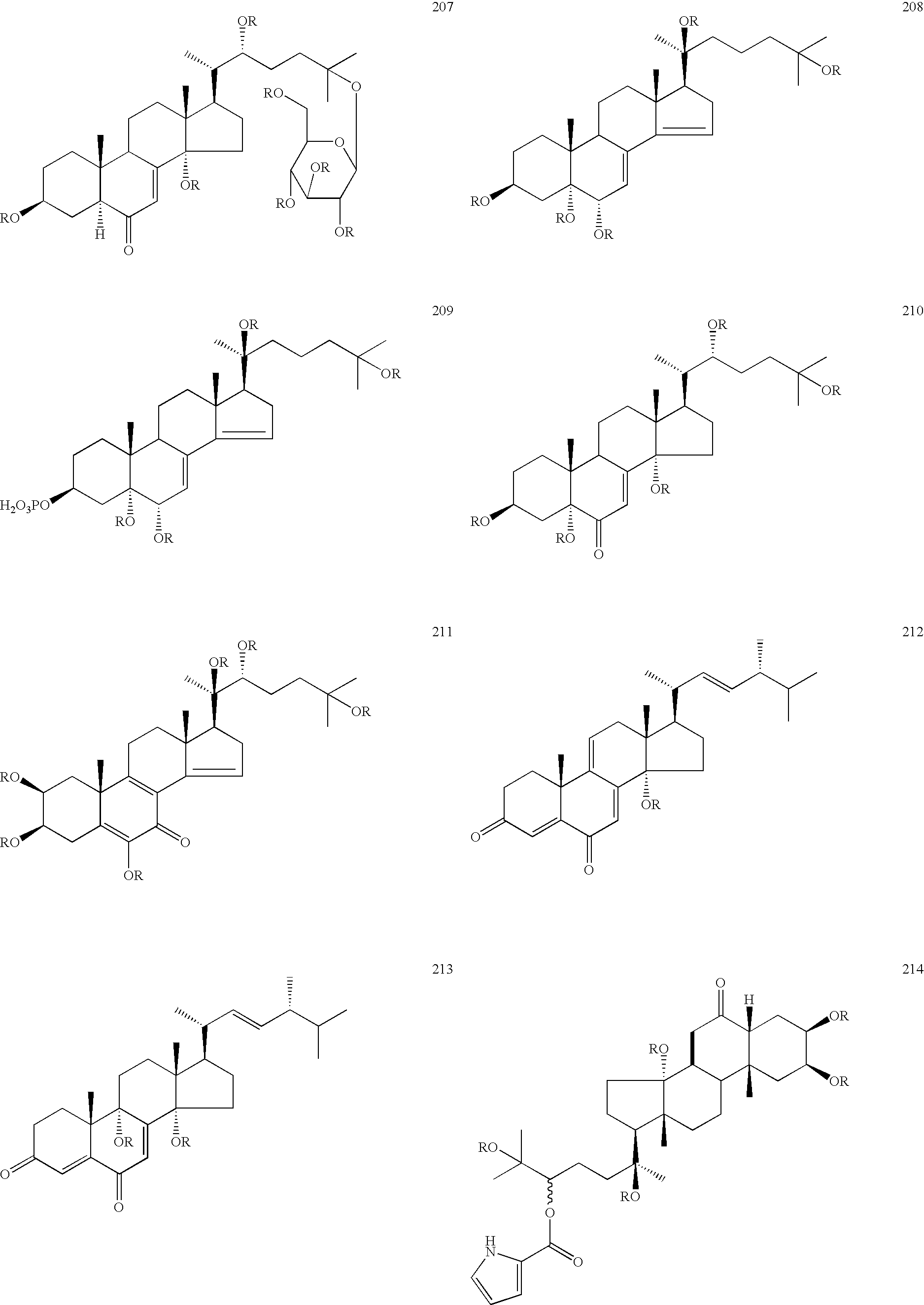 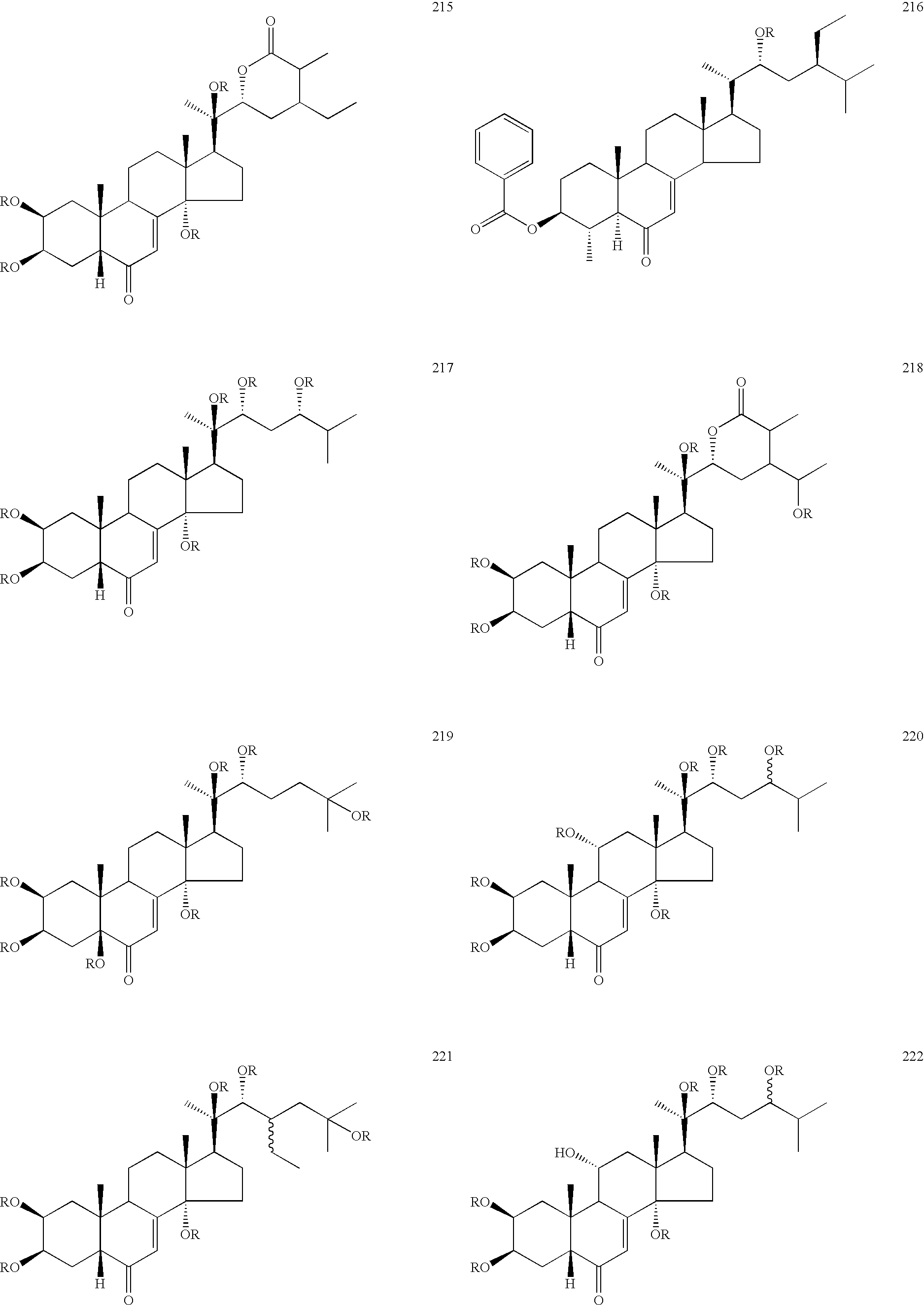 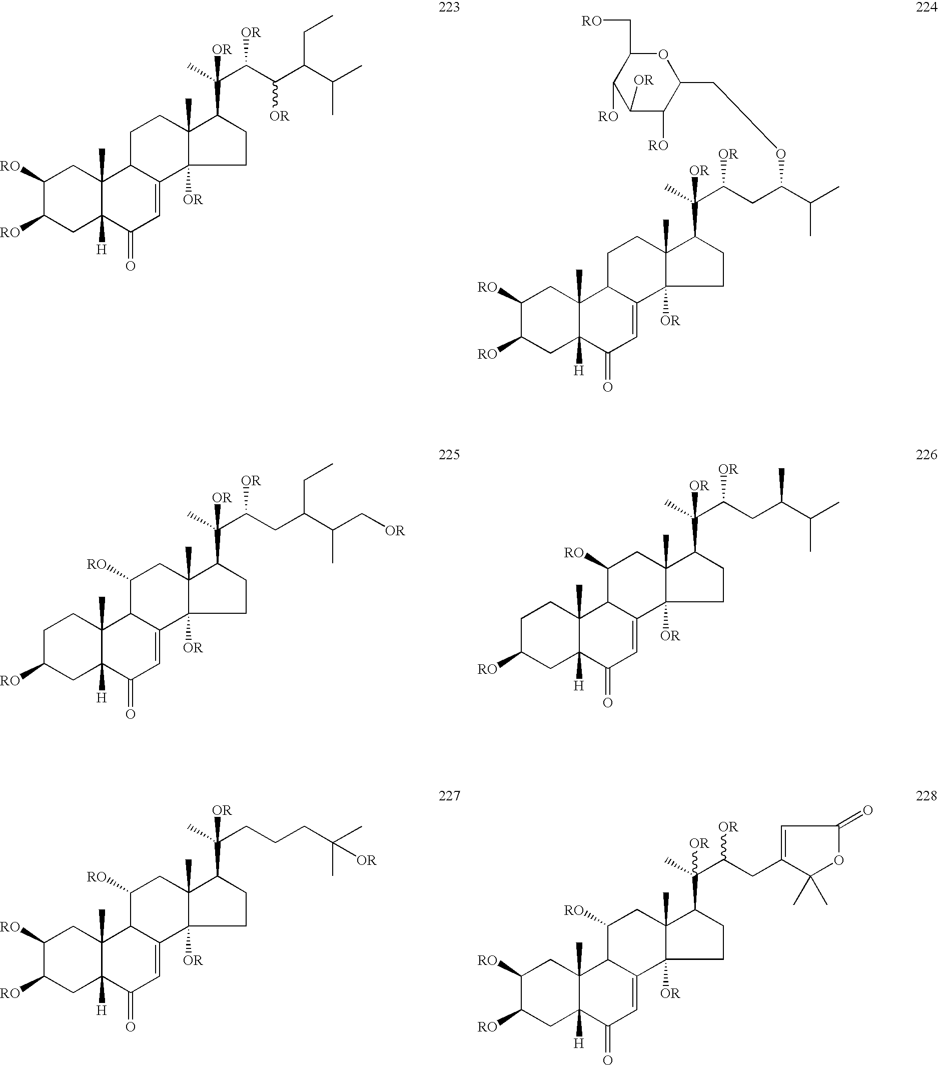 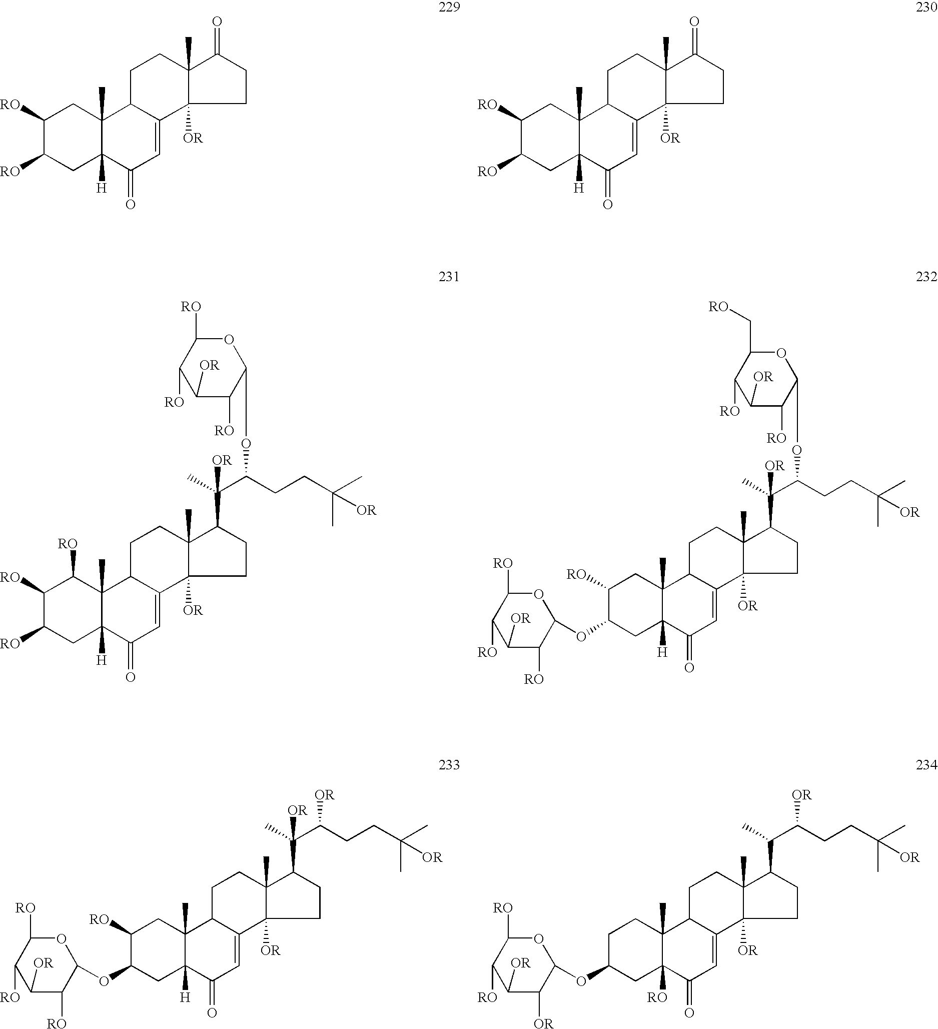 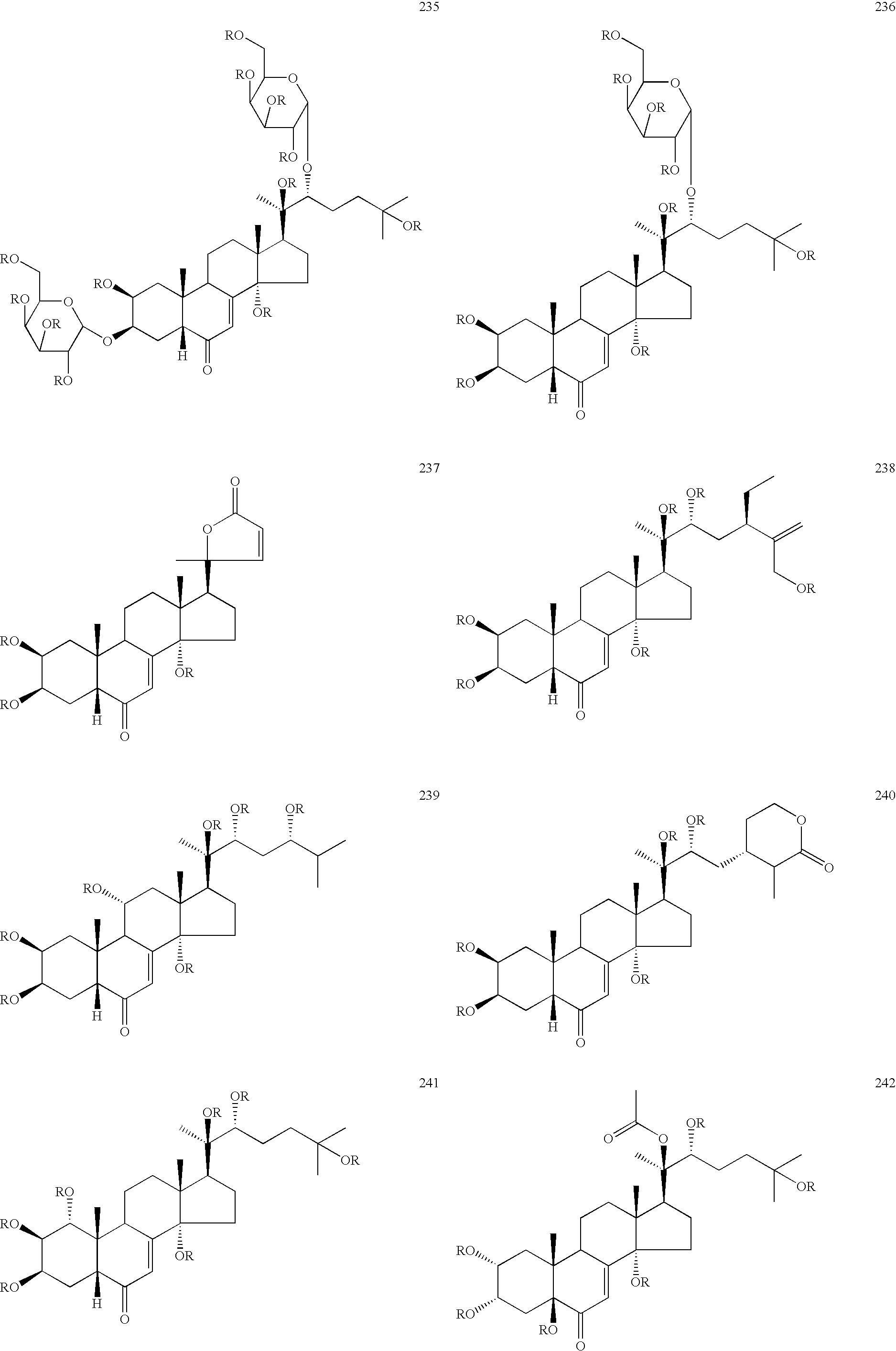 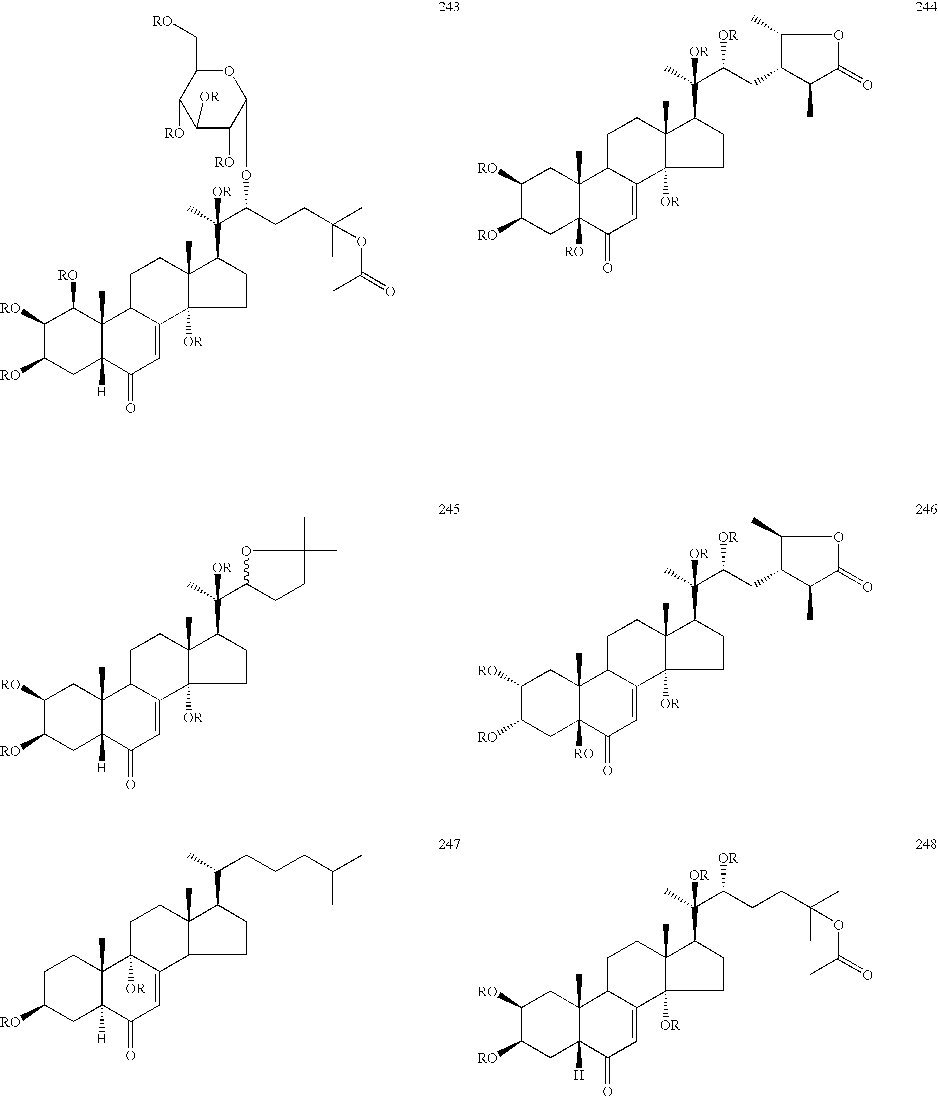 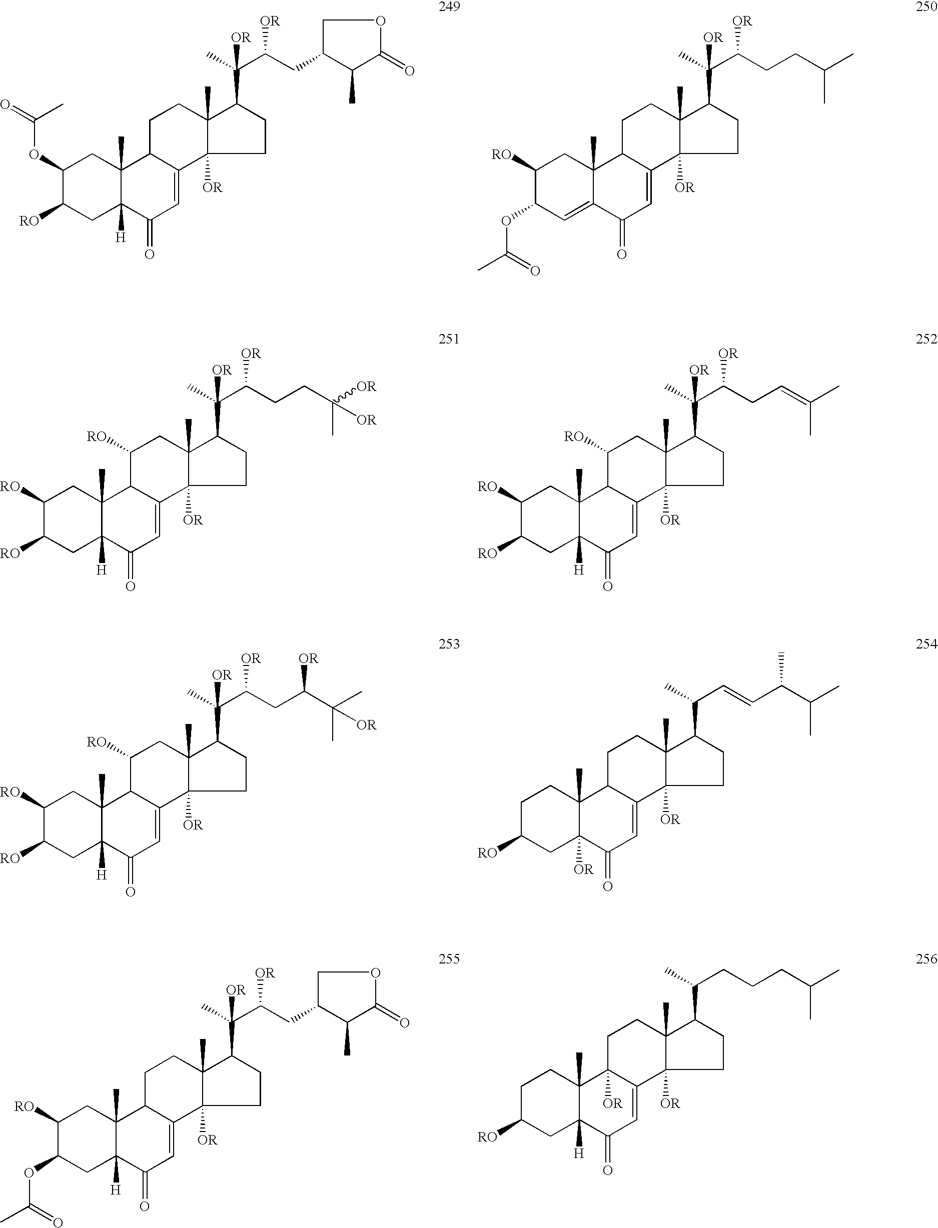 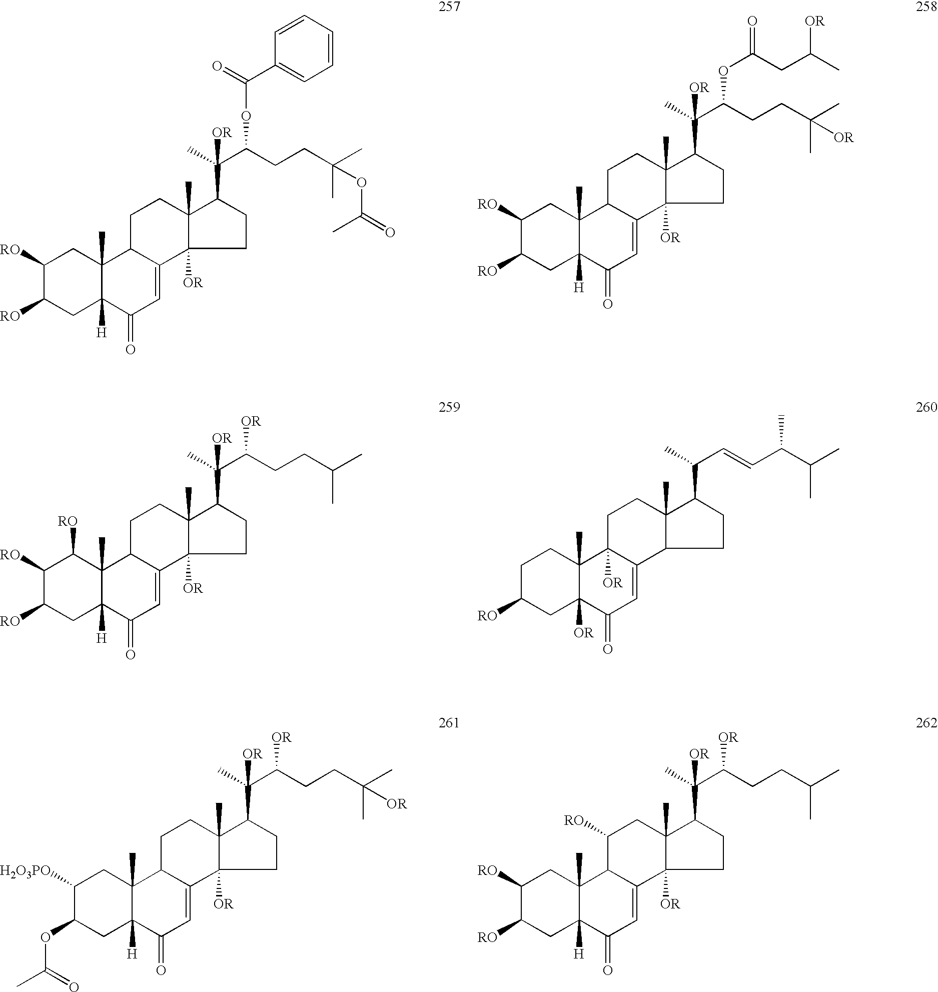 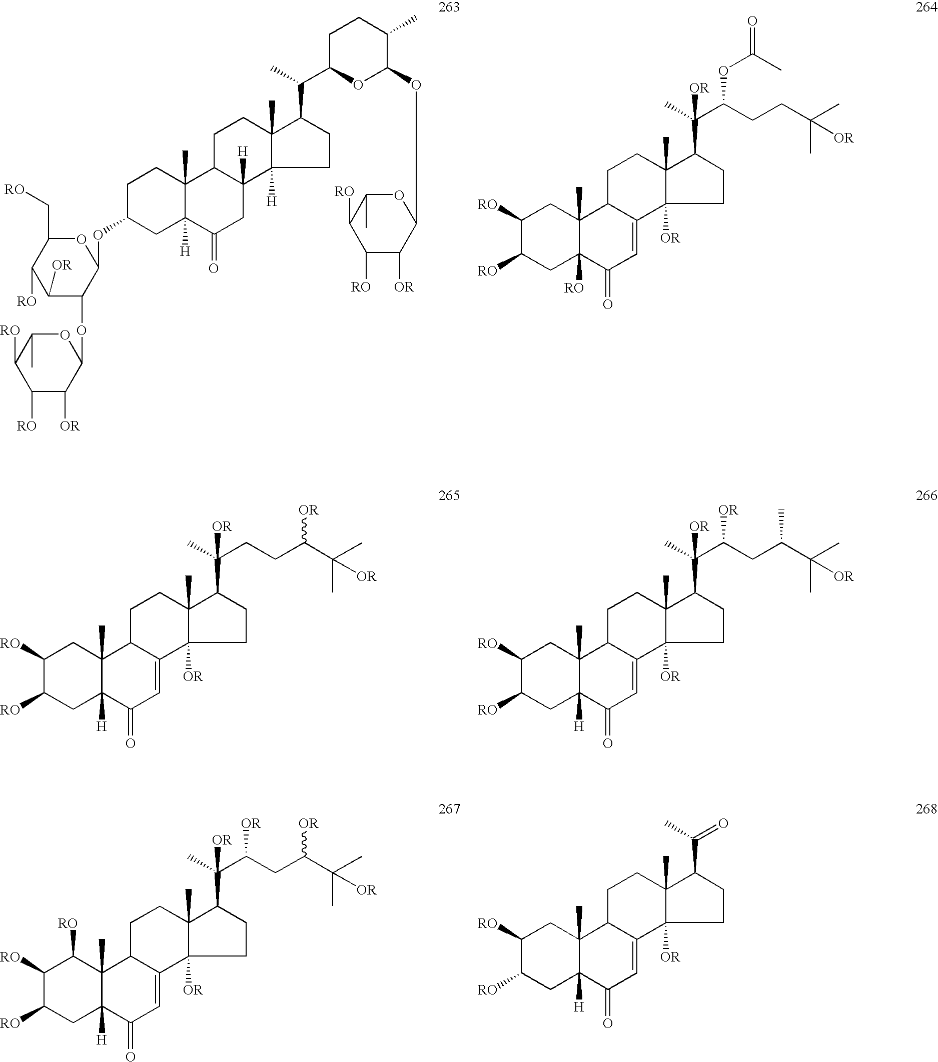 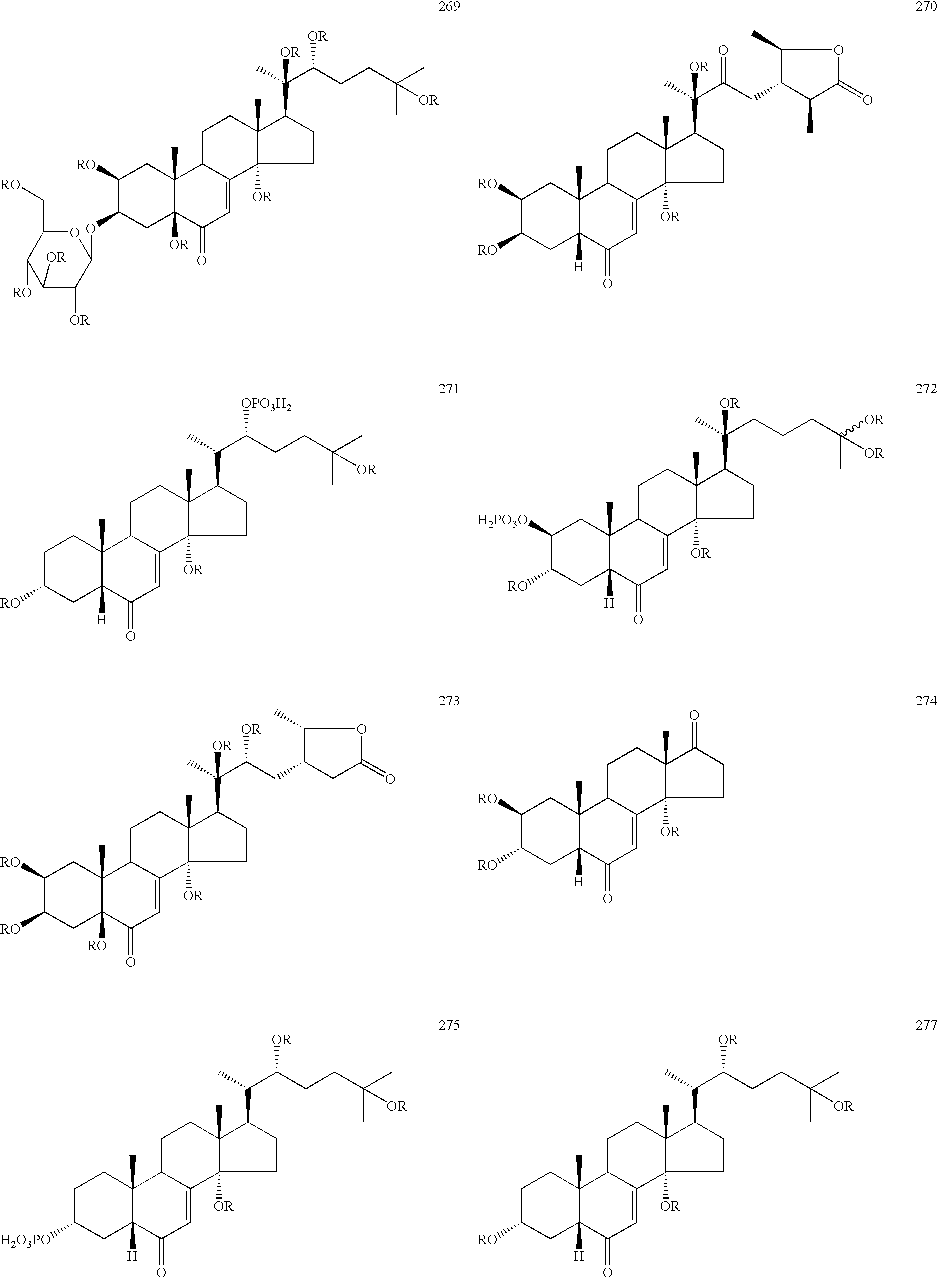 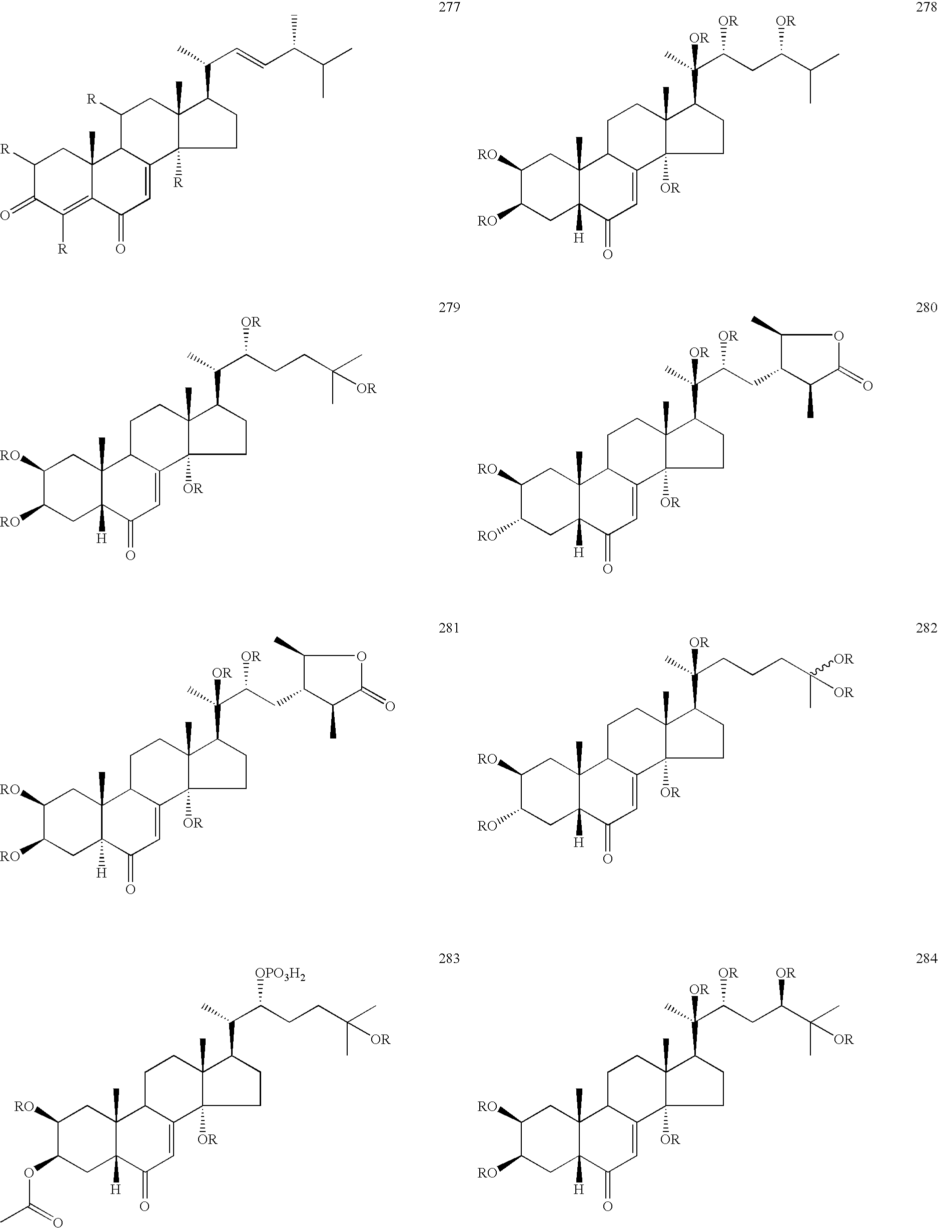 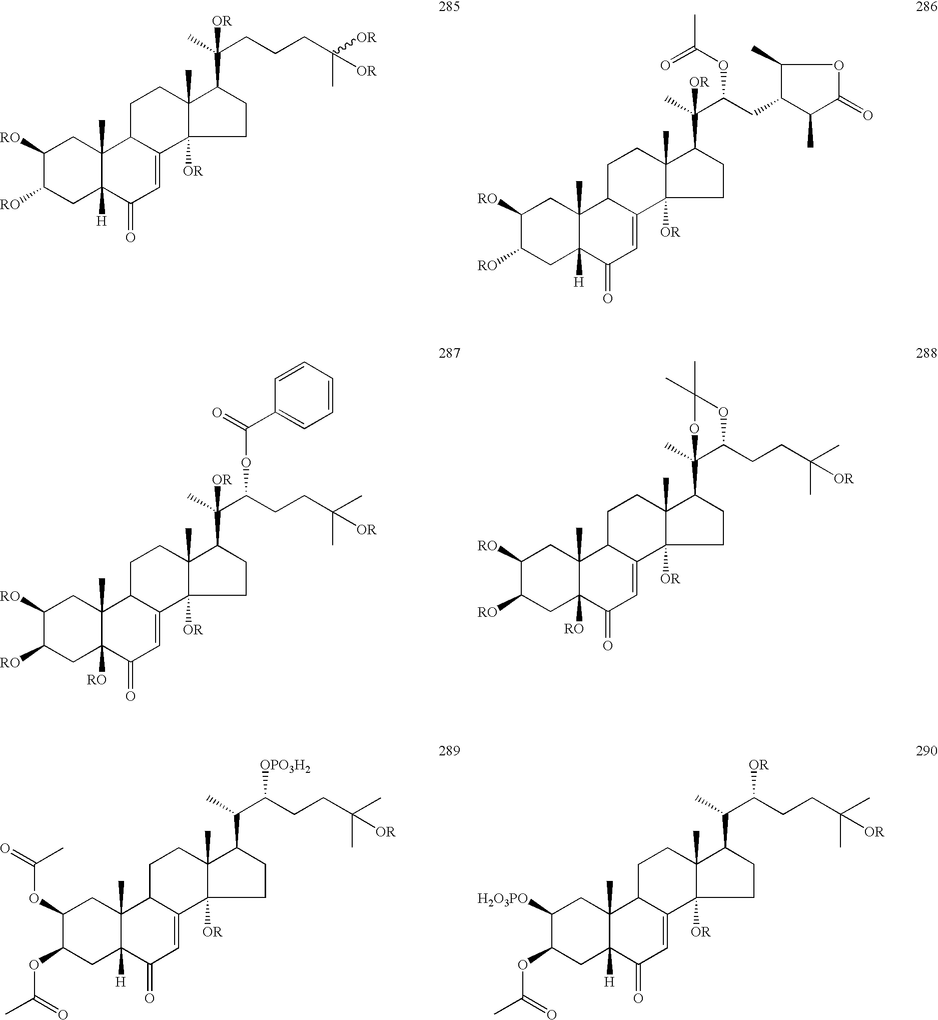 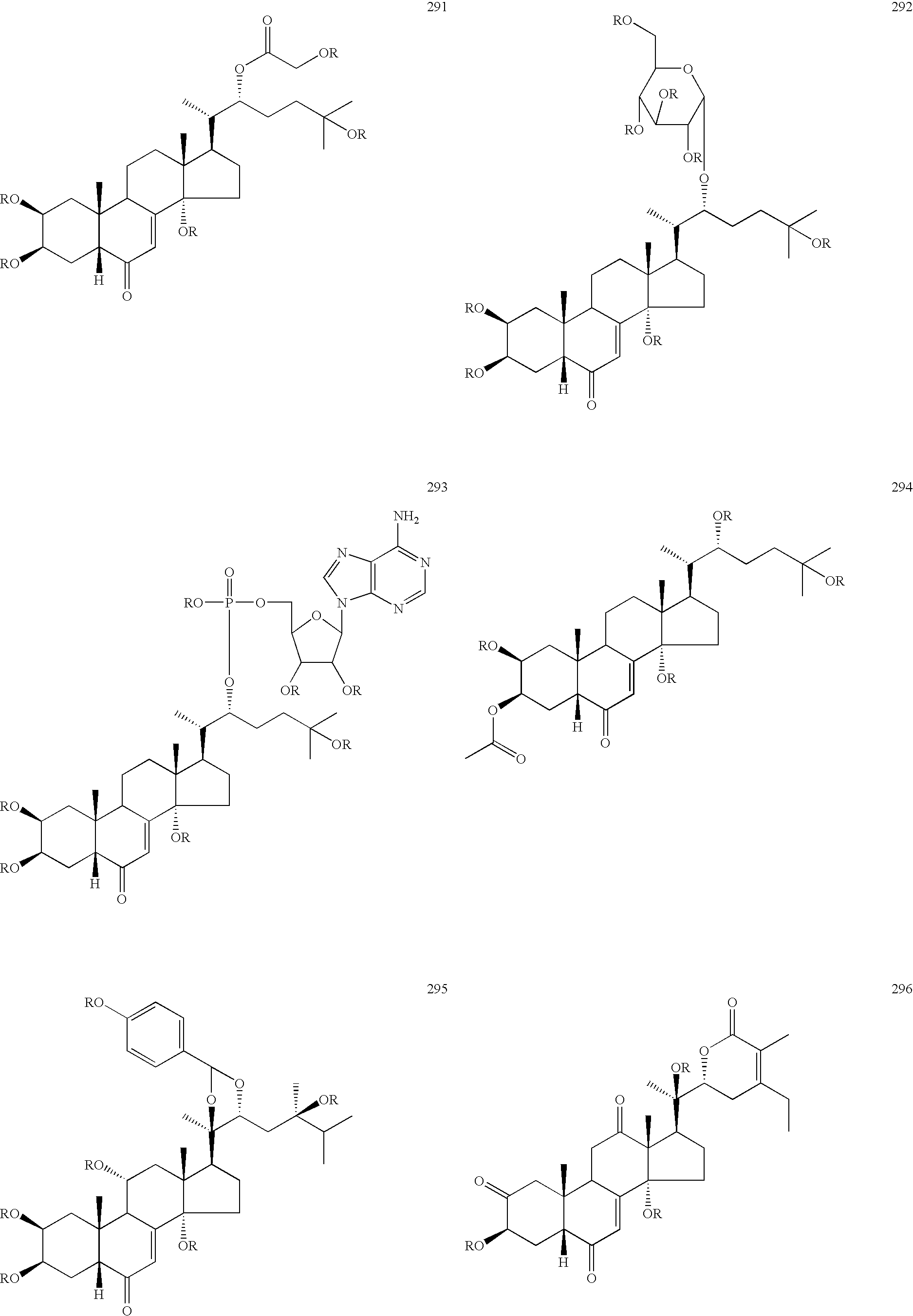 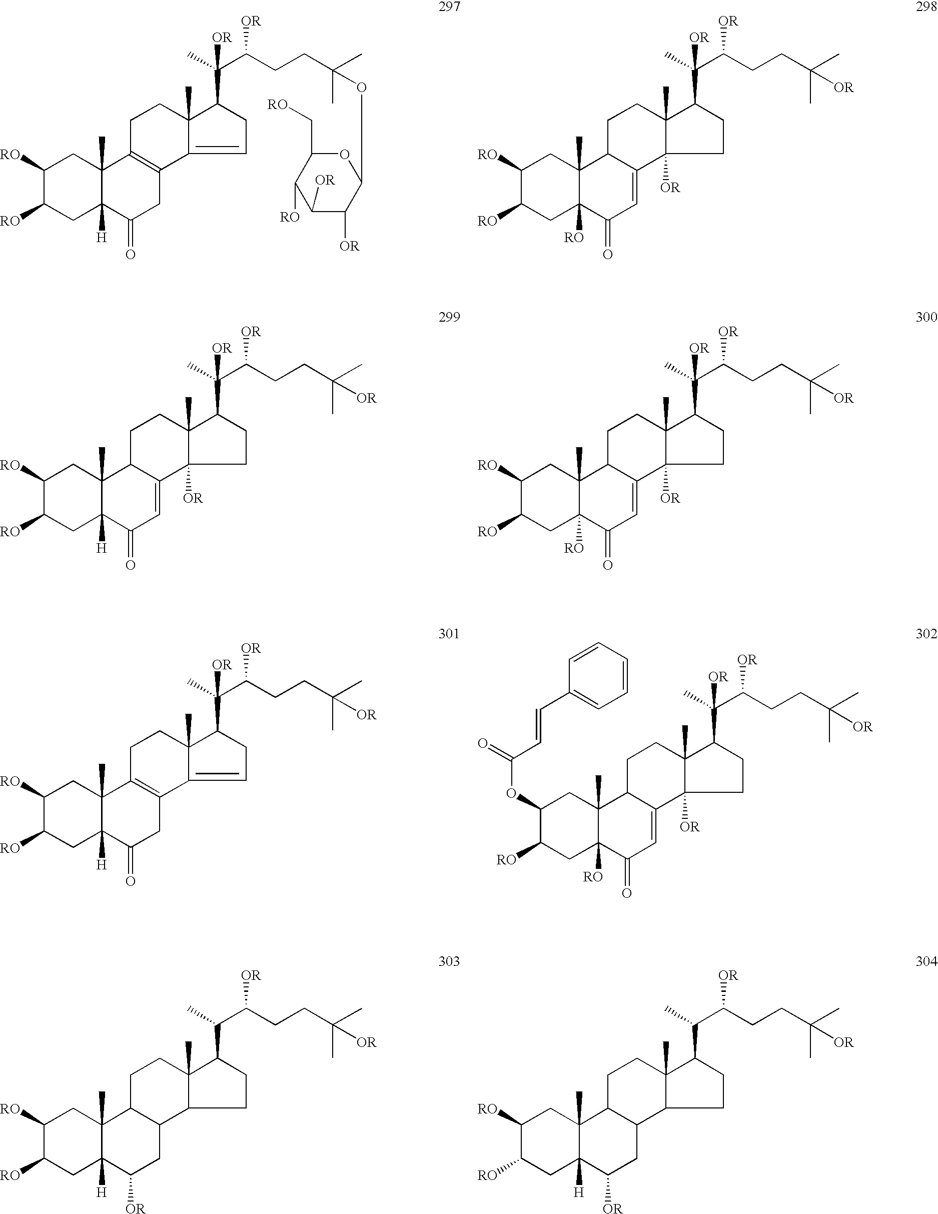 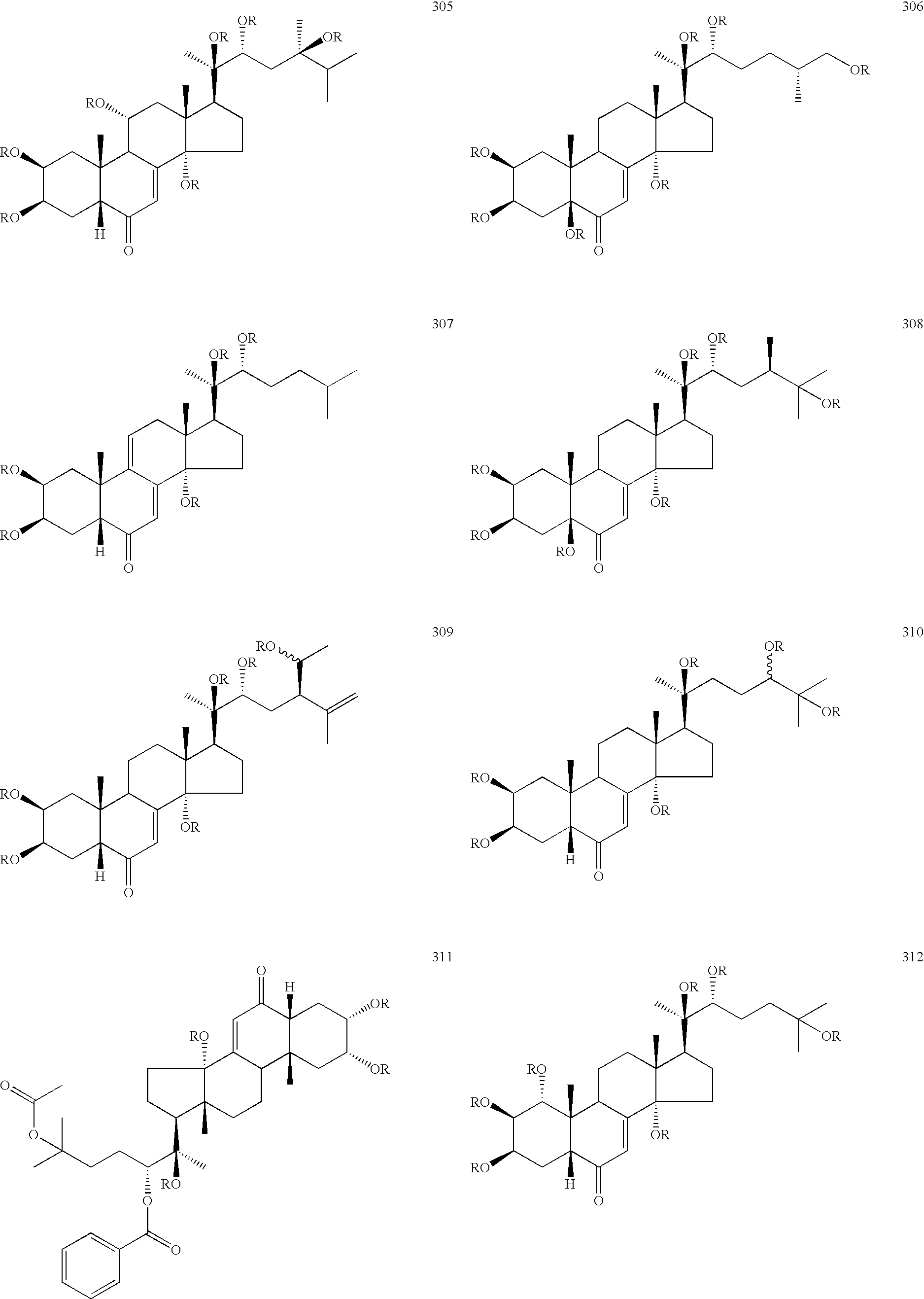 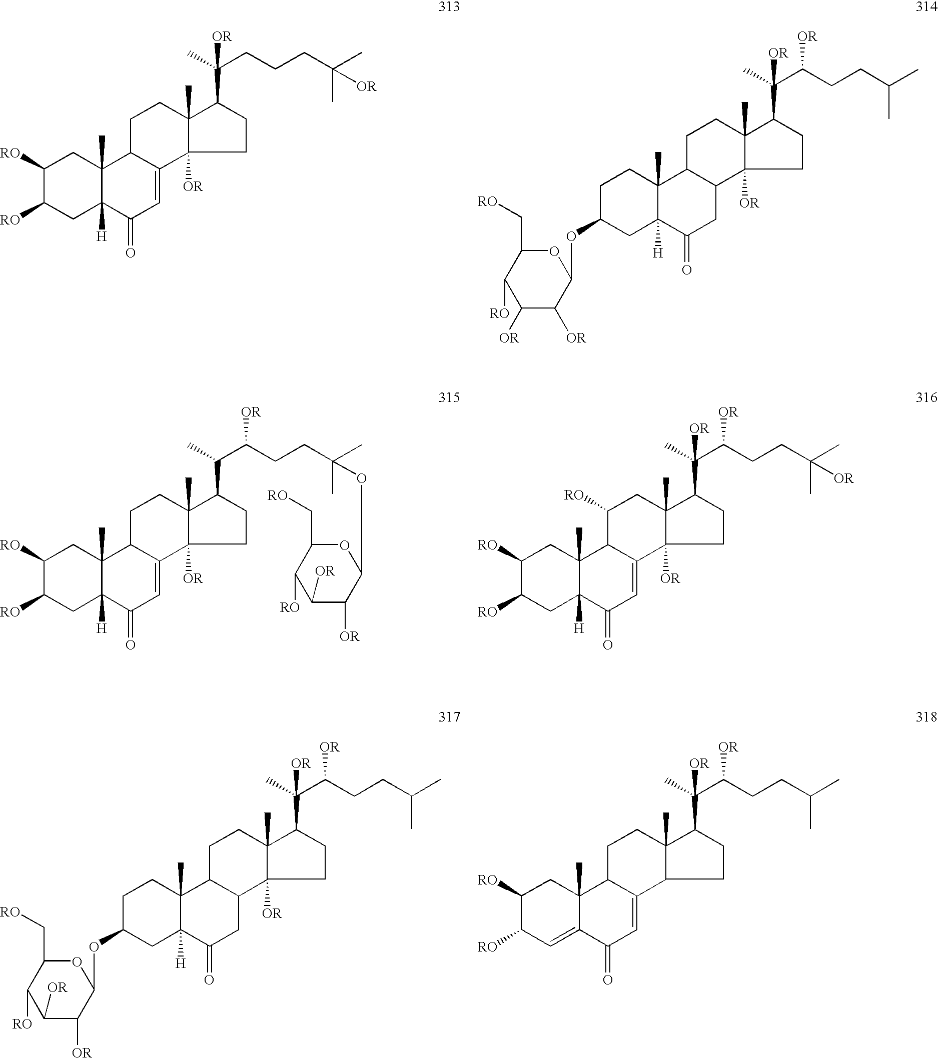 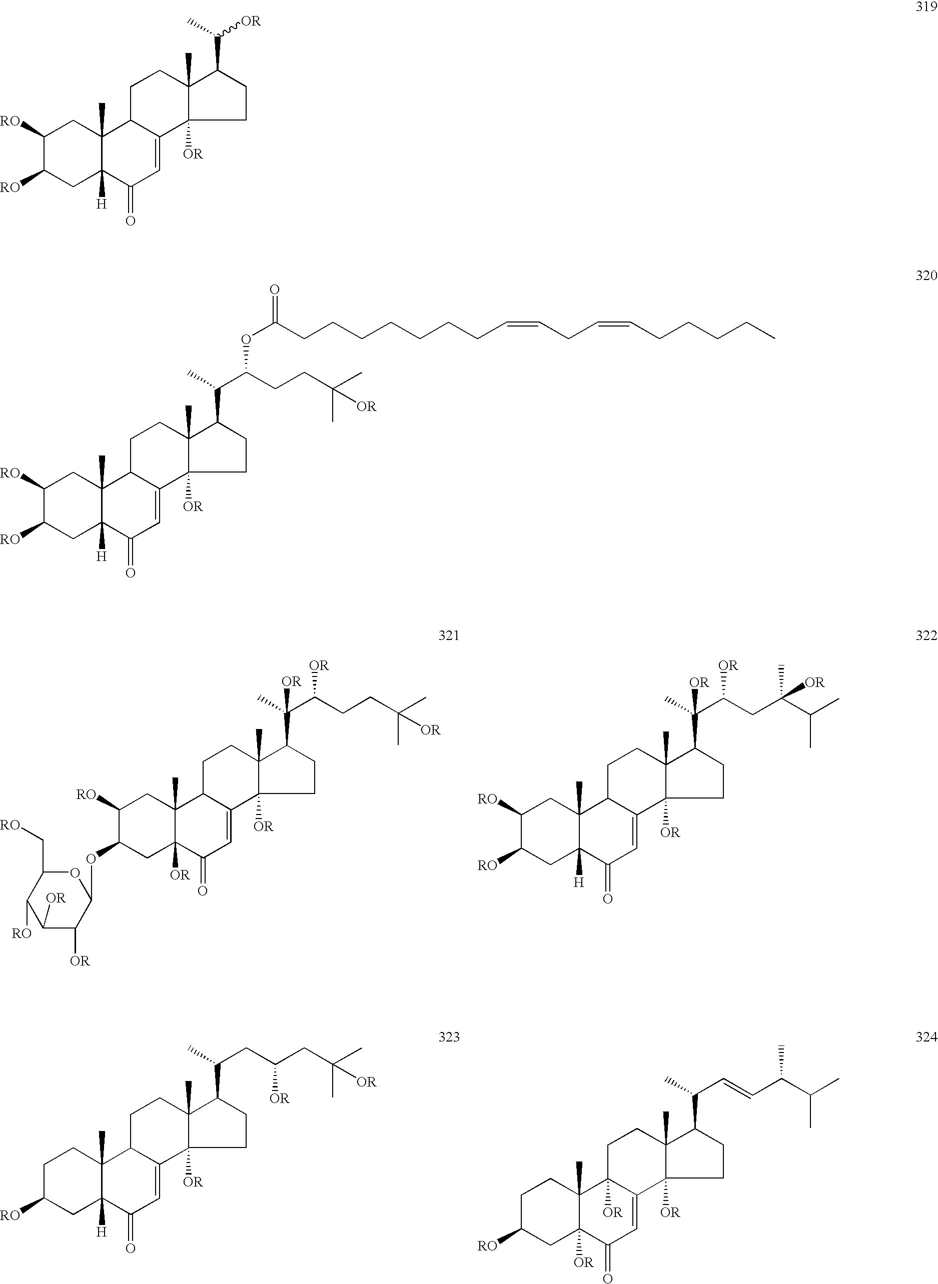 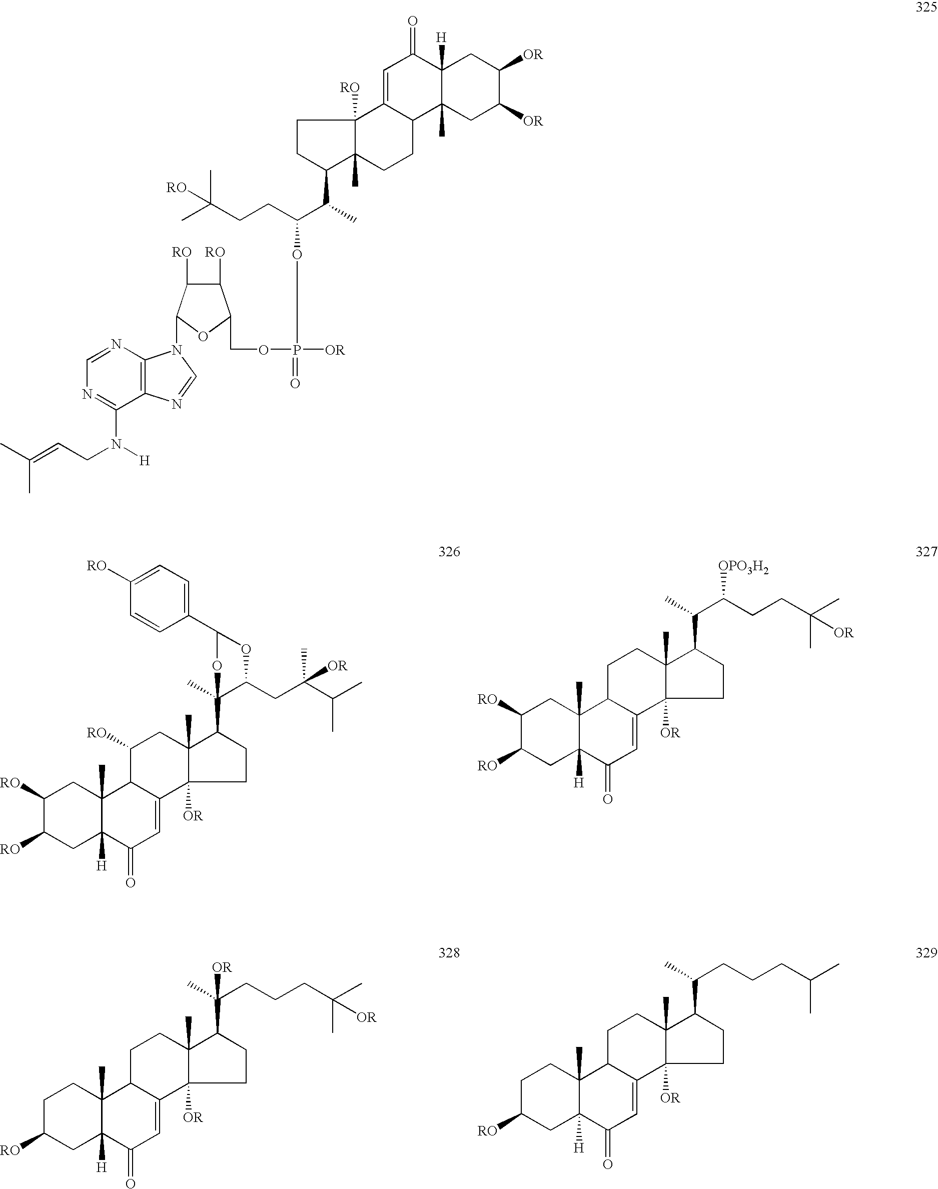 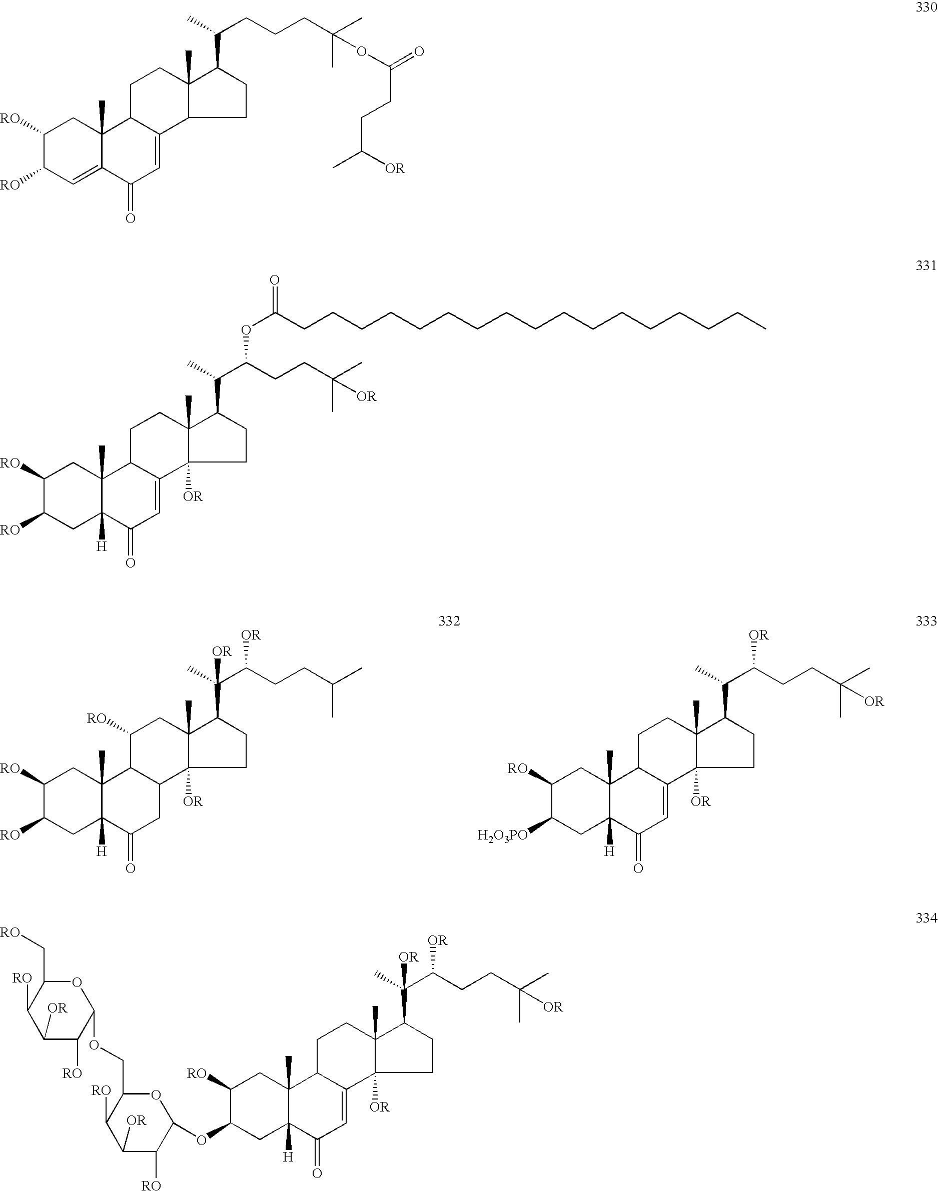 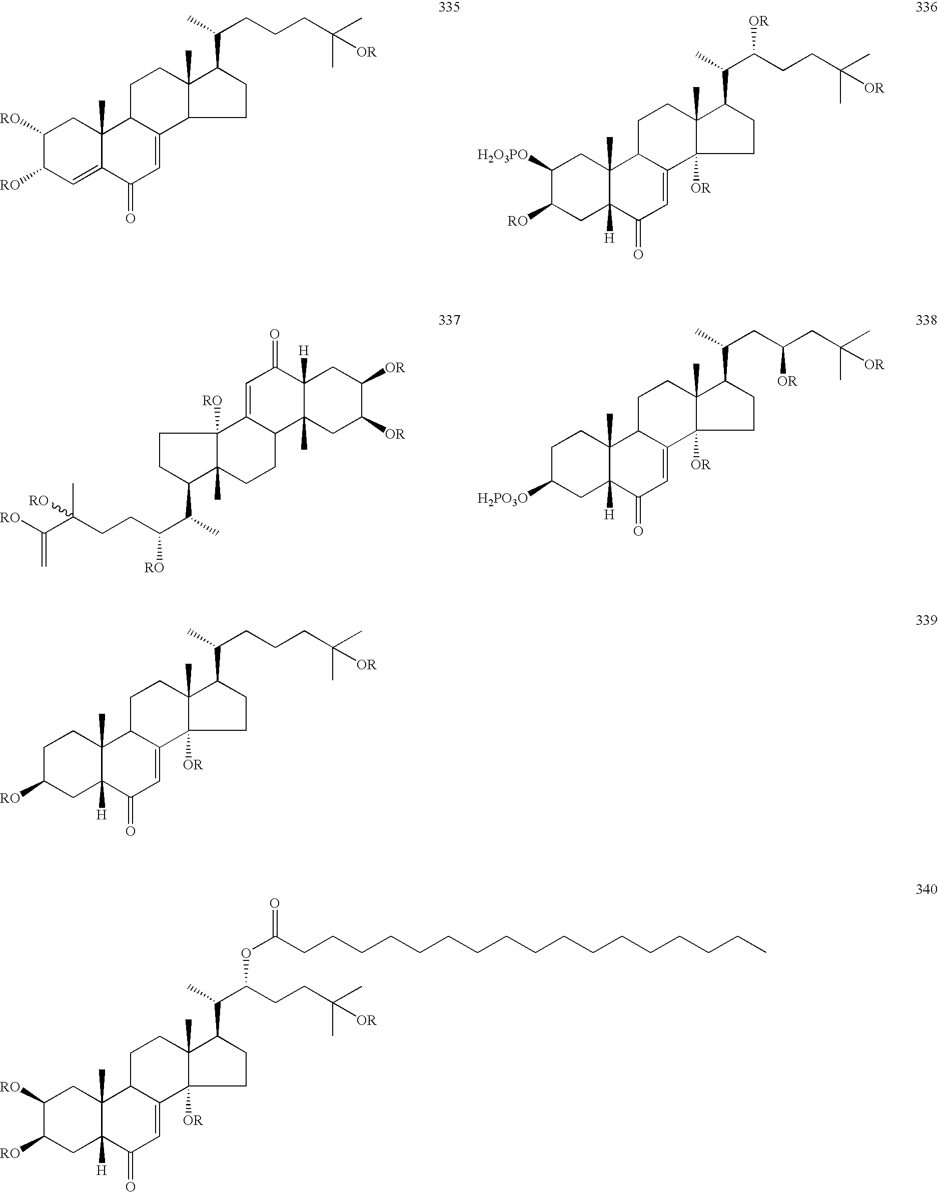 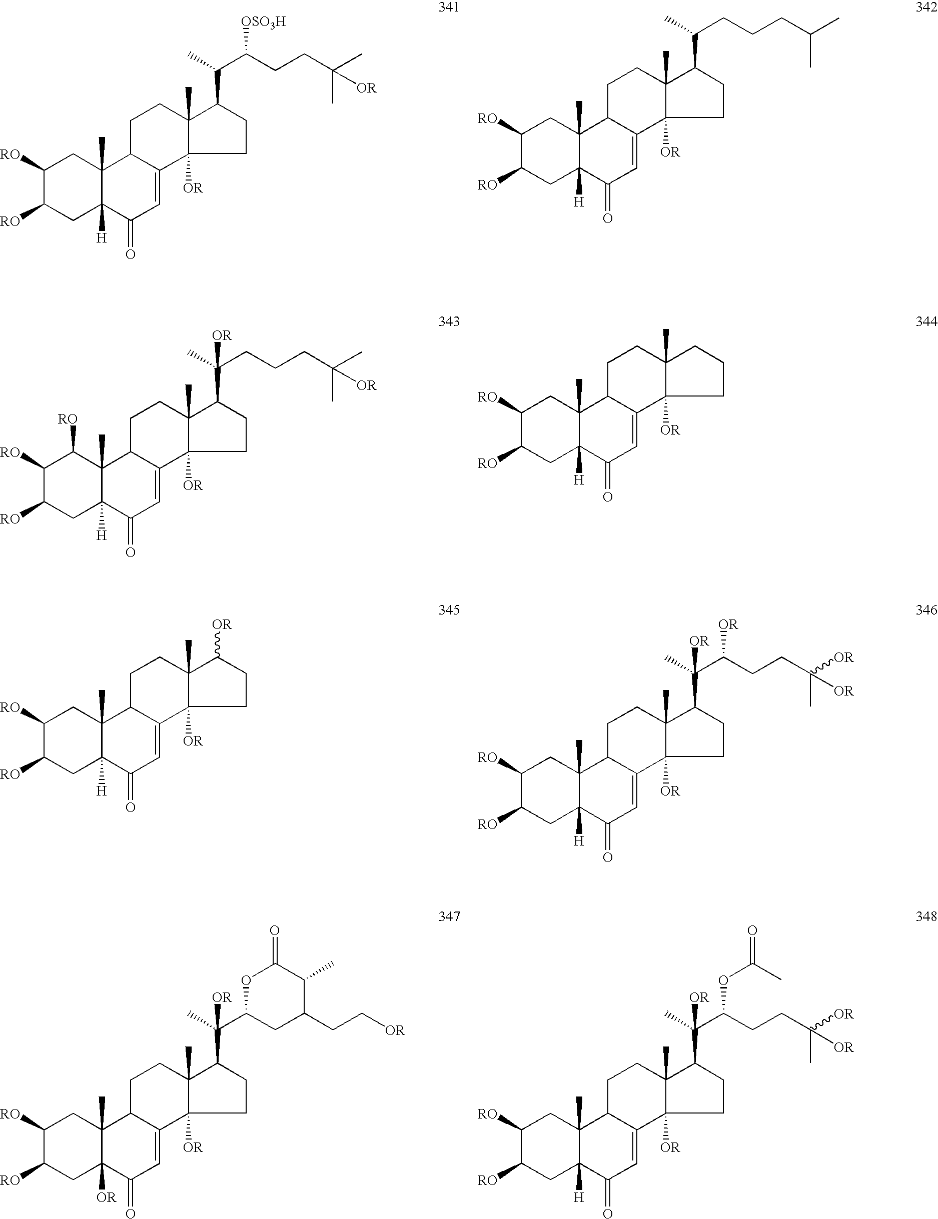 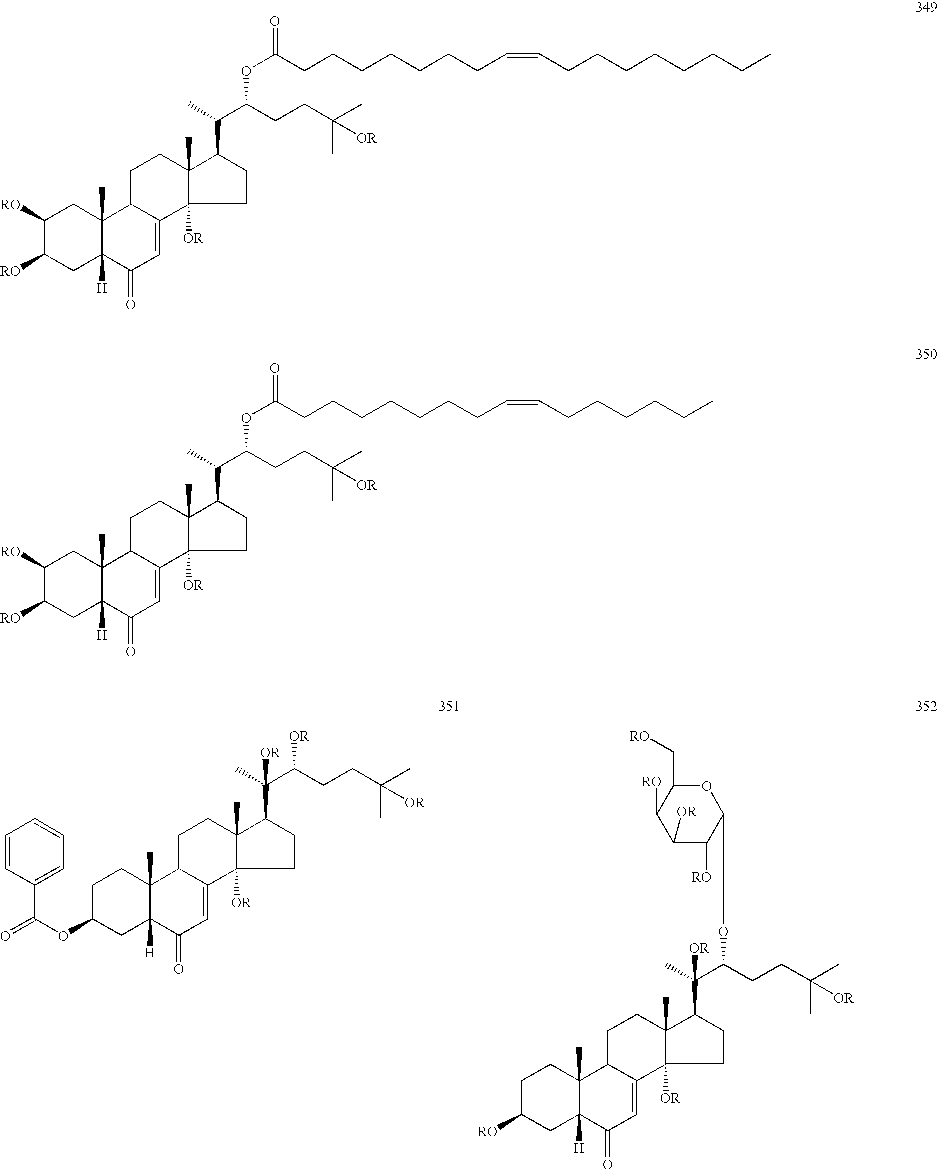 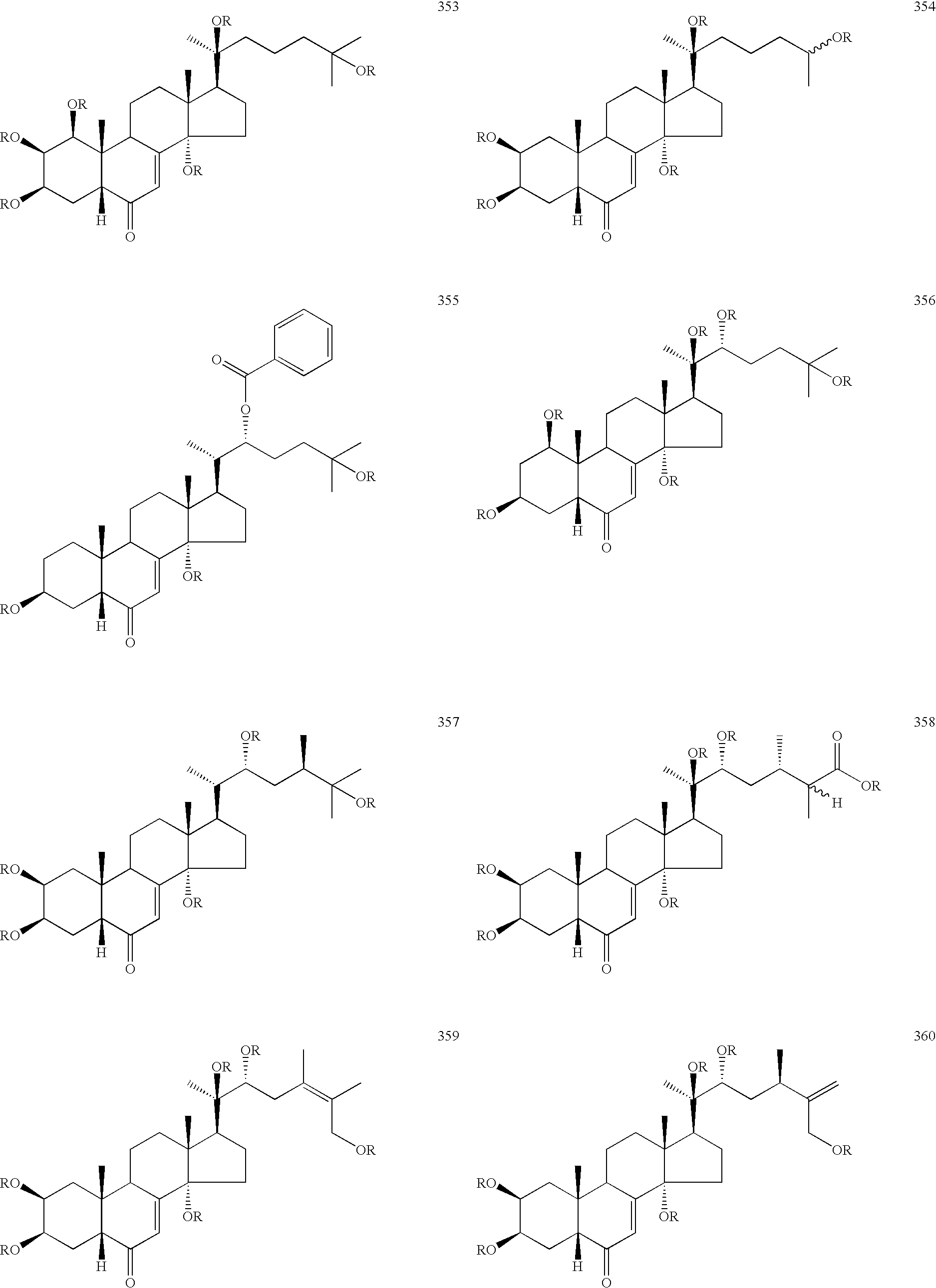 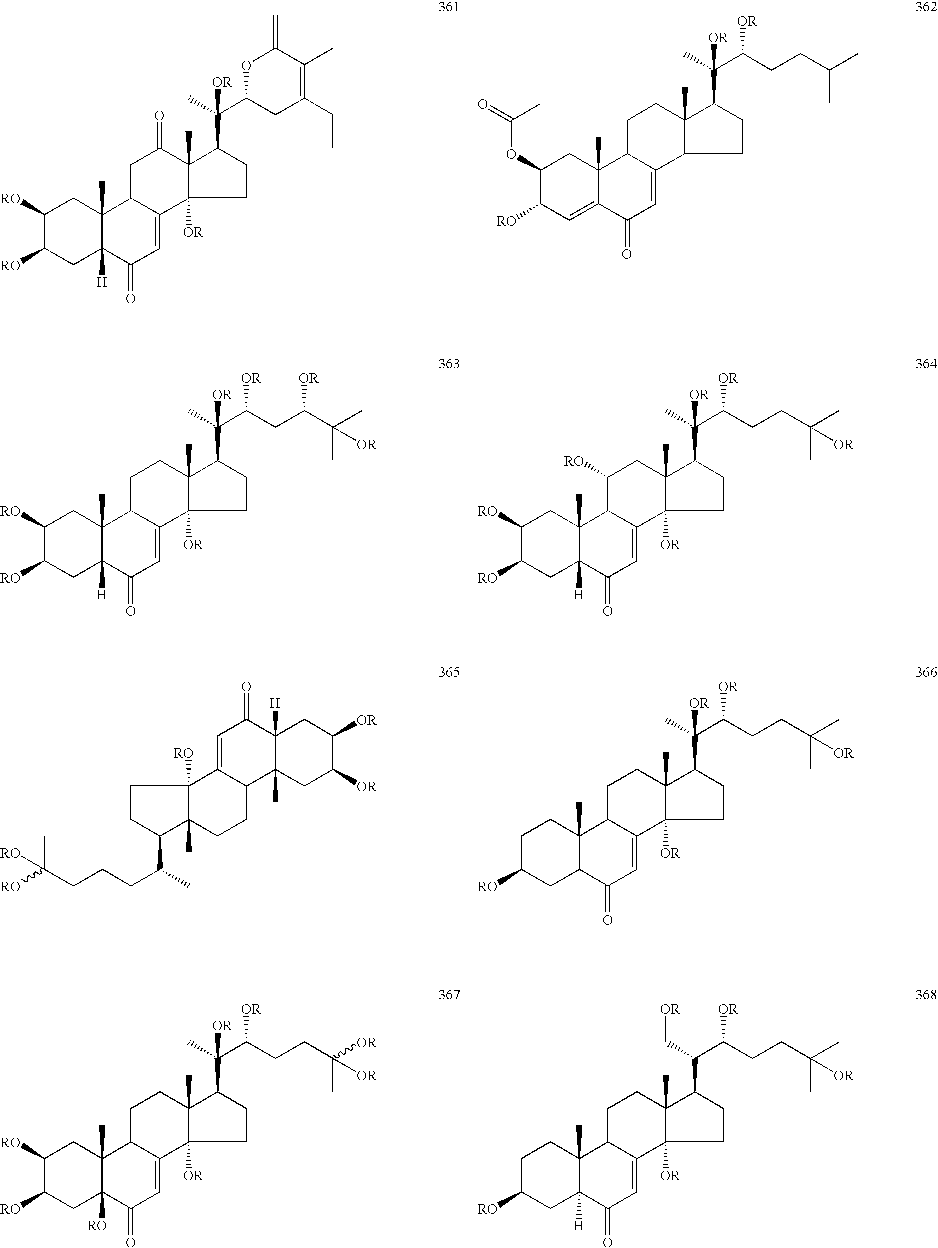 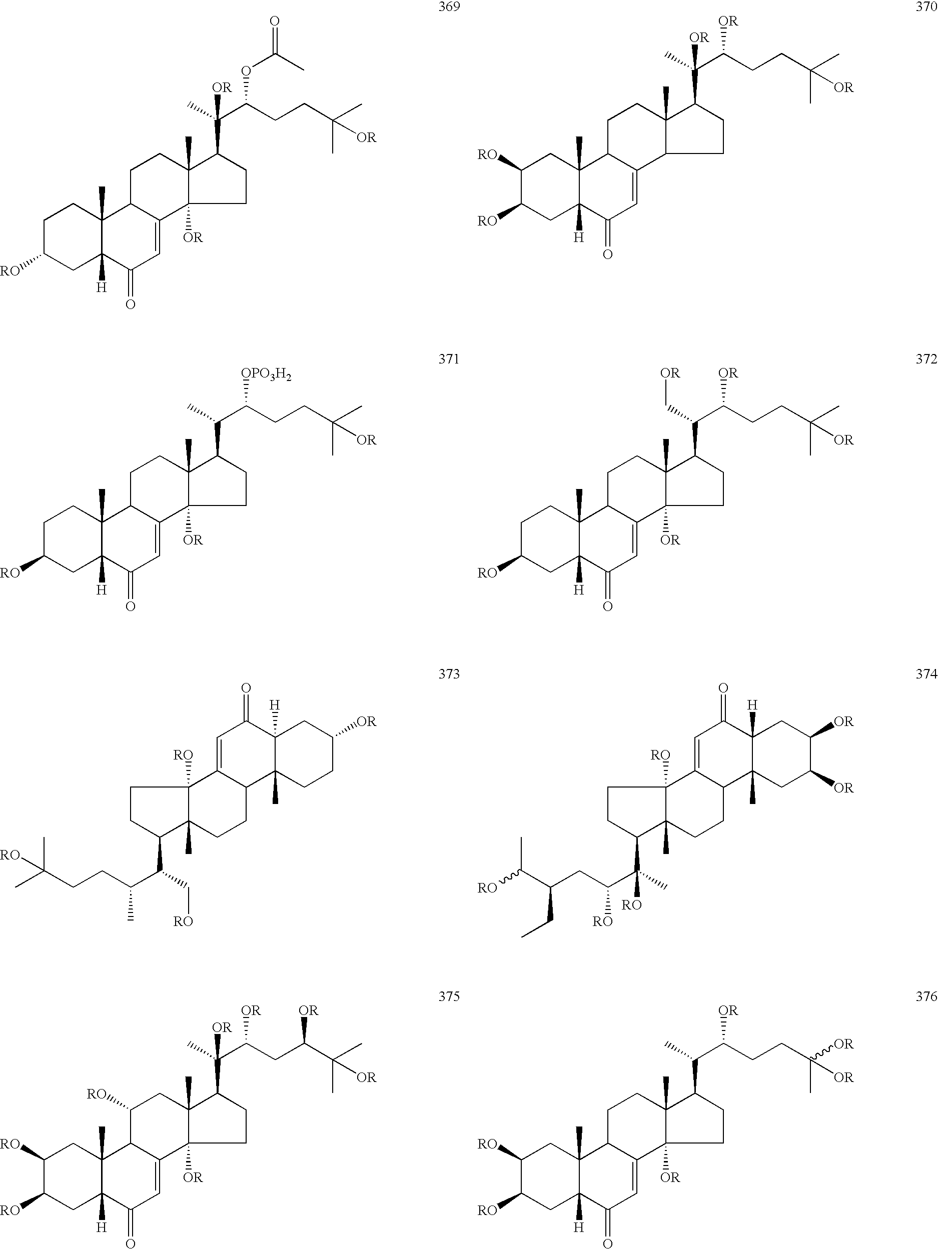 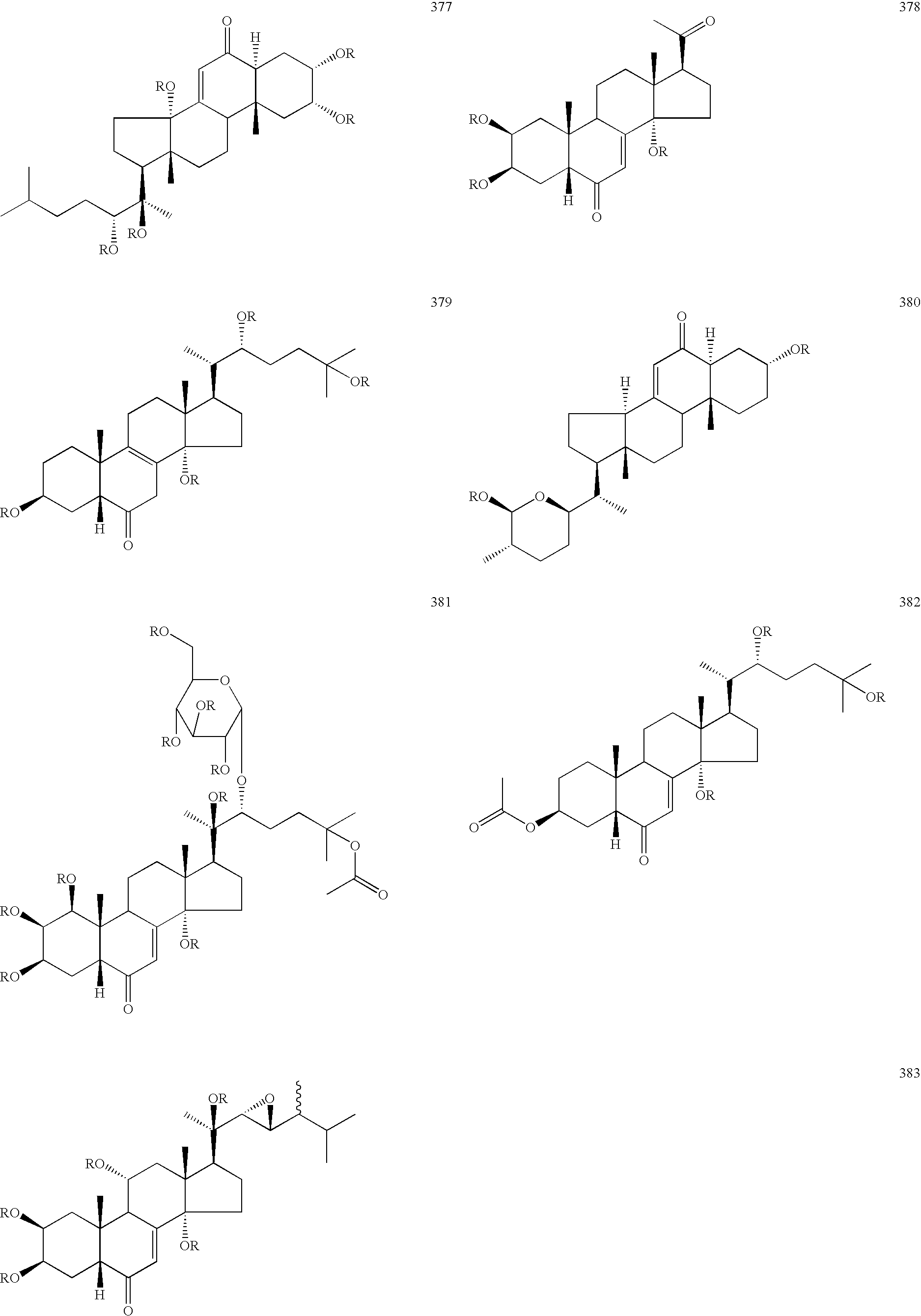
wherein R is a residue of a biologically significant amino acid, or a naturally occurring amino acid, selected from arginine, glycine, alanine, beta-alanine, valine, isovaline, norvaline, leucine, isoleucine, norleucine, alloisoleucine, phenylalanine, proline, serine, homoserine, threonine, allothreonine, tyrosine, cysteine, cystine, methionine, lysine, histidine, homocysteine, tryptophan, aspartic acid, glutamic acid, asparagine, glutamine, taurine, ornithine, citrulliene, sarcosine, aminobutyric acid, aminoisobutyric acid, amino-n-butyric acid, pyroglutamic acid, homoserine, thiaproline, seleno-L-methionine, hydroxyproline, 4-hydroxyproline, 5-hydroxyproline, amino-adipic acid, cadaverine (15-diaminopontane), 4-amino-benzoic acid, x-aminopimelic acid, 2,4-diamino-n-butyric acid, gylcine-glycine, ornithine, glycine-proline, threonine-aspartic acid, hydroxylysine, diaminopimelic acid, proline-hydroxyproline, lysine-alanine, dopamine, cystathionine, 3,4-dihydroxy-phenylalanine, arginine-succinic acid, ethionine, gamma amino butyric acid, statine, carnitine, serotonin, and other naturally occurring amino acids, peptides, oligo-peptides and proteins thereof, and pharmaceutically acceptable salts thereof, and/or
R is one or more of hydrogen, hydroxy, halogen, alkyl, cycloalkyl, alkyl, substituted alkyl, cycloalkyl, substituted cycloalkyl, heterocylclic, substituted heterocyclic, alkenyl, substituted alkenyl, alkynl, substituted alkynyl, aryl, substituted aryl, heteroaryl, substituted hereroaryl, alkylaryl, substituted alkylaryl, arylalkyl, substituted arylalkenyl, substituted arylalkenyl, arylalkynl, substituted arylalkynl, aroyl, substituted aroyl, acyl, substituted acyl, or the like, or the two R groups can cooperate to form a 5-, 6- or 7-membered ring including N and the two R groups, or either of the R groups is a divalent moiety selected from the group consisting of alkylene, substituted alkylene, oxyalklene, substituted oxyalkylene, alkenylene, substituted alkenylene, arylene, substituted arylene, alkarylene, substituted alkarylene, aralkylene and substituted aralkylene, wherein said divalent moiety serves as the same substituent for two dithiocarbamate structures, thereby linking said structures together so as to form a bis(dithiocarbamate) species; tautomers thereof; pharmaceutically acceptable salts thereof; isomers thereof; sterioisomers thereof; enantiomers thereof; diastereoisomers thereof; and racemic mixtures thereof.

Various compounds of this invention may possess asymmetric centers, and may therefore be produced as mixtures of stereoisomers or as individual stereoisomers. Individual stereoisomers may be obtained by using an optically active starting material, by resolving a racemic or non-racemic mixture of an intermediate at an appropriate stage in the synthesis thereof or by resolution of a compound. It is contemplated that the individual stereoisomers and all mixtures of stereoisomers, be they racemic or non-racemic, are included within the scope of the present invention. Compounds of this invention possessing at least one asymmetric center may be produced as mixtures of stereoisomers, or as individual isomers, such as R and S-stereoisomers and L and D-enantiomers. The individual enantiomers may be obtained, for example, by resolving a racemic or non-racemic mixture of some intermediate stage of synthesis.

The term “isomers” refers to different compounds that have the same molecular formula.

The term “stereoisomers” refers to isomers that differ only as to the way the atoms thereof are arranged in space.

The term “enantiomers” refers to a pair of stereoisomers which are not superimposable mirror images of each other.

The term “diastereoisomers” are stereoisomers which are not mirror images of each other.

The term “racemic mixture” refers to a mixture containing equal parts of individual enantiomers.

The term “non-racemic mixture” refers to a mixture containing un-equal parts of individual enantiomers or stereoisomers.

The terms “treatment”, “in need of” and “use” as used herein cover any treatment of a disease and/or a condition and/or the prevention and/or the amelioration to any degree thereof, and/or to enhanced body performance, such increased energy, increased strength, increased muscle mass, increased recovery from fatigue and/or injury, increased dexterity and/or athletic ability, increased stamina, increased memory and intellectual and/or reasoning ability in animals, particularly humans, and is contemplated to include the following:
(i) preventing or inhibiting to any degree a disease and/or condition from occurring or manifesting in a subject, and/or in which said subject may be predisposed to the disease and/or condition, but has not yet been diagnosed as having it;
(ii) inhibiting the disease and/or the condition, or otherwise arresting its development and/or manifestation; or
(iii) relieving the disease and/or condition, or otherwise causing regression of the disease and/or condition; or
(iv) contributing to a feeling of well being of the subject and/or an overall improvement in health of the subject; or
(v) aiding or contributing in any way and to any degree to the enhanced body performance of a subject.

The term “pharmaceutically acceptable salts is an art term which, as known, refers to salts of the subject compounds which possess the desired pharmacological activity, physiological efficacy, or biological activity in general, and which are neither biologically nor otherwise undesirable. These salts can be formed by any known method, such as with inorganic acids, for instance, and without limitation, acetate, adipate, alginate, aspartate, benzoate, benzenesulfonate, bisulfate, butyrate, citrate, camphorate, camphosulfonate, cyclopentanepropionate, digluconate, dodecylsulfonate, ethanesulfonate, fumerate, glucoheptanoate, glycerophosphate, hemisulfate, heptanoate, hexanoate, hydrochloride, dihydrochloride, hydrobromide, hydroiodide, lactate, maleate, methanesulfonate, oxalate, nicotinate, undecanoate, with base salts including ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids as contemplated for use herein; basic nitrogen-containing groups may be quarternized with, for example, with lower alkyl halides, such as methyl, ethyl, propyl and butyl chloride, bromides and iodides and the like; also contemplated are dialkyl sulfates, such as dimethyl, diethyl, dibutyl and diamyl sulfates, long chain halides, such as decyl, lauryl, myristyl and stearyl chlorides, bromides, and iodides, alkyl halides, such as benzyl and phenethyl bromides and the like, to obtain water or oil-soluble and/or dispersible products as desired.

The term “biologically significant amino acid” refers to any amino acid known in the art to be bio-active in mammals to any degree, or which displays any amount of efficacy for any indication in humans and animals. See, for example, Chemical Abstracts, Naming and Indexing of Chemical Substances (1987).

“Oligo-peptides” as used herein refer to small chain amino acid combinations, such as, for instance, synthetic growth hormone releasing peptides (GHRPs), of which an example is ghrelin, a 28 amino acid octanoylated peptide that is produced by the stomach and which plays an important role in tissue growth and recovery from injury.

The term “alkyl” as used herein refers to straight chain, branched and cyclic fully saturated hydrocarbon residues, preferably straight chain or branched alkyl. Some non-limiting examples useful herein of straight and branched alkyl moieties of the invention include C1-20 alkyl, such as methyl, ethyl, propyl, isopropyl, butyl, isobutyl, see-butyl, tert-butyl, amyl, isoamyl, sec-amyl, 1,2-dimethylpropyl, 1,1-dimethylpropyl, hexyl, 4-methylpentyl, 1 methylpentyl, 2-methylpentyl, 3-methylpentyl, 1,1-demethylbutyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, heptyl, 5-methylhexyl, 1,methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,2,2-trimethylbutyl, 1,1,3-trimethylbutyl, octyl, 6-methylheptyl, 1 methylheptyl, 1,1,3,3-tetramethylbutyl, nonyl, 1, 2, 3, 4, 5, 6, or 7-methyloctyl, 1, 2, 3, 4, or 5-ethylheptyl, 1, 2, or 3-propylhexyl, decyl, 1, 2, 3, 4, 5, 6, 7 and 8-methylnonyl, 1, 2, 3, 4, 5, or 6-ethyloctyl, 1, 2, 3, or 4-propyllheptyl, undecyl, 1, 2, 3, 4, 5, 6, 7, 8 or 9-methyldecyl, 1, 2, 3, 4, 5, 6, or 7-ethylnonyl, 1, 2, 3, 4, or 5-propyloctyl, 1, 2, or 3-butylheptyl, 1-pentylhexyl, dodecyl, 1, 2, 3, 4, 5, 6, 7, 8, 9, or 10-methylundecyl, 1, 2, 3, 4, 5, 6, 7, or 8-ethyldecyl, 1, 2, 3, 4, 5, or 6-propylnonyl, 1, 2, 3, or 4-butyloctyl or 1,2-pentylheptyl and the like.

Additional alkyl examples include C 21-25 alkyl, C 26-30 alkyl, C 31-35 alkyl, C 36-40 alkyl, C 41-46 alkyl, C 47-55 alkyl, C 56-60 alkyl, with examples of cyclic alkyls including mono- and polycyclic alkyl groups, such as cyclopropyl, cyclobutyl, cyclohexyl, cycloheptyl, cyclooctyl, cyclononyl, cyclodecyl and the like.

Further, as used herein, the term “alkenyl” refers to groups formed from straight chain, branched or cyclic hydrocarbon residues containing at least one carbon-carbon double bond, including ethylenically mono-, di-, or polyunsaturated alkyl or cycloalkyl groups as exemplified and defined above. Some representative examples of alkenyl groups or moieties include C1-20 alkenyl, such as vinyl, allyl, 1-methylvinyl, butenyl, iso-butenyl, 3-methyl-2-butenyl, 1-pentenyl, cyclopentyl, 1-methylcyclopentyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1,4-pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cycloheptatrienyl, and 1,3,5,7-cyclooctatetraenyl.

Additional examples of alkenyl groups include C 10-15 alkenyl, C 16-20 alkenyl, C 21-25 alkenyl, C 26-30 alkenyl, C 31-35 alkenyl, C 36-40 alkenyl, C 41-45 alkenyl, C 46-50 alkenyl, C 51-55 alkenyl, C 56-60 alkenyl, C 61-65 alkenyl, C 66-70 alkenyl, and C 71-80 alkenyl, with each of these examples possibly containing one or more alkyl or alkyl branches.

As employed herein, “substituted alkyl” comprises alkyl groups further bearing one or more substituents selected from hydroxyl, alkoxy (of a lower alkyl group), mercapto (of a lower alkyl group), cycloalkyl, substituted cycloalkyl, heteroaryl, aryloxy, substituted aryloxy, halogen, trifluoromethyl, cyano, nitro, nitrone, amino, amido, —C(O)H, acyl, oxyacyl, carboxyl, carbamate, sulfonyl, sulfonamide, sulfuryl, and the like.

As employed herein, “cycloalkyl” refers to cyclic ring-containing groups containing in the range of about 3 up to 8 carbon atoms, and “substituted cycloalkyl” refers to cycloalkyl groups further bearing one or more substituents as set forth above.

As employed herein, “heterocyclic” refers to cyclic (i.e., ring-containing) groups containing one or more heteroatoms (e.g., N, O, S, or the like) as part of the ring structure, and having in the range of 3 up to 14 carbon atoms and “substituted heterocyclic” refers to heterocyclic groups further bearing one or more substituents as set forth above.

As employed herein, “alkenyl” refers to straight or branched chain hydrocarbyl groups having a least one carbon-carbon double bond, and having in the range of about 2 up to 12 carbon atoms, and “substituted alkenyl” refers to alkenyl groups further bearing one or more substituents as set forth above.

As employed herein, “alkynyl” refers to straight or branched chain hydrocarbyl groups having at least one carbon-carbon triple bond, and having in the range of about 2 up to 12 carbon atoms, and “substituted alkynyl” refers to alkynyl groups further bearing one or more substituents as set forth above.

As employed herein, “aryl” refers to aromatic groups having in the range of 6 up to 14 carbon atoms and “substituted aryl” refers to aryl groups further bearing one or more substituents as set forth above.

As employed herein, “heteroaryl” refers to aromatic groups containing one or more heteroatoms (e.g., N, O, S, or the like) as part of the ring structure, and having in the range of 3 up to 14 carbon atoms and “substituted heteroaryl” refers to heteroaryl groups further bearing one or more substituents as set forth above.

As employed herein, “alkylaryl” refers to alkyl-substituted aryl groups and “substituted alkylaryl” refers to alkylaryl groups further bearing one or more substituents as set forth above.

As employed herein, arylalkyl” refers to aryl-substituted alkyl groups and “substituted arylalkyl” refers to arylalkyl groups further bearing one or more substituents as set forth above.

As employed herein, “arylalkenyl” refers to aryl-substituted alkenyl groups and “substituted arylalkenyl” refers to arylalkenyl groups further bearing one or more substituents as set forth above.

As employed herein, “arylalkynyll” refers to aryl-substituted alkynyl groups and “substituted arylalkynyl” refers to arylalkynyl groups further bearing one or more substituents as set forth above.

As employed herein, “aroyl” refers to aryl-carbonyl species such as benzoyl and “substituted aroyl” refers to aroyl groups further bearing one or more substituents as set forth above.

As employed herein, “acyl” refers to alkyl-carbonyl species.

As employed herein, “halogen” refers to fluoride, chloride, bromide or iodide atoms.

Some non-limiting examples of the ester compounds which may be used herein include methyl formate, propyl formate, isobutyl formate, butyl formate, methyl acetate, ethyl acetate, propyl acetate, isopropyl acetate, sec-butyl acetate, isobutyl acetate, butyl acetate, amyl acetate, sec-amyl acetate, iso-amyl acetate, sec-hexyl acetate, hexyl acetate, heptyl acetate, octyl acetate, methyl propionate, methyl butyrate, isobutyl isobutyrate, butyl butyrate, methyl methanoate, ethyl methanoate, butyl methanoate, methyl ethanoate, ethyl ethanoate, propyl ethanoate, 2-propyl ethanoate, butyl ethanoate, dimethyl ethyl ethanoate, methylpropyl ethanoate, pentyl ethanoate, 2 pentyl ethanoate, 3-methylbutyl ethanoate, hexyl ethanoate, 2-heptyl ethanoate, octyl ethanoate, 2-ethylhexyl ethanoate, nonyl ethanoate, methyl propanoate, ethyl propanoate, propyl propanoate, butyl propanoate, dimethyl ethyl propanoate, 3-methylbutyl propanoate, hexyl propanoate, heptyl propanoate, alkyl undecanoate, carbonate and polycarbonate esters.

Some particularly preferred, but non-limiting, examples include, for instance, testosterone undecanoate, other steroidal tetracyclic undecanoate esters, such as any of the above-described compounds, and various ester derivatives of phytoecdysteroids, such as esters of 20-hydroxyecdysone or 20-beta-hydroxy-ecdysone (7,(5b) cholesten-2b,3b,14a,20R,22R,25-hexol-6-one) and 22-dehydro-20-hydroxyecdysone, and more particularly 20-beta-hydroxyecdysone-2-acetate, 20-beta-hydroxyecdysone-triacetate, and 20-beta-hydroxyecdysone-tetraacetate.

### Preparation

The preparation of the ester derivatives of the invention may be accomplished by any conventional method, of which several are known. For example, U.S. Pat. No. 3,872,099 describe methods for producing active amino acid esters. See also U.S. patent application 20040030177. Other examples are described in U.S. Pat. No. 5,113,009 for the preparation and isolation of a mineral acid salt of an amino acid methyl ester. In this example, a mineral acid is employed to form a salt with the amino group of the amino acid to function as a catalyst for esterification of the carboxylic acid group of the amino acid with an alkyl alcohol, such as methanol. Still other examples of amino acid ester synthesis may be found in EP 1044 676 and EPO 928 608 by Ajinomoto Co., which is also an excellent source for obtaining pharmaceutical grade amino acids, preferably in crystalline form for use in the present invention.

Still other examples of ester synthesis may be found in Daniel, et al., Tetrahedron, Vol. 53, No. 16, pp. 5855-5822 (1997) which reports the regioselective esterification of ecdysteroids at the C-2OH, as catalyzed by Candida antarctica lipase B, and U.S. Pat. No. 2,781,368 discussing the preparation of testosterone esters.

In general, tetracyclic polyhydric, or polyhydroxylated compounds having a generic steroidal-like structure are contemplated for use as substrates in the production of the inventive esterified compounds, such as phytoecdysteroids, for use in the formulations and preparations of the invention. Some non-limiting examples include, cyasterone, turkesterone, turkesterone mono-, di-, and tri-acetates, viticosterone E, 2-acetate hydroxyecdysone, 3-hydroxyecdysone, 2-dehydro-3-epi-20-hydroxyecdysone, 22-dehydro-12-hydroxyecdysone, 3-dehydro-20-hydroxyecdysone, 4-dehydro-20-hydroxyecdysone, 24(241)-dehydro-242-hydroxy-makisterone c, 22-dehydro-12-hydroxy-29-nor-cyasterone, 22-dehydro-12-hydroxy-29-nor-sengosterone, 22-dehydro-12-hydroxy-sengosterone, 24(28)-dehydromakisterone A, 24,25-dehydroprecyasterone, 3-deoxy-1,20-hydroxyecdysone, 2-deoxycasterone, 22-deoxy-20,21-dihydroxyecdysone, 22-deoxy-20,26-dihydroxyecdysone, 14-deoxyecdysone, 22-deoxyecdysone, 25-deoxyecdysone, 2-deoxyecdysone, 2-deoxyecdysone 22-acetate, 2-deoxyecdysone 3-acetate, 2-deoxyecdysone 22-adenosine-monophosphate, 2-deoxyecdysone 22-benzoate, 2-deoxyecdysone 22-phosphate, 14-deoxy-hydroxyecdysone, 22-deoxy-26-hydroxyecdysone, 2-deoxy-20-hydroxyecdysone, 2-deoxy-21-hydroxyecdysone, 5a-2-deoxy-21-hydroxyecdysone, 2-deoxy-20-hydroxyecdysone 22-acetate, 2-deoxy-20-hydroxyecdysone 3-acetate, 2-deoxy-20-hydroxyecdysone 3-acetate, 5a-2-deoxy-20-hydroxyecdysone 3-acetate, 2-hydroxy-20-hydroxyecdysone 22-benzoate, 2-deoxy-20-hydroxyecdysone 3-crotonate, 2-deoxy-20-hydroxyecdysone 3,22-diacetate, 2-deoxy-20-hydroxyecdysone 3-glucoside, 2-deoxy-20-hydroxyecdysone 20,22-monoacetonide, 22-deoxy-20-hydroxyecdysone 3-phosphate, 2-deoxy-20-hydroxyecdysone 22-phosphate, 2-deoxy-20-hydroxyecdysone-25-acetate, 2-deoxy-20-hydroxyecdysone-22-glucoside, 2-deoxy-20-hydroxyecdysone-3-o-benzoate, 25-deoxy-20-hydroxyecdysonoic acid, 22-deoxyinokosterone, 22-deoxy-integristerone A, abutasterone, 3-acetyl-20-hydroxyecdysone 2-phosphate, 3-acetyl-14a-hydroxypinnasterol, 2-acetyl-29-norcyasterone, 3-acetyl-29-norcyasterone, acetylpinnasterol, ajugalactone, ajugasterone B, ajugasterone B′, ajugasterone C, ajugasterone C 20,22-mono-acetonide, ajugasterone D, amarasterone A, amarasterone B, asterasterol B, atrotosterone A, atrotosterone B, atrotosterone C, blechnoside A, syn. Silenoside E, blechnoside B, bombycosterol, brahuisterone, calonysterone, calvasterol A, calvasterol B, canescensterone, capitasterone, carpesterol, carthamosterone, carthamosterone A, carthamosterone B, castasterone, cheilanthone A, cheilanthone B, coronatasterone, cyanosterone A, cyasterone 22-acetate, cyasterone 3-acetate, cyathisterone, dacryhainansterone, syn. 5-deoxykaladasterone, dacrysterone, decumbesterone A, 2-dehydroajugalctone, 3-dehydroajugalactone, 24(241)[Z]-dehydroamarasterone B, 3-dehydroecdysone, rubrosterone, rhapontisterone (punisterone), la, 20R-dihydroxyecdysone, rapisterone D 20-acetate, rapisterone D, rapisterone C, rapisterone B, rapisterone, pterosterone 24-o-Beta-D-glucoside, pterosterone, precyasterone, 3B,5a,9a-trihydroxy-ergosta-7,22-dien-6-one, 3B,5a,14a-trihydroxy-ergosta-7,22-dien-6-one, venustone, viticosterone E 22-benzoate, viticosterone E, vitexirone, viperidone, zoanthusterone, 11a,20,26-trihydroxyecdysone, (24R)-11a,20,24-trihydroxyecdysone, 2,22,25-trideoxyecdysone, tomentesterone B, tomentestrone A, 2,14,22,25-tetradeoxyecdysone, tenuifolioside B, tenuifolioside A, taxisterone, stachysterone C, stachysterone B, stachysterone A, sogdisteronesilenosterone, syn. 3-dehydro-2-deoxyecdysone, 5a-silenoside E, sileneoside H, sileneoside G, sileneoside F, sileneoside D, sileneoside C, sileneoside B, sileneoside A, sidisterone, sidasterone B, sidasterone A, shidasterone (stachysterone D), sengosterone, schizaeasterone B, schizaeasterone A, scabrasterone, praemixisterone, poststerone, ponasteroside A, ponasterone C₂-cinnamate, ponasterone C, ponasterone B, ponasterone A 22-glycolate, ponasterone A, polyporusterone G, polyporusterone F, polyporusterone E, polyporusterone D, polyporusterone C, polyporusterone B, polyporusterone A, polypodoside C, polypodoside B, polypodoside A, polypodosaponin, polypodogenin, polypodoaurien, nusilsterone, 29-norsengosterone, 29-norcyasterone, muristerone A, melandrioside A, malacosterone, makisterone D, makisterone C, syn. Podecdysone A, lemmasterone, makisterone B, makisterone A, limnantheoside B, limnantheoside A, leuzeasterone, lesterone, kaladasterone, isovitexirone, isocyasterone, integristerone B, integristerone A 25-acetate, integristerone A, inokosterone, syn. Callinecdysone A, 5-Beta-hydroxrubrosterone, 26-hydroxy-polypodine B, 26-hydroxypinnatasterone, 14a-hydroxypinnasterol, 25-hydroxypanuosterone, 20-hydroxy-24-hydroxymethylecdysone, 20-hydroxyecdysonoic acid, 5a-20-hydroxyecdysone, syn. Epiecdysterone, 20-hydroxyecdysone 22-acetate, 26-hydroxyecdysone 2-phosphate, 26-hydroxyecdysone 26-phosphate, 20-hydroxyecdysone 22-phosphate, 20-hydroxyecdysone 3-p-courmatate, 20-hydroxyecdysone 22-palmitate, 20-hydroxyecdysone 22-oleate, 20-hydroxyecdysone 20,22-mono-acetonide, 20-hydroxyecdysone 2,3-monoacetonide, 20-hydroxyecdysone 22-linoleate, 20-hydroxyecdysone 22-glycolate, 26-hydroxyecdysone 22-glucoside, 20-hydroxyecdysone 22,25-dibenzoate, 20-hydroxyecdysone 2,3;20,22-diacetonide, 20-hydroxyecdysone 2,22-diacetate, 20-hydroxyecdysone 3,22-diacetate, 20-hydroxyecdysone 3-Beta-D-Glucoside, 20-hydroxyecdysone 25-beta-D-glucoside, 20-hydroxyecdysone 2-beta-D-gluco-pyranoside, 20-hydroxyecdysone 2-cinnamate, 20-hydroxyecdysone 2-cinnamate, 20-hydroxyecdysone 22-benzoate 25-glucoside, 5a-20-hydroxyecdysone 22-benzoate, 20-hydroxyecdysone 22-benzoate, 20-hydroxyecdysone 20-benzoate, 20-hydroxyecdysone, 20-hydroxyecdysone 3-acetate, 20-hydroxyecdysone 2-acetate, 20-hydroxyecdysone 22-acetate, 20-hydroxyecdysone 3(/2)-phosphate, 20-hydroxyecdysone 3-(/2)acetate 22-phosphate, 26-hydroxyecdysone, 24-hydroxyecdysone, 20-hydroxyecdysone, 11a-hydroxyecdysone, 24-hydroxy-dihydrocarthamosterone, 24-hydroxy-cyasterone, 14a-hydroxycarpesterol, palythoalone A, 22-oxo-20-hydroxyecdysone, 22-oxo-cyasterone, osladin, nusilsterone, 5a-polypodine B 3-beta-D-glucoside, polypodine B 2-cinnamate, polypodine B 22-benzoate, polypodine B 20,22-acetonide, polypodine B 22-acetate, polypodine B, 5a-polypodine B, podecysone B 25-o-beta-D-glucoside, podecdysone B, pinnatasterone, pinnasterol, 2-beta, 3-beta, 6a,22R,25-pentahydroxy-5-beta-cholestane, 2-beta, 3a,6a,22R,25-pentahydroxy-5-beta-cholestane, paxillosterone 20,22-p-hydroxy-benzylidene acetyl, paxillosterone 20,22-p-hydroxy-benzylidene acetyl, paxillosterone, paristerone, panuosterone, palythoalone B, polypodine B 3-beta-D-glucoside, 3-epi-20-hydroxyecdysone 3-phosphate, 25-hydroxyatrotosterone B, 25-hydroxyatrotosterone A, 14a-hydroxy-acetylpinnasterol, 5-hydroxyabutasterone, 3-beta, 14a,17a,20,24,25-hexahydroxy-5a-ergosta-7,22-dien-6-one, gymnasterone B, gerardiasterone, 3-beta, 5a, 9a,14a-(22 E,24R)-tetrahydroxy-ergosta-7,22-D, ergosta-4,7,22-triene-3,6-dione, 28-epi-sengosterone, 3-epi-rubrosterone, 24-epi-pterosterone, 3-epi-poststerone, 24-epi-pinnatasterone, 24-epi-makisterone A, 3-epi-26-hydroxyecdysone, 3-epi-20-hydroxyecdysone, 22-epi-20-hydroxyecdysone, 22-epi-14a-hydroxy-acetylpinnasterol, 3-epi-ecdysone 22-phosphate, 3-epi-ecdysone, 3-epi-20,26-dihydroxyecdysone, 3-epi-22-deoxy-16-beta, 20-dihydroxy-ecdysone-2-phosphate, 3-epi-22-deoxy-16-beta, 20-dihydroxy-ecdysone, 3-epi-22-deoxy-20-hydroxyecdysone-2-phosphate, 3-epi-2-deoxy-20-hydroxyecdysone, 3-epi-22-deoxy-20-hydroxyecdysone, 3-epi-2-deoxyecdysone 3-phosphate, 3-epi-2-deoxyecdysone 22-phosphate, 3-epi-2-deoxyecdysone, 3-epi-22-deoxy-20,26-dihydroxy-ecdysone-2-phosphate, 3-epi-22-deoxy-20,26-dihydroxy-ecdysone, 3-epi-22-deoxy-20,26-dihydroxy-ecdysone, 3-epi-cyasterone 22-acetate, epicyasterone, 3-epi-cyasterone, 24-epi-abutasterone, ecdysteroside, ecdysonoic acid, ecdysone 22-sulfate, ecdysone 22-stearate, ecdysone 3-phosphate, ecdysone 2-phosphate, ecdysone 22-phosphate, ecdysone 22-palmitoleate, ecdysone 22-palmitate, ecdysone 22-oleate, ecdysone, ecdysone 25-o-beta-D-glucopyranoside, ecdysone 22-N-6-(isopentenyl)-adenosine-monophosphate, ecdysone 22-linoleate, ecdysone 22-glycolate, ecdysone 22-glucoside, ecdysone 2,3-diacetate 22-phosphate, ecdysone 22-adenosinemonophosphate, ecdysone 3-acetate 2-phosphate, ecdysone 3-acetate, ecdysone 3(/2)acetate 22-phosphate, ecdysone, 20,26-dihydroxyecdysone 22-acetate, 20,26-dihydroxyecdysone (podecdysone), 2,29-dihydroxycapitasterone, dihydrorubrosterone, 5a-dihydrorubrosterone, dihydropoststerone, 7,8-dihydroajugasterone, 2,22-dideoxy-23-hydroxyecdysone-3-phosphate, 2,22-dideoxy-20-hydroxyecdysone 3-phosphate, 2,22-dideoxy-23-hydroxyecdysone, 2,22-dideoxy-20-hydroxyecdysone, 22,25-dideoxyecdysone, 2,22-dideoxyecdysone, diaulusterol B, diaulusterol A, deoxyviperidone, syn. 5a-ketol, 20-deoxymakisterone A (24-methyl-ecdysone), 5a-22-deoxy-integristerone A, 2-deoxyintegristerone A, 22-deoxy-integristerone A, 22-deoxyinokosterone, 25-deoxy-20-hydroxyecdysonoic acid, 2-deoxy-20-hydroxyecdysone-3-o-benzoate, 2-deoxy-20-hydroxyecdysone-22-glucoside, 2-deoxy-20-hydroxyecdysone-25-acetate, 22-deoxy-20-hydroxyecdysone 3-phosphate, 2-deoxy-20-hydroxyecdysone 20,22-monoacetonide, 2-deoxy-20-hydroxyecdysone 3-glucoside, 2-deoxy-20-hydroxyecdysone 3,22-diacetate, 2-deoxy-20-hydroxyecdysone 3-crotonate, 2-deoxy-20-hydroxyecdysone 22-benzoate, 2-deoxy-20-hydroxyecdysone 22-benzoate, 5a-2-deoxy-20-hydroxyecdysone 3-acetate, 2-deoxy-20-hydroxyecdysone 3-acetate, 2-deoxy-20-hydroxyecdysone 22-acetate, 5a-2-deoxy-21-hydroxyecdysone, 2-deoxy-21-hydroxyecdysone, 2-deoxy-20-hydroxyecdysone, 22-deoxy-26-hydroxyecdysone, 14-deoxy-20-hydroxyecdysone, 2-deoxyecdysone 22-phosphate, 2-deoxyecdysone 22-benzoate, 2-deoxyecdysone 22-adenosine monophpsphate, 2-deoxyecdysone 3-acetate, 2-deoxyecdysone 22-acetate, 2-deoxyecdysone, 25-deoxyecdysone, 22-deoxyecdysone, 14-deoxyecdysone, 22-deoxy-20,26-dihydroxyecdysone, 22-deoxy-20,21-dihydroxyecdysone, 2-deoxycastasterone, 3-deoxy-1,20-dihydroxyecdysone, 20-beta-hydroxyecdysone, vitokosterone, turkesterone 2-acetate, turkesterone 22-acetate, turkesterone 2,11-diacetate, turkesterone 11,22-diacetate, 2,3-isopropylidene turkesteronemuristerone and muristerone A.

### EXAMPLES

Reaction 1 The regioselective synthesis of 20-hyroxyecdysone 2-acetate was undertaken using reaction conditions described in Tetrahedron 1997, 53, 5855-5862. Thus 20B-hydroxyecdysone (20BECD, 0.1025 g, 0.213 mmole) was weighed into a 20 ml scintillation vial, and addition of t-amyl alcohol (10 ml), followed by pyridine (1 ml) to give a clear solution. Vinyl acetate (200 uL, 2.17 mmole) was added to the vial, followed by lipase (Novozym 435, 0.2055 g). A stir bar was added to the vial, which was placed in a constant temperature water bath (T=45° C.) supported by a magnetic stirrer. The reaction was stirred, and monitored periodically by TLC and/or HPLC:

The purity of 20BECD (supplied by FPT, Inc.) was assessed as about −96% by HPLC. A single unidentified peak was observed at slightly longer RT. TLC results corroborated HPLC findings.

The reaction was monitored by HPLC every 24 h for 11 days. At 168 h (7 days), HPLC showed five peaks (RT/Area %): 2.280/58.01 (20BECD); 2.462/1.00 (unknown impurity in 20BECD); 2.600/37.43 (20BECD 2-acetate); 2.899/0.66 (unknown); 3.292/2.83 (unknown).

Reaction 2. This reaction was run in parallel with Reaction 1, and employed the same conditions, except that CH3CN replaced t-amyl alcohol-pyridine.

The reaction was monitored by HPLC every 24 h for 11 days. At 168 h (7 days), HPLC showed five peaks (RT/Area %): 2.280/47.38 (20BECD); 2.464/1.13 (unknown impurity in 20BECD); 2.608/45.09 (20BECD 2-acetate); 2.894/1.64 (unknown); 3.291/4.65 (unknown).

Reaction 3. This reaction was run in an open vial using minimal solvent (DMF, 500 uL), in the presence of molecular sieves, under static conditions at 50° C. After 15 h, HPLC showed >90% unreacted 20BECD, and six additional peaks.

Reaction 4. This reaction was run in a capped, vented vial using a 25-fold molar excess of vinyl acetate and t-amyl alcohol as the sole solvent in the presence of molecular sieves, under static conditions at 50° C.

The reaction was monitored by HPLC every 24 h for 10 days. At 119 H (˜5 days), HPLC showed five peaks (RT/Area %): 2.273/47.05 (20BECD); 2.457/1.78 (unknown impurity in 20BECD); 2.596/46.63 (20 BECD 2-acetate) 2.883/0.62 (unknown); 3.276/3.92 (unknown).

Reaction 5. This reaction was run in a capped vial using a 50-fold molar excess of vinyl acetate and HOAc as the sole solvent in the presence of molecular sieves, under static conditions at 50° C. After 65 h, HPLC showed ˜65% unreacted 20BECD, and four additional peaks at longer RT (7-10%, each).

Reaction 6. This reaction was run in a capped, vented vial using a 25-fold molar excess of vinyl acetate and dioxane as the sole solvent in the presence of molecular sieves, under static conditions at 60° C.

The reaction was monitored by HPLC every 24 h for 7 days. At 42 h, HPLC showed three major peaks (RT/Area %): 2.444/71.52 (20BECD); 2.938/7.51 (unknown impurity in 20BECD); 3.273/19.43 (20BECD 2-acetate). Four additional minor peaks were observed at longer RT (total <2%).

Reaction 7. This reaction was run in a capped vial using a 50-fold molar excess of vinyl acetate and t-amyl alcohol as the sole solvent in the presence of molecular sieves, under static conditions at 60° C.

The reaction was monitored by HPLC at 42 and 114 h. At 42 h, HPLC showed five peaks (RT/Area %): 2.397/45.91 (20BECD); 2.924/7.30 (unknown impurity in 20BECD); 3.271/44.35 (20BECD 2-acetate); 4.535/0.68 (unknown); 4.757/1.77 (unknown).

Reaction 8. This reaction was run as described for Reaction 7, except dioxane replaced t-amyl alcohol.

The reaction was monitored by HPLC at 42 and 114 h. At 42 h, HPLC showed seven peaks (RT/Area %): 2.398/17.10 (20BECD); 2.922/14.48 (unknown impurity in 20BECD); 3.304/62.91 (20BECD 2-acetate). Four additional peaks were observed at longer RT (total ˜5.5%).

Reaction 9. Exhaustive acetylation of 20B-hydroxyecdysone was evaluated according to the procedure of Galbraith and Horn (Aust. J. Chem. 1969, 22, 1045-1057). 20BECD 0.2103 g, 0.438 mmole was weighed into a 20 ml scintillation vial. A stir bar was added followed by pyridine (1000 uL). To the resultant solution was added Ac₂O (1000 uL). The vial was placed in a jacketed beaker maintained at 30° C., and the reaction mixture was stirred for 68 h. Most of the Ac₂O pyridine was removed under a stream of warm air and the residue was triturated with water to give an off-white solid (˜0.28 g). HPLC of the solid showed a single major peak at 11.62 min (>95%). TLC shows two major spots of approximately equal intensity (visualization: 254 nm). Acetylation of 20BECD resulted in nearly quantitative conversion of the starting material to distinctly non-polar compounds (TLC), the tetra- and triacetates.

HPLC Testing Method of 20B-hydroxyecdysone Polyacetates

| | |
|---|---|
| Sample preparation: | dissolve the sample with methanol |
| Testing instrument: | HP1100 |
| Column: | Inertsil ODS-3 5 um |
| | (4.6 × 250 mm) |
| Eluent: | Acetonitrile + 0.1% Phosphinic |
| | acid solution = 60 + 40 (v + v) |
| Detection wavelength: | 254 nm |
| Injection | 20 ul |
| Flow Rate: | 1 ml/min |
| Column Temperature: | 35° C. |
| Retention Times: | |
| 20-B-hydroxy-ecdysone Tetraacetates | 6.0-6.5 mins. |
| 20-B-hydroxy-ecdysone Triacetates | 11.0-11.5 mins. |

A typical HPLC chart is depicted as follows:

[image]

Area Percent Report
Sorted by: Signal
Multiplier: 1.0000
Dilution: 1.0000
Signal 1: VWD1 A, Wavelength = 254 nm
Results obtained with enhanced integrator

| | RetTime | | Width | Area | Height | |
|---|---|---|---|---|---|---|
| Peak # | (min) | Type | (min) | mAU * s | (mAU) | Area % |
| 1 | 5.189 | VV | 0.2380 | 147.38312 | 8.85025 | 0.3056 |
| 2 | 5.589 | VV | 0.1804 | 380.98999 | 31.51444 | 0.7899 |
| 3 | 6.015 | VV | 0.1889 | 2.46825e4 | 2045.00366 | 51.1741 |
| 4 | 6.931 | VV | 0.4041 | 318.91147 | 10.20070 | 0.6612 |
| 5 | 9.036 | VV | 0.2890 | 685.11182 | 33.58520 | 1.4204 |
| 6 | 9.765 | VP | 0.4282 | 188.33344 | 6.00984 | 0.3905 |
| 7 | 11.033 | VP | 0.2711 | 2.04549e4 | 1169.05261 | 42.4090 |
| 8 | 13.537 | VP | 0.2952 | 995.53229 | 52.22947 | 2.0640 |
| 9 | 18.313 | BB | 0.3809 | 378.73016 | 15.43308 | 0.7852 |
| | Totals: | | | 4.82324e4 | 3371.87925 | |

### Administration

In a preferred embodiment, one or more or a mixture of the inventive compounds as nutrients and/or food supplements of the invention may be orally administered to, or by a user, for example, a person desirous of maintaining energy and promoting muscle and other body tissue development, or to combat fatigue, and to reduce muscle damage during exercise, or to enhance recovery after exercise, or for any known efficacy of the amino acid residue present and/or of the tetracyclic compound present or any end result contemplated. For example, a regimen for enhancing physical performance through amino acid nutritional amino acid supplements as disclosed in U.S. Pat. No. 5,026,721 may be used with the inventive compounds of this invention.

In an additional preferred embodiment, for example, the novel compound supplements may be administered orally individually, or in selected mixtures tailored to achieve specific end results, such as various and each of the essential and non-essential amino acids are known for their individual efficacy, or of particular tetracyclic steroidal compounds known for their individual efficacy as used in various indications.

In some preferred embodiments, the compounds of the invention as used as supplements to enhance a mammal's body performance, such as physical performance and the like, may comprise any amount which shows efficacy to any degree, or for example, individually or in combination from about 0.0001% by weight to about 99.99% by weight, or such weight percent ranges of a pharmaceutically acceptable carrier material, and more preferably from about 0.001% by weight to about 20% by weight, or such weight percent ranges of a pharmaceutically acceptable carrier material.

In any event, the inventive compounds are administered in therapeutically effective amounts which are that amount which provides therapeutic affects for a given condition and administration regimen.

Generally, without desiring to limit this invention to any theory, specific hypothesis or biochemical synthetic mode of action, it is thought that the inventive compounds, at least with respect to oral administration, will be hydrolyzed at some point or location in a mammal's body and, especially with respect to amino acid/oligo-peptide esterified derivatives, the individual components of amino acid/oligo-peptide and steroidal-like tetracyclic compound will then be adsorbed or absorbed, thereby providing a double acting compound with double efficacy. The rate of absorption will, of course, vary greatly from person to person. Further, absorption will occur at different rates and in various combinations of different modes of action depending on functionality. For example, the compounds 20-hydroxyecdysone 2,3,22-triacetate and 20-hydroxyecdysone 2-acetate are much less polar than their parent compounds, with the ester functionality making these compounds intrinsically more lipophilic with decreased H-bonding capacity and increased hydrocarbon content. Increased lipophilicity is a property which is oftentimes consistent with enhanced membrane permeability and is often observed to improve absorption after oral administration. More particularly, it has been shown that acyl ecdysteroids are liable to enzymic hydrolysis, and may function as controlled release forms, or pro-drugs, of the ecdysteroid. See, for example, Khimiya Rastitel'nogo Syr'ya, 2001, 2, 69-81 (Russian) (abstract), describing the chemical modification of 20-hydroxyecdysone and the study of membranotropic properties of its derivatives, and finding that ecdysteroid esters are liable to enzymic hydrolysis in vitro.

The compounds of the present invention, or at least their hydrolysis products, and their bio-activity and toxicity are for the most part well known. For example, the amino acid and/or peptide hydrolysis products are well known as to their respective efficacy and toxicity levels. Additionally, phyto-derived tetracyclic steroidal-like compounds, such as phytoecdysteroids and those compounds of the present invention, are found in many plant species and are widely used in many preparations now available on the market. See, for example, Lafont et al., Practical uses for ecdysteroids in mammals including humans: and update, Journal of Insect Science, 3: 7 (March 2003), describing, inter alia, increased physical performance efficacy and other anabolic effects, and also in excess of one-hundred and forty different preparations containing ecdysteroids. Additionally, see Dinan, L., Ecdysteroid structure-activity relationships, Department of Biological Sciences, University of Exter, Studies in Natural Products Chemistry, 29 Bioactive Natural Products, 3-71 (2003); Suksamrarn et al., Synthesis and biological activity of 2-deoxy-20-hydroxyecdysone and derivatives, Tetrahedron, vol. 52, No. 38 pp. 12623-12630 (1996); Synthesis and biological activity of side-chain analogues of ecdysone and 20-hydroxyecdysone, Roussel et al., J. Chem. Soc., Perkin-Trans. 1, 1997; Synthesis and biological activities of turkesterone 11-alpha-acyl derivatives, Dinan et al., Journal of Insect Science, 3: 6 (2002); and Bergamasco et al., The biological activities of ecdysteroids and ecdysteroid analogues, Developments in Endocrinology (Amsterdam), (Prog. Ecdysone. Res) 299-324 (1980).

The phrase “pharmaceutically acceptable carrier” is recognized in the art, and includes a pharmaceutically acceptable material, composition or vehicle, suitable for administering in any way the compound(s) of the present invention to humans or animals. The carriers include, without limitation, liquid or solid filler, diluent, excipient, solvent or encapsulating material, involved in carrying the subject agent and/or compound(s), such as esterified amino acid ester, from one portion of the body, or organ, to another portion of the body, or organ. Each carrier must be “acceptable” in the sense of being compatible with the other ingredients of a formulation, and, of course, not injurious to the patient.

Some examples of materials which can serve as pharmaceutically acceptable carriers include, for instance, sugars, such as lactose, glucose, or sucrose; starches, such as corn starch and potato starch, cellulose and its derivatives, such as carboxymethyl cellulose, ethyl cellulose and cellulose acetate; natural and synthetic water soluble gums, such as powered tragacanth, guar gum and the like; malt; gelatin; talk; excipients, such as cocoa butter and suppository waxes; oils, such as sesame seed oil, peanut oil, palm oil, cottonseed oil, safflower oil, sesame oil, olive oil, corn oil, palm oil and soybean oil; glycols, such as propylene glycol; polyols, such as glycerine, sorbitol, mannitol and polyethylene glycol; natural and synthetic lipid emulsions, such as Interlipid, esters, such as ethyl oleate and ethyllaurate; agar and other gelling and/or thickening agents; pH adjusting and/or buffering agents, such as magnesium hydroxide, aluminum hydroxide and phosphate buffer solutions; alginic acid and salts thereof; saline; isotonic saline; ethyl alcohol; and other non-toxic compatible substances employed in pharmaceutical formulations.

Other agents and compounds which may be employed include, without limitation, wetting agents, emulsifiers and lubricants, such as sodium lauryl sulfate and magnesium stearate; coloring agents, release agents; coating agents; sweetening, flavoring and fragrance agents; preservatives and antioxidants, such as ascorbic acid, cysteine hydrochloride, sodium bisulfate, sodium meta bisulfite, sodium sulfite and the like. Some non-limiting examples of oil-soluble anti-oxidants useful herein include ascorbal palmitate, butylated hydroxyanisole (BHA), butylated hydroxytoluene (BHT), lecithin, propyl gallate and the like. Also useful herein are one or more metal chelating agents, such as citric acid, ethylenediamine tetraacetic acid (EDTA), sorbitol, tartaric acid, phosphoric acid and the like.

While preferred for oral deployment and ingestion, the inventive compounds and formulations thereof of this invention include those suitable for nasal, topical, transdermal, buccal, sublingual, rectal, vaginal, pulmonary and/or parenteral administration, and/or may find use in any of an array of personal consumer body products, such as hair products, hand and body lotions, skin conditioners and in treatments for finger and toenails. Such formulations may be prepared by any conventional method(s) well known in the art of pharmacy, or by any non-conventional method, including convenient preparation by unit dosage form, with some exemplified methods including the step(s) of bring into association one or more compound(s), or amino acid esters, of the invention with one or more carriers and, optionally, one or more accessory ingredients, preferably uniformly and intimately, and if necessary, or desired, shaping the resulting product, and which will be administered by forms suitable for each administration route, such as by tablet and capsule form, by injection, inhalation, eye lotion, lotion or ointment, and suppository.

By “parenteral administration” and “administered parenterally” as used herein, means modes of administration other than enternal and topical administration, and usually by injection, which includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, aubarachnoid, intraspinal and intrastemal injection and infusion.

The terms “systemic administration” and “administered systemically”, “peripheral administration” and “administered peripherally” as used herein mean the administration of one or more of the inventive compound(s), such as amino acid esters, drugs or other material other than directly into the central nervous system, such that it enters a patient's or user's system/body and is subject to metabolism and similar processes, for example, such as subcutaneous administration and oral administration.

Subject to some preferred compounds and their preferred concentration ranges of use as mentioned above, the amount of active ingredient, be it an inventive compound of the invention or other bio-active ingredient, which can be combined with a carrier material to produce a single dosage form will generally be that amount effective to produce a therapeutic effect, or display any amount of efficacy; such as enhanced energy of the user, pain reduction and the like which shall generally referred to herein as a mammal effective nutritional supplement amount. In general, based upon one-hundred percent, this amount will range from less than about 0.0001 percent to more than about 99.999 percent of active ingredient, depending upon such variables such as the nature of the active ingredient employed, the user, the carrier material employed and the end result contemplated. Actual dosage levels of active ingredients in the inventive formulations may be varied so as to obtain an amount of the active ingredient(s) which is effective to achieve the desired end result contemplated or otherwise the desired therapeutic response (effective nutritional supplement amount) for a particular patient or user, composition, and mode of administration.

In another aspect of the invention, there are provided novel methods of conducting one or more business functions comprising the design, production, marketing, distribution, sale, licensing and/or leasing of the inventive compounds, or nutritional supplements formulations thereof and methods of treating and/or administrating same to person's and animals in need or desire thereof. The novel compounds, formulations and methods of the invention provide unique business opportunities heretofore unavailable, and which will enable the capture of a distinct and exclusive market share for its owners and licensees in the important health services sector with the invention's advantages over conventional products and methodology as described. It is further contemplated that the inventive subject matter herein be employed as a valuable business tool in the generation of business goodwill and as a vehicle for use in conjunction with trade-marks to generate valuable source identifiers, and as subject matter to form and operate a business entity.

It is to be understood that many modifications and variations besides those preferred embodiments described above may be made in the inventive nutritional compositions of the invention without departing from the scope and spirit of invention and claims. It is also to be understood that the above embodiments are for illustrative purposes only and are not intended to limit the invention and for appended claims in any way.

1. A nutritional supplement and/or pharmaceutical composition comprising a compound selected from the formulae: 1 through 383, and
wherein R is one or more of a biologically significant amino acid residue, or a residue of a naturally occurring amino acid, a peptide residue, an oligo-peptide residue and pharmaceutically acceptable salts thereof,
or R is one or more of hydrogen, hydroxy, halogen, alkyl, cycloalkyl, alkyl, substituted alkyl, cycloalkyl, substituted cycloalkyl, heterocylclic, substituted heterocyclic, alkenyl, substituted alkenyl, alkynl, substituted alkynyl, aryl, substituted aryl, heteroaryl, substituted hereroaryl, alkylaryl, substituted alkylaryl, arylalkyl, substituted arylalkenyl, substituted arylalkenyl, arylalkynl, substituted arylalkynl, aroyl, substituted aroyl, acyl, substituted acyl, or the like, or the two R groups can cooperate to form a 5-, 6- or 7-membered ring including N and the two R groups, or either of the R groups is a divalent moiety selected from the group consisting of alkylene, substituted alkylene, oxyalklene, substituted oxyalkylene, alkenylene, substituted alkenylene, arylene, substituted arylene, alkarylene, substituted alkarylene, aralkylene and substituted aralkylene, wherein said divalent moiety serves as the same substituent for two dithiocarbamate structures, thereby linking said structures together so as to form a bis(dithiocarbamate) species, and pharmaceutical acceptable salts thereof.

2. The nutritional supplement of claim 1 wherein R is a residue of essential, non-essential and biologically significant amino acids, peptides thereof, oligo-peptides thereof and/or pharmaceutically acceptable salts thereof.

3. The nutritional supplement of claim 2 wherein R is selected from the group consisting of glycine, alanine, valine, leucine, isoleucine, phenylalanine, proline, serine, threonine, tyrosine, cysteine, methionine, lysine, arginine, histidine, tryptophan, aspartic acid, glutamic acid, asparagine, glutamine, taurine, and citrulline or peptides or oligo-peptides composed thereof, or pharmaceutically acceptable salts thereof.

4. The nutritional supplement of claim 1 wherein said compound is present in amounts ranging from about 0.0001 weight % to about 99.999 weight % of the nutritional supplement.

5. The nutritional supplement of claim 1 which may be administered to mammals by way of one or more selected from the group consisting of oral, nasal, topical, transdermal, buccal, sublingual, rectal, vaginal, pulmonary and parenteral administration.

6. The nutritional supplement of claim 1 wherein said supplement further comprises a pharmaceutically acceptable carrier.

7. The nutritional supplement of claim 6 wherein said compound is present as a pharmaceutically acceptable salt.

8. The nutritional supplement of claim 6 further comprising one or more of additional bio-active compositions, vitamins, minerals, electrolytes and carbohydrates, excipients and adjuvants.

9. A method for the production of the nutritional supplement of claim 1.

10. A method for the production of the nutritional supplement of claim 2.

11. A method for the production of the nutritional supplement of claim 3.

12. A method for the production of the nutritional supplement of claim 4.

13. A method for the production of the nutritional supplement of claim 5.

14. A method for the production of the nutritional supplement of claim 6.

15. A method for the production of the nutritional supplement of claim 7.

16. A method for the production of the nutritional supplement of claim 8.

17. A method for treating a person or animal in need thereof comprising the administration thereof of a nutritional supplement of claim 1.

18. A method for treating a person or animal in need thereof comprising the administration thereof of a nutritional supplement of claim 2.

19. A method for treating a person or animal in need thereof comprising the administration thereof of a nutritional supplement of claim 3.

20. A method for treating a person or animal in need thereof comprising the administration thereof of a nutritional supplement of claim 4.

21. A method for treating a person or animal in need thereof comprising the administration thereof of a nutritional supplement of claim 5.

22. A method for treating a person or animal in need thereof comprising the administration thereof of a nutritional supplement of claim 6.

23. A method for treating a person or animal in need thereof comprising the administration thereof of a nutritional supplement of claim 7.

24. A method for treating a person or animal in need thereof comprising the administration thereof of a nutritional supplement of claim 8.

25. A method of conducting one or more business functions selected from designing, manufacturing, using, marketing, distributing, selling, licensing, and leasing comprising the use of the nutritional supplement of claim 1.

26. A method of conducting one or more business functions selected from designing, manufacturing, using, marketing, distributing, selling, licensing, and leasing comprising the use of the nutritional supplement of claim 2.

27. A method of conducting one or more business functions selected from designing, manufacturing, using, marketing, distributing, selling, licensing, and leasing comprising the use of the nutritional supplement of claim 3.

28. A method of conducting one or more business functions selected from designing, manufacturing, using, marketing, distributing, selling, licensing, and leasing comprising the use of the nutritional supplement of claim 4.

29. A method of conducting one or more business functions selected from designing, manufacturing, using, marketing, distributing, selling, licensing, and leasing comprising the use of the nutritional supplement of claim 5.

30. A method of conducting one or more business functions selected from designing, manufacturing, using, marketing, distributing, selling, licensing, and leasing comprising the use of the nutritional supplement of claim 6.

31. A method of conducting one or more business functions selected from designing, manufacturing, using, marketing, distributing, selling, licensing, and leasing comprising the use of the nutritional supplement of claim 7.

32. A method of conducting one or more business functions selected from designing, manufacturing, using, marketing, distributing, selling, licensing, and leasing comprising the use of the nutritional supplement of claim 8.
